(19) United States
(12) Patent Application Publication
(10) Pub. No.: US 2025/0002575 A1
(43) Pub. Date: Jan. 2, 2025

(54) ANTI-INTERLEUKIN-33 ANTIBODIES AND USES THEREOF

(71) Applicant: Genentech, Inc., South San Francisco, CA (US)

(72) Inventors: John Hok Nin LOWE, Dublin, CA (US); Kelly Michele LOYET, San Mateo, CA (US); Elaine MAI, San Francisco, CA (US); Mauricio Silva MAIA, Belmont, CA (US); Gerald R. NAKAMURA, San Francisco, CA (US); Jack BEVERS, III, San Francisco, CA (US); Hiu Nam CHAN, Union City, CA (US); Laetitia D. COMPS-AGRAR, Mill Valley, CA (US); Racquel CORPUZ, South San Francisco, CA (US)

(73) Assignee: Genentech, Inc., South San Francisco, CA (US)

(21) Appl. No.: 18/658,118

(22) Filed: May 8, 2024

Related U.S. Application Data
(63) Continuation of application No. PCT/US2022/079523, filed on Nov. 9, 2022.
(60) Provisional application No. 63/277,953, filed on Nov. 10, 2021.
(60) Provisional application No. 63/413,206, filed on Oct. 4, 2022.

(51) Int. Cl. C07K 16/24; G01N 33/68

(52) U.S. Cl. CPC C07K 16/244; G01N 33/6869; G01N 33/6878; C07K 2317/92; G01N 2333/54

(57) ABSTRACT

The invention provides interleukin-33 (IL-33) antibodies and methods of making and using the same, e.g., for detection of IL-33 in biological samples.

### CROSS-REFERENCE TO RELATED APPLICATIONS

The present application is a continuation of International Patent Application No. PCT/US2022/079523 filed Nov. 9, 2022 which claims the benefit of priority of U.S. Provisional Application No. 63/277,953, filed Nov. 10, 2021, and U.S. Provisional Application No. 63/413,206 filed Oct. 4, 2022, the contents of each of which are incorporated herein by reference in their entirety.

### SEQUENCE LISTING

The instant application contains a Sequence Listing which has been submitted electronically in XML format and is hereby incorporated by reference in its entirety. Said XML copy, created on May 5, 2024, is named P37118-US-2_Seq_List.XML and is 38,312 bytes in size.

### FIELD OF THE INVENTION

The invention relates to anti-interleukin-33 (IL-33) antibodies, and methods of making and using the same, including detection of IL-33 in biological samples.

### BACKGROUND

Interleukin-33 (IL-33) is a member of the interleukin-1 (IL-1) cytokine family that is encoded by the IL33 gene, and is expressed in structural cells, such as smooth muscle, epithelial (e.g., retinal pigment epithelium (RPE) cells), endothelial cells (e.g., choroidal endothelial cells), Müller cells, and astrocytes. IL-33 can be induced by inflammatory factors in macrophages and dendritic cells. Over the past decade, human IL-33 (hIL-33) has emerged as a key contributor to the pathogenesis of numerous inflammatory diseases. Cellular stress caused by environmental triggers, such as allergens, toxins, and pathogens, can lead to IL-33 release. For example, IL-33 is released from Müller cells upon phototoxic stress and induces CCL₂ and monocyte-dependent retinal degeneration in preclinical models. Bioavailable IL-33 associates with a heterodimeric IL-33 receptor complex composed of suppression of tumorigenicity 2 (ST2) protein and interleukin-1 receptor accessory protein (IL-1RAcP) to activate the AP-1 and NF-κB pathways through the adaptor protein myeloid differentiation primary response 88 (MyD88) and possibly MyD88-adapter-like (Mal) protein. IL-33 stimulates a number of cell types, including innate type II (ILC2) cells, mast cells, basophils, eosinophils, and dendritic cells, to promote Type 2 immunity.

Despite the existence of several commercial hIL-33 assays spanning multiple platform technologies, the ability to provide accurate hIL-33 concentration measurements and to differentiate between active (reduced) and inactive (oxidized) hIL-33 in various matrices remains uncertain. This is especially true for lower sample volumes, matrices with low hIL-33 concentrations, and matrices with elevated levels of soluble Interleukin 1 Receptor-Like 1 (sST2), an inactive form of ST2 that competes with membrane bound ST2 for hIL-33 binding. The accurate measurement of active human IL-33 (hIL-33) in biological matrices remains a challenge for at least the following reasons: 1) in non-pathological to moderate pathological conditions, hIL-33 is often present at undetectable to low levels (<pg/mL), 2) hIL-33 exists in different structural forms (reduced versus oxidized), and 3) hIL-33 can be unbound or bound to human sST2 (hsST2), which can interfere with its detection.

Accurate and sensitive assays for detecting all forms of IL-33, whether unbound or bound to hsST2, are needed.

### SUMMARY

The present invention relates, inter alia, to anti-IL-33 antibodies and methods of making and using the same. The present disclosure provides antibodies with high sensitivity (e.g., binding affinity) to all forms of IL-33, both unbound and associated, as well as methods, assays, kits that comprise and use said antibodies.

In one aspect, the invention features an isolated antibody that specifically binds IL-33, or an antigen-binding fragment thereof, wherein the antibody comprises a binding domain comprising a heavy chain variable (VH) domain and a light chain variable (VL) domain, the binding domain comprising the following six complementarity-determining regions (CDRs): (a) a CDR-H1 comprising the amino acid sequence of SEQ ID NO: 1; (b) a CDR-H2 comprising the amino acid sequence of SEQ ID NO: 2; (c) a CDR-H3 comprising the amino acid sequence of SEQ ID NO: 3; (d) a CDR-L1 comprising the amino acid sequence of SEQ ID NO: 4; (e) a CDR-L2 comprising the amino acid sequence of SEQ ID NO: 5; and (f) a CDR-L3 comprising the amino acid sequence of SEQ ID NO: 6.

In some aspects, the VH domain comprises the following framework regions (FRs): (a) an FR-H1 comprising the amino acid sequence of SEQ ID NO: 11; (b) an FR-H2 comprising the amino acid sequence of SEQ ID NO: 12; (c) an FR-H3 comprising the amino acid sequence of SEQ ID NO: 13; and (d) an FR-H4 comprising the amino acid sequence of SEQ ID NO: 14.

In some aspects, the VL domain comprises the following FRs: (a) an FR-L1 comprising the amino acid sequence of SEQ ID NO: 15; (b) an FR-L2 comprising the amino acid sequence of SEQ ID NO: 16; (c) an FR-L3 comprising the amino acid sequence of SEQ ID NO: 17; and (d) an FR-L4 comprising the amino acid sequence of SEQ ID NO: 18.

In some aspects, the antibody disclosed herein comprises (a) a VH domain comprising an amino acid sequence having at least 95% (e.g., 96%, 97%, 98%, 99%, or 100%) sequence identity to the amino acid sequence of SEQ ID NO: 7; (b) a VL domain comprising an amino acid sequence having at least 95% (e.g., 96%, 97%, 98%, 99%, or 100%) sequence identity to the amino acid sequence of SEQ ID NO: 8; or (c) a VH domain as in (a) and a VL domain as in (b).

In some aspects, the VH domain comprises the amino acid sequence of SEQ ID NO: 7.

In some aspects, the VL domain comprises the amino acid sequence of SEQ ID NO: 8.

In one aspect, the invention provides an isolated antibody that specifically binds IL-33, or an antigen-binding fragment thereof, wherein the antibody comprises a binding domain comprising (a) a VH domain comprising the amino acid sequence of SEQ ID NO: 7 and (b) a VL domain comprising the amino acid sequence of SEQ ID NO: 8.

In some aspects, the antibody disclosed herein comprises a heavy chain (HC) having at least 95% (e.g., 96%, 97%, 98%, 99%, or 100%) sequence identity to the amino acid sequence of SEQ ID NO: 9; (b) a light chain (LC) having at least 95% (e.g., 96%, 97%, 98%, 99%, or 100%) sequence identity to the amino acid sequence of SEQ ID NO: 10; or (c) an HC as in (a) and an LC as in (b).

In some aspects, the HC comprises the amino acid sequence of SEQ ID NO: 9.

In some aspects, the LC comprises the amino acid sequence of SEQ ID NO: 10.

In one aspect, the invention provides an isolated antibody that specifically binds IL-33, or an antigen-binding fragment thereof, wherein the antibody comprises an HC comprising the amino acid sequence of SEQ ID NO: 9 and an LC comprising the amino acid sequence of SEQ ID NO: 10.

In one aspect, the invention provides an isolated antibody that specifically binds IL-33, or an antigen-binding fragment thereof, wherein the antibody comprises a binding domain comprising the following six CDRs: (a) a CDR-H1 comprising the amino acid sequence of SEQ ID NO: 19; (b) a CDR-H2 comprising the amino acid sequence of SEQ ID NO: 20; (c) a CDR-H3 comprising the amino acid sequence of SEQ ID NO: 21; (d) a CDR-L1 comprising the amino acid sequence of SEQ ID NO: 22; (e) a CDR-L2 comprising the amino acid sequence of SEQ ID NO: 23; and (f) a CDR-L3 comprising the amino acid sequence of SEQ ID NO: 24.

In some aspects, the VH domain comprises the following FRs: (a) an FR-H1 comprising the amino acid sequence of SEQ ID NO: 29; (b) an FR-H2 comprising the amino acid sequence of SEQ ID NO: 30; (c) an FR-H3 comprising the amino acid sequence of SEQ ID NO: 31; and (d) an FR-H4 comprising the amino acid sequence of SEQ ID NO: 32.

In some aspects, the VL domain comprises the following FRs: (a) an FR-L1 comprising the amino acid sequence of SEQ ID NO: 33; (b) an FR-L2 comprising the amino acid sequence of SEQ ID NO: 34; (c) an FR-L3 comprising the amino acid sequence of SEQ ID NO: 35; and (d) an FR-L4 comprising the amino acid sequence of SEQ ID NO: 36.

In some aspects, the antibody disclosed herein comprises (a) a VH domain having at least 95% (e.g., 96%, 97%, 98%, 99%, or 100%) sequence identity to the amino acid sequence of SEQ ID NO: 25; (b) a VL domain having at least 95% (e.g., 96%, 97%, 98%, 99%, or 100%) sequence identity to the amino acid sequence of SEQ ID NO: 26; or (c) a VH domain as in (a) and a VL domain as in (b).

In some aspects, the VH domain comprises the amino acid sequence of SEQ ID NO: 25.

In some aspects, the VL domain comprises the amino acid sequence of SEQ ID NO: 26.

In one aspect, the invention provides an isolated antibody that specifically binds IL-33, or an antigen-binding fragment thereof, wherein the antibody comprises a binding domain comprising (a) a VH domain comprising the amino acid sequence of SEQ ID NO: 25 and (b) a VL domain comprising the amino acid sequence of SEQ ID NO: 26.

In some aspects, the antibody comprises an HC having at least 95% (e.g., 96%, 97%, 98%, 99%, or 100%) sequence identity to the amino acid sequence of SEQ ID NO: 27; (b) an LC having at least 95% (e.g., 96%, 97%, 98%, 99%, or 100%) sequence identity to the amino acid sequence of SEQ ID NO: 28; or (c) an HC as in (a) and an LC as in (b).

In some aspects, the HC comprises the amino acid sequence of SEQ ID NO: 27.

In some aspects, the LC comprises the amino acid sequence of SEQ ID NO: 28.

In one aspect, the invention provides an isolated antibody that specifically binds IL-33, or an antigen-binding fragment thereof, wherein the antibody comprises an HC comprising the amino acid sequence of SEQ ID NO: 27 and an LC comprising the amino acid sequence of SEQ ID NO: 28.

In some aspects, the antibody binds to oxidized or reduced IL-33.

In some aspects, the antibody binds to oxidized IL-33 and to reduced IL-33 with within two-fold (e.g., within one-and-a-half-fold or within one-fold) K_{D}. In some aspects, the antibody binds to oxidized IL-33 and to reduced IL-33 with substantially the same K_{D}.

In some aspects, the antibody binds to reduced IL-33. In some aspects, the antibody binds to reduced IL-33 with a K_{D}≤1 nM. In some aspects, the antibody binds to reduced IL-33 with a K_{D} between 0.01 nM and 1 nM (e.g., between 0.01 and 0.9 nM, between 0.01 and 0.8 nM, between 0.01 and 0.7 nM, between 0.01 and 0.6 nM, between 0.01 and 0.5 nM, between 0.05 and 0.9 nM, between 0.1 and 0.9 nM, between 0.2 and 0.9 nM, between 0.3 and 0.9 nM, between 0.2 and 0.6 nM, between 0.3 and 0.5 nM, between 0.3 and 0.4 nM, or between 0.4 and 0.5 nM; e.g., about 0.05 nM, about 0.1 nM, about 0.2 nM, about 0.3 nM, about 0.325 nM, about 0.34 nM, about 0.35 nM, about 0.4 nM, about 0.45 nM, about 0.475 nM, about 0.49 nM, about 0.5 nM, about 0.6 nM, about 0.7 nM, about 0.8 nM, about 0.9 nM, or about 1 nM). In some aspects, the antibody binds to reduced IL-33 with a K_{D}≤0.5 nM. In some aspects, the antibody binds to reduced IL-33 with a K_{D} between 0.1 nM and 0.5 nM (e.g., between 0.2 and 0.5 nM, between 0.3 nM and 0.5 nM, between 0.3 nM and 0.4 nM, or between 0.4 nM and 0.5 nM). In some aspects, the antibody binds to reduced IL-33 with a K_{D} of about 0.487 nM. In some aspects, the antibody binds to reduced IL-33 with a K_{D} of about 0.338 nM.

In some aspects, the antibody binds to human IL-33.

In some aspects, the antibody binds to IL-33 bound to soluble interleukin 1 receptor like 1 (sST2) protein and to IL-33 not bound to sST2 with within two-fold (e.g., within one-and-a-half-fold or within one-fold) K_{D}. In some aspects, the antibody binds to IL-33 bound to sST2 protein with substantially the same K_{D} as to IL-33 not bound to sST2. In some aspects, the sST2 is human sST2 (hsST2).

In some aspects, the antibody is a monoclonal antibody.

In some aspects, the antibody is a chimeric antibody.

In some aspects, the antibody is a rat antibody.

In some aspects, the antibody is an IgG antibody.

In some aspects, the antibody is an IgG1 antibody. In some aspects, the antibody is a human IgG1 antibody.

In some aspects, the antibody is an IgG2a antibody. In some aspects, the antibody is a rat IgG2a antibody.

In some aspects, the antibody is an antibody fragment that specifically binds IL-33.

In some aspects, the antibody fragment is selected from the group consisting of Fab, single chain variable fragment (scFv), Fv, Fab′, Fab′-SH, F (ab′)₂, and diabody.

In one aspect, the invention provides an isolated nucleic acid encoding any one of antibodies disclosed herein or a set of isolated nucleic acids together encoding any one of antibodies disclosed herein.

In one aspect, the invention provides a vector or a set of vectors comprising any one of isolated nucleic acids or any one of sets of isolated nucleic acids described herein.

In one aspect, the invention provides a host cell comprising any one of vectors or any one of sets of vectors described herein. In some aspects, the host cell is a mammalian cell. In some aspects, the mammalian cell is a rat cell. In some aspects, the mammalian cell is a Chinese hamster ovary (CHO) cell. In some aspects, the host cell is a prokaryotic cell. In some aspects, the prokaryotic cell is E. coli.

In one aspect, the invention provides a method of producing an antibody that specifically binds to IL-33, the method comprising culturing any one of host cells described herein in a culture medium. In some aspects, the method further comprises recovering the antibody from the host cell or the culture medium.

In one aspect, the invention provides an immunoconjugate comprising any one of antibodies described herein.

In one aspect, the invention provides any one of the antibodies or immunoconjugates described herein for use in detecting the presence or level of IL-33 in a biological sample. In one aspect, the invention provides any one of the antibodies described herein for use in detecting the presence or level of IL-33 in a biological sample. In one aspect, the invention provides any one of the immunoconjugates described herein for use in detecting the presence or level of IL-33 in a biological sample.

In some aspects, the detecting is by immunohistochemistry (IHC), immunofluorescence (IF), flow cytometry, Enzyme-Linked Immunosorbant Assay (ELISA), immunoblotting, or immune-polymerase chain reaction (iPCR). In some aspects, the detecting is by ELISA. In some aspects, the detecting is by iPCR.

In some aspects, the biological sample is a blood sample, an ocular sample, a nasal sample, or a cell culture sample. In some aspects, the blood sample is a plasma or serum sample. In some aspects, the ocular sample is a vitreous humor or aqueous humor sample. In some aspects, the ocular sample is a tissue sample. In some aspects, the nasal sample is a nasosorption fluid sample.

In some aspects, biological sample is from a subject having, or at risk of, an IL-33-mediated disorder. In some aspects, the IL-33-mediated disorder is an ocular disorder, an inflammatory condition, an immune disorder, a fibrotic disorder, an eosinophilic disorder, an infection, pain, a central nervous system disorder, or a solid tumor.

In some aspects, the ocular disorder is age-related macular degeneration (AMD), retinopathy of the eye, polypoidal choroidal vasculopathy (PCV), diabetic macular edema, dry eye disease, Behcet's disease, retina detachment, glaucoma, uveitis, retinitis pigmentosa, Leber Congenital Amaurosis, Stargardt's disease, traumatic eye injury, or conjunctivitis. In some aspects, the AMD is geographic atrophy (GA), wet AMD, or dry AMD. In some aspects, the AMD is GA. In some aspects, the AMD is intermediate AMD or advanced AMD. In some aspects, the retinopathy of the eye is diabetic retinopathy (DR) or retinopathy of prematurity (ROP). In some aspects, the retinopathy of the eye is high-altitude DR. In some aspects, the conjunctivitis is infectious conjunctivitis or non-infectious conjunctivitis. In some aspects, the conjunctivitis is allergic conjunctivitis.

In some aspects, the inflammatory condition is asthma, sepsis, septic shock, atopic dermatitis, allergic rhinitis, rheumatoid arthritis, or chronic obstructive pulmonary disease (COPD).

In some aspects, the immune disorder is asthma, rheumatoid arthritis, allergy, anaphylaxis, anaphylactic shock, allergic rhinitis, psoriasis, inflammatory bowel disease (IBD), Crohn's disease, diabetes, or liver disease.

In some aspects, the fibrotic disease is idiopathic pulmonary fibrosis (IPF).

In some aspects, the eosinophilic disorder is an eosinophil-associated gastrointestinal disorder (EGID). In some aspects, the EGID is eosinophilic esophagitis.

In some aspects, the subject is human.

In some aspects, the presence or level of IL-33 is the presence or level of total IL-33. In some aspects, the presence or level of IL-33 is the presence or level of reduced IL-33. In some aspects, the presence or level of IL-33 is the presence or level of human IL-33. In some aspects, the presence or level of IL-33 is the presence or level of unbound IL-33. In some aspects, the presence or level of IL-33 is the presence or level of IL-33 bound to sST2. In some aspects, the sST2 is hsST2.

In one aspect, the invention provides a method of detecting the presence or level of IL-33 in a biological sample comprising contacting the biological sample with any one of antibodies or immunoconjugates described herein and detecting the presence of the bound antibody or immunoconjugate. In one aspect, the invention provides a method of detecting the presence or level of IL-33 in a biological sample comprising contacting the biological sample with any one of the antibodies described herein and detecting the presence of the bound antibody. In one aspect, the invention provides a method of detecting the presence or level of IL-33 in a biological sample comprising contacting the biological sample with any one of the immunoconjugates described herein and detecting the presence of the immunoconjugate.

In one aspect, the invention provides a method of detecting the presence or level of IL-33 in a biological sample comprising contacting the biological sample with any one of the immunoconjugates described herein in the presence of a second anti-IL-33 antibody that specifically binds an epitope on IL-33 that is different from the epitope to which the anti-IL-33 antibody in the immunoconjugate specifically binds, and detecting the presence of the bound immunoconjugate, and detecting the presence of the bound immunoconjugate. In one aspect, the second anti-IL-33 antibody is 1E1v8. In one aspect, the biological sample is treated with tris (2-carboxyethyl) phosphine (TCEP) prior to said contacting.

In some aspects, the detecting is by IHC, IF, flow cytometry, ELISA, immunoblotting, or iPCR. In some aspects, the detecting is by ELISA. In some aspects, the detecting is by iPCR.

In some aspects, the biological sample is a blood sample, an ocular sample, a nasal sample, or a cell culture sample. In some aspects, the blood sample is a plasma or serum sample. In some aspects, the ocular sample is a vitreous humor or aqueous humor sample. In some aspects, the ocular sample is a tissue sample. In some aspects, the nasal sample is a nasosorption fluid (NF) sample.

In some aspects, the biological sample is from a subject having, or at risk of, an IL-33-mediated disorder. In some aspects, the IL-33-mediated disorder is an ocular disorder, an inflammatory condition, an immune disorder, a fibrotic disorder, an eosinophilic disorder, an infection, pain, a central nervous system disorder, or a solid tumor.

In some aspects, the ocular disorder is AMD, retinopathy of the eye, PCV, diabetic macular edema, dry eye disease, Behcet's disease, retina detachment, glaucoma, uveitis, retinitis pigmentosa, Leber Congenital Amaurosis, Stargardt's disease, traumatic eye injury, or conjunctivitis. In some aspects, the AMD is GA, wet AMD, or dry AMD. In some aspects, the AMD is GA. In some aspects, the AMD is intermediate AMD or advanced AMD. In some aspects, the retinopathy of the eye is DR or ROP. In some aspects, the retinopathy of the eye is high-altitude DR. In some aspects, the conjunctivitis is infectious conjunctivitis or non-infectious conjunctivitis. In some aspects, the conjunctivitis is allergic conjunctivitis.

In some aspects, the inflammatory condition is asthma, sepsis, septic shock, atopic dermatitis, allergic rhinitis, rheumatoid arthritis, or COPD.

In some aspects, the immune disorder is asthma, rheumatoid arthritis, allergy, anaphylaxis, anaphylactic shock, allergic rhinitis, psoriasis, IBD, Crohn's disease, diabetes, or liver disease.

In some aspects, the fibrotic disease is IPF.

In some aspects, the eosinophilic disorder is an EGID. In some aspects, the EGID is eosinophilic esophagitis.

In some aspects, the subject is human.

In some aspects, the presence or level of IL-33 is the presence or level of total IL-33. In some aspects, the presence or level of IL-33 is the presence or level of reduced IL-33. In some aspects, the presence or level of IL-33 is the presence or level of human IL-33. In some aspects, the presence or level of IL-33 is the presence or level of unbound IL-33. In some aspects, the presence or level of IL-33 is the presence or level of IL-33 bound to sST2. In some aspects, the sST2 is hsST2.

In some aspects, the method further comprises selecting a treatment comprising an IL-33 axis binding antagonist for the subject based on the presence or level of IL-33 in the biological sample.

In some aspects, the method further comprises administering a therapeutically effective amount of an IL-33 axis binding antagonist to the subject.

In some aspects, the IL-33 axis binding antagonist is an IL-33 binding antagonist. In some aspects, the IL-33 binding antagonist is an anti-IL-33 antibody.

In one aspect, the invention provides an assay for identifying a subject having an IL-33 mediated disorder who is a candidate for treatment comprising an IL-33 axis binding antagonist, wherein the assay comprises determining the presence or level of IL-33 in a biological sample obtained from the subject using any one of the antibodies or immunoconjugates described herein. In one aspect, the invention provides an assay for identifying a subject having an IL-33 mediated disorder who is a candidate for treatment comprising an IL-33 axis binding antagonist, wherein the assay comprises determining the presence or level of IL-33 in a biological sample obtained from the subject using any one of the antibodies described herein. In one aspect, the invention provides an assay for identifying a subject having an IL-33 mediated disorder who is a candidate for treatment comprising an IL-33 axis binding antagonist, wherein the assay comprises determining the presence or level of IL-33 in a biological sample obtained from the subject using any one of the immunoconjugates described herein.

In one aspect, the invention provides a kit comprising: (a) any one of the antibodies described herein; and (b) a package insert comprising instructions for use of the antibody to detect the presence or level of IL-33 in a biological sample.

In one aspect, the invention provides a kit comprising: (a) any one of the immunoconjugates described herein; and (b) a package insert comprising instructions for use of the immunoconjugate to detect the presence or level of IL-33 in a biological sample.

### BRIEF DESCRIPTION OF THE DRAWINGS

FIG. 1 is a plot showing hsST2 concentrations determined in vitreous humor (n=22 samples) and serum (n=53 samples) from control donors.

FIG. 2A is an image of an SDS-PAGE gel analysis of hIL-33 diluted in either phosphate-buffered saline (PBS) (a) or 60% Iscove's Modified Dulbecco's Medium (IMDM) (b) and incubated at 37° C. Shown are samples taken at t=0 and t=18 hour time intervals. The protein samples were run under non-reducing conditions and reducing conditions with dithiothreitol (DTT). Molecular weight standards were applied in lanes 3, 6 and 9.

FIG. 2B is a set of mass spectrograms showing mass spectrometry analysis of hIL-33 incubated at 37° C. in (a) PBS at t=0, (b) PBS at t=18 h and (c) IMDM media at t=18 hour. The detected molecular weights (MWs) for hIL-33 in each spectrum are shown. The theoretical MW of hIL-33 with all of its 4 cysteine residues fully reduced is 19857.19 Dalton (Da). For a fully oxidized hIL-33, the theoretical molecular weight (MW) with all 4 cysteines forming two intra-disulfide bonds is 19853.16 Da. Panels (a) and (b) show hIL-33 with its cysteine residues in a fully reduced state while panel (c) shows the hIL-33's cysteine residues in an oxidized state.

FIG. 3 is a plot showing the comparison of hIL-33 standard curves established using eBioscience hIL-33 platinum ELISA kit and hIL-33 provided with the kit (circle), in-house reduced hIL-33 (triangle), and oxidized hIL-33 (inverted triangle). Data are means±SD of triplicates.

FIG. 4A is a plot showing the comparison of standard curves obtained with reduced hIL-33, oxidized hIL-33, reduced hIL-33+20 ng/mL hsST2, reduced hIL-33+40 ng/mL hsST2. Bottom line corresponds to standard curve obtained with oxidized hIL-33.

FIG. 4B is a plot showing the comparison of reduced and oxidized hIL-33 standard curves in presence of 20 ng/mL hsST2. Data are representative of three experiments performed in duplicates.

FIG. 4C is a plot showing the specificity of total hIL-33 ELISA determined by immunodepletion of reduced and oxidized hIL-33 in diluted human vitreous humor samples using beads conjugated to hIL-33 Ab. P=0.0019. Data are means±SD of duplicates.

FIG. 5A is a plot showing the multi-cycle kinetics sensorgrams for hIL-33 binding to captured rat anti-hIL-33 15.21C7 MAb at 25° C. as determined by BIACORE® surface plasmon resonance.

FIG. 5B is a plot showing the multi-cycle kinetics sensorgrams for hIL-33 binding to captured rat/human chimeric anti-hIL-33 3F10 MAb at 25° C. as determined by BIACORE® surface plasmon resonance.

FIG. 6A is a plot showing the reduced hIL-33 standard curve obtained using iPCR in sample buffer. Data are means±SD of 6 independent standard curves, each performed with 4 replicates.

FIG. 6B is a plot showing the specificity of the iPCR assay against reduced hIL-33 as determined by immunodepletion of reduced hIL-33 in diluted human vitreous humor samples using beads conjugated to rat anti-reduced hIL-33 15.21C7 IgG2a, control beads conjugated to rat anti-gp120 IgG2a and sample buffer. P=0.0004 (1:2 vitreous humor), P=<0.0001 (1:4 vitreous humor). Data are mea±SD of triplicates.

FIG. 7A is a plot showing the concentrations of reduced hIL-33 in postmortem human vitreous humor samples from seventeen control and nineteen AMD donors. P=0.0155. Data are means of duplicates. LLOQ=lower limit of quantitation.

FIG. 7B is a plot showing the concentrations of reduced hIL-33 in postmortem human AH. samples from ten control and nine AMD donors. ns=no significance. Data are means of duplicates. LLOQ=lower limit of quantitation.

FIG. 8A is a plot showing the concentrations of reduced hIL-33 in human nasosorption fluid (NF) from nine control and nine asthma patients. ns=no significance. Data are means of duplicates or quadruplicates. LLOQ=lower limit of quantitation.

FIG. 8B is a plot showing the concentrations of reduced hIL-33 in human serum from nine control and nine asthma patients. ns=no significance. Data are means of duplicates or quadruplicates. LLOQ=lower limit of quantitation.

FIG. 8C is a plot showing the concentrations of reduced hIL-33 in human plasma from nine control and nine asthma patients. ns=no significance. Data are means of duplicates or quadruplicates. LLOQ=lower limit of quantitation.

FIG. 8D is a plot showing the correlation of total versus reduced hIL-33 concentrations in serum (circles) and NF (triangles) samples from asthmatic donors (n=23). R²=0.95, P<0.0001 for NF. Data are means of duplicates or quadruplicates. LLOQ=lower limit of quantitation.

FIG. 9A is a plot showing the concentrations of total hIL-33 in human nasosorption fluid (NF) from nine control and nine asthma patients. ns=no significance. Data are means of duplicates or quadruplicates. LLOQ=lower limit of quantitation.

FIG. 9B is a plot showing the concentrations of total hIL-33 in human serum from nine control and nine asthma patients. ns=no significance. Data are means of duplicates or quadruplicates. LLOQ=lower limit of quantitation.

FIG. 9C is a plot showing the concentrations of total hIL-33 in human plasma from nine control and nine asthma patients. ns=no significance. Data are means of duplicates or quadruplicates. LLOQ=lower limit of quantitation.

FIG. 10A is a plot showing the concentrations of hsST2 in human serum from nine control and nine asthma patients. ns=no significance. Data are means of quadruplicates.

FIG. 10B is a plot showing the concentrations of total hIL-33 in human nasosorption fluid (NF) from nine control and nine asthma patients. ns=no significance. Data are means of duplicates or quadruplicates.

FIG. 11 is a schematic diagram showing the steps of an exemplary IL-33 iPCR assay. MM=master mix; SEC=size exclusion chromatography.

FIG. 12 is a plot showing the detectability of reduced IL-33 in various humor, serum, and plasma samples with normal and disease states using a reduced IL-33 iPCR assay. AMD=Age-related macular degeneration; AH=aqueous humor; HS=human serum; Hu=human; N=total number of sample; NH=normal human; NHP=normal human plasma; NHS=normal human serum; NZ=New Zealand; RA=rheumatoid arthritis; %=number of samples>lower limit of quantitation (LLOQ)/N×100.

FIG. 13 is a plot showing the detectability of reduced and total IL-33 in various human humor, serum, and plasma samples with normal and disease states using reduced or total IL-33 iPCR assays. AMD=Age-related macular degeneration; AH=aqueous humor; HS=human serum; Hu=human; N=total number of sample; NH=normal human; NHP=normal human plasma; NHS=normal human serum; RA=rheumatoid arthritis; %=number of samples>lower limit of quantitation (LLOQ)/N×100.

### DETAILED DESCRIPTION OF EMBODIMENTS OF THE INVENTION

### I. Definitions

The term “about” as used herein refers to the usual error range for the respective value readily known to the skilled person in this technical field. Reference to “about” a value or parameter herein includes (and describes) embodiments that are directed to that value or parameter per se. In some embodiments, the term about refers to +10% of a recited value.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity-matured” antibody is one with one or more alterations in one or more HVRs and/or framework regions which result in an improvement in the affinity of the antibody for antigen, compared to a parent antibody which does not possess those alteration(s). Preferred affinity-matured antibodies will have nanomolar or even picomolar affinities for the target antigen. Affinity-matured antibodies are produced by procedures known in the art. For example, Marks et al. Bio/Technology 10:779-783, 1992 describes affinity maturation by VH and VL domain shuffling. Random mutagenesis of HVR and/or framework residues is described by: Barbas et al. Proc. Natl. Acad. Sci. USA 91:3809-3813, 1994; Schier et al. Gene 169:147-155, 1995; Yelton et al. J. Immunol. 155:1994-2004, 1995; Jackson et al. J. Immunol. 154(7):3310-3319, 1995; and Hawkins et al. J. Mol. Biol. 226:889-896, 1992.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “interleukin-33 (IL-33),” as used herein, refers to any native IL-33 from any vertebrate source, including mammals such as primates (e.g., humans and cynomolgus monkeys) and rodents (e.g., mice and rats), unless otherwise indicated. IL-33 is also referred to in the art as nuclear factor of high endothelial venules (NF-HEV; see, e.g., Baekkevold et al. Am. J. Pathol. 163(1): 69-79, 2003), DVS27, C9orf26, and interleukin-1 family member 11 (IL-1F11). The term encompasses “full-length,” unprocessed IL-33, as well as any form of IL-33 that results from processing in the cell. Human full-length, unprocessed IL-33 contains 270 amino acids (a.a.) and may also be referred to as IL-33₁₋₂₇₀. Processed forms of human IL-33 include, for example, IL-33₉₅₋₂₇₀, IL-33₉₉₋₂₇₀, IL-33₁₀₉₋₂₇₀, IL-33₁₁₂₋₂₇₀, IL-33₁₋₁₇₈, and IL-33₁₇₉₋₂₇₀ (Lefrançais et al. Proc. Natl. Acad. Sci. 109 (5): 1673-1678, 2012 and Martin, Semin. Immunol. 25:449-457, 2013). In some embodiments, processed forms of human IL-33, e.g., IL-33₉₅₋₂₇₀, IL-33₉₅₋₂₇₀, IL-33₁₀₉₋₂₇₀, or other forms processed by proteases such as calpain, proteinase 3, neutrophil elastase, and cathepsin G may have increased biological activity compared to full-length IL-33. The term also encompasses naturally occurring variants of IL-33, for example, splice variants (e.g., the constitutively active splice variant spIL-33 which lacks exon 3, Hong et al. J. Biol. Chem. 286(22):20078-20086, 2011) or allelic variants. IL-33 may be present within a cell (e.g., within the nucleus) or as a secreted cytokine form. Full-length IL-33 protein contains a helix-turn-helix DNA-binding motif including nuclear localization sequence (a.a. 1-75 of human IL-33), which includes a chromatin binding motif (a.a. 40-58 of human IL-33). Forms of IL-33 that are processed and secreted lack these N-terminal motifs. The amino acid sequence of an exemplary human IL-33 can be found, for example, under UniProtKB accession number O95760. In some embodiments, IL-33 in vivo may be in an oxidized and/or reduced form. In certain embodiments, reduced IL-33 can be prepared by incubating IL-33 (e.g., human IL-33 (hIL-33)) with a reducing agent for a period of time. In some embodiments, reducing agents that can be used to reduce IL-33 (e.g., hIL-33) or stabilize reduced IL-33 (e.g., hIL-33) include, but are not limited to, dithiothreitol (DTT; CAS #: 3483 Dec. 3) and tris (2-carboxyethyl) phosphine (TCEP; CAS #: 51805-45-9). In certain embodiments, reduced IL-33 binds to ST2. In certain embodiments, reduced IL-33 binds to ST2 with higher affinity than oxidized IL-33. In some embodiments, reduced IL-33 differs from oxidized IL-33 in its secondary structure and in the absence of disulfide bonds. In certain embodiments, reduced IL-33 does not comprise C208-C259 and C227-C232 disulfide bonds between the indicated cysteines. In particular embodiments, the disulfide bonds (e.g., at C208-C259 and C227-C232) in oxidized IL-33prevents its binding to ST2. In some embodiments, “unbound IL-33” refers to IL-33 that is not bound to ST2.

By “IL-33 axis” is meant a nucleic acid (e.g., a gene or mRNA transcribed from the gene) or polypeptide that is involved in IL-33 signal transduction. For example, the IL-33 axis may include the ligand IL-33, a receptor (e.g., ST2 and/or IL-1RAcP), adaptor molecules (e.g., MyD88), or proteins that associate with receptor molecules and/or adaptor molecules (e.g., kinases, such as interleukin-1 receptor-associated kinase 1 (IRAK1) and interleukin-1 receptor-associated kinase 4 (IRAK4), or E3 ubiquitin ligases, such as TNF receptor associated factor 6 (TRAF6)).

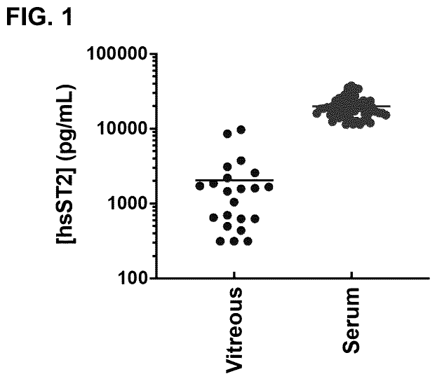

The terms “interleukin 1 receptor-like 1 (IL1RL1)” and “ST2,” used interchangeably herein, refer to any native ST2 from any vertebrate source, including mammals such as primates (e.g., humans) and rodents (e.g., mice and rats), unless otherwise indicated. ST2 is also referred to in the art as DER4, T1, and FIT-1. The term encompasses “full-length,” unprocessed ST2, as well as any form of ST2 that results from processing in the cell. At least four isoforms of ST2 are known in the art, including soluble (sST2, also known as IL1RL1-a) and transmembrane (ST2L, also known as IL1RL1-b), which arise from differential mRNA expression from a dual promoter system, and ST2V and ST2LV, which arise from alternative splicing, as described below. In some examples, ST2, including sST2, maybe human ST2 or human sST2 (hsST2), respectively. The domain structure of ST2L includes three extracellular immunoglobulin-like C2 domains, a transmembrane domain, and a cytoplasmic Toll/Interleukin-1 receptor (TIR) domain. sST2 lacks the transmembrane and cytoplasmic domains contained within ST2L and includes a unique 9 amino acid (a.a.) C-terminal sequence (see, e.g., Kakkar et al. Nat. Rev. Drug Disc. 7:827-840, 2008). sST2 can function as a decoy receptor to inhibit soluble IL-33. The term also encompasses naturally occurring variants of ST2, e.g., splice variants (e.g., ST2V, which lacks the third immunoglobulin motif and has a unique hydrophobic tail, and ST2LV, which lacks the transmembrane domain of ST2L) or allelic variants (e.g., variants that are protective against asthma risk or that confer asthma risk as described herein). The amino acid sequence of an exemplary human ST2 can be found, for example, under UniProtKB accession number Q01638. ST2 is a part of the IL-33 receptor along with the co-receptor protein IL-1RAcP. Binding of IL-33 to ST2 and the co-receptor interleukin-1 receptor accessory protein (IL-1RAcP) forms a 1:1:1 ternary signaling complex to promote downstream signal transduction, as depicted in FIG. 1A (see, e.g., Lingel et al. Structure 17(10):1398-1410, 2009, and Liu et al. Proc. Natl. Acad. Sci. 110(37):14918-14924, 2013). In some embodiments, “IL-1 receptor accessory protein” or “IL-1RAcP” is a co-receptor of ST2.

An “IL-33 axis binding antagonist” refers to a molecule that inhibits the interaction of an IL-33 axis binding partner with one or more of its binding partners. As used herein, an IL-33 axis binding antagonist includes IL-33 binding antagonists, ST2 binding antagonists, and IL1RAP binding antagonists. Exemplary IL-33 axis binding antagonists include anti-IL-33 antibodies and antigen-binding fragments thereof (e.g., anti-IL-33 antibodies such as ANB-020 (AnaptysBio, Inc.) or any of the antibodies described in US2021-0284725A1, U.S. Pat. No. 10,093,730, EP1725261, U.S. Pat. No. 8,187,596, WO2011031600, WO2014164959,WO2015099175, WO2015106080, WO2016077381, or WO2021183849, which are each incorporated herein by reference in their entirety); polypeptides that bind IL-33 and/or its receptor (ST2 and/or IL-1RAcP) and block ligand-receptor interaction (e.g., ST2-Fc proteins, such as those described in WO 2014/152195, which is herein incorporated by reference in its entirety; immunoadhesins, peptibodies, and soluble ST2, or derivatives thereof); anti-IL-33 receptor antibodies (e.g., anti-ST2 antibodies, for example, astegolimab (also known as MSTT1041A), AMG-282 (Amgen) or STLM15 (Janssen) or any of the anti-ST2 antibodies described in WO 2013/173761 and WO 2013/165894, which are each incorporated herein by reference in their entirety; or ST2-Fc proteins, such as those described in WO 2013/173761; WO 2013/165894; or WO 2014/152195, which are each incorporated herein by reference in their entirety); and IL-33 receptor antagonists, such as small molecule inhibitors, aptamers that bind IL-33, and nucleic acids that hybridize under stringent conditions to IL-33 axis nucleic acid sequences (e.g., short interfering RNAs (siRNA) or clustered regularly interspaced short palindromic repeat RNAs (CRISPR-RNA or crRNA), including single guide RNAs (sgRNAs) having a crRNA and tracrRNA sequence as described in Mali et al. (Science. 339:823-26, 2013), which is incorporated herein by reference in its entirety).

The terms “anti-IL-33 antibody,” an “antibody that binds to IL-33,” and “antibody that specifically binds IL-33” refer to an antibody that is capable of binding IL-33 with sufficient affinity such that the antibody is useful as a diagnostic and/or therapeutic agent in targeting IL-33. In one embodiment, the extent of binding of an anti-IL-33 antibody to an unrelated, non-IL-33 protein is less than about 10% of the binding of the antibody to IL-33 as measured, e.g., by a radioimmunoassay (RIA). In certain embodiments, an antibody that binds to IL-33 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In some embodiments, the anti-IL-33 antibody binds to IL-33 with a K_{D} between 0.01 nM and 1 nM. In some embodiments, the anti-IL-33 antibody binds to IL-33 with a K_{D} between 0.1 nM and 0.5 nM. In some embodiments, an anti-IL-33 antibody binds to oxidized and reduced forms of IL-33. In certain embodiments, the anti-IL-33 antibody binds to reduced IL-33. In certain embodiments, the anti-IL-33 antibody binds to oxidized IL-33 and to reduced IL-33 with within two-fold K_{D} (e.g., within one-and-a-half-fold or within one-fold K_{D}). In certain embodiments, the anti-IL-33 antibody binds to oxidized IL-33 and to reduced IL-33 with substantially the same K_{D}. In certain embodiments, an anti-IL-33 antibody binds to an epitope of IL-33 that is conserved among IL-33 from different species. In certain embodiments, the anti-IL-33 antibody binds to mammalian IL-33. In particular embodiments, the anti-IL-33 antibody binds to human IL-33 (hIL-33). In certain embodiments, an anti-IL-33 antibody binds to IL-33 bound to soluble interleukin 1 receptor like 1 (sST2) protein and to IL-33 not bound to sST2 (e.g. unbound IL-33) with within two-fold K_{D} (e.g., within one-and-a-half-fold or within one-fold K_{D}). In certain embodiments, an anti-IL-33 antibody binds to IL-33 bound to sST2 protein with substantially the same K_{D} as to IL-33 not bound to sST2.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al. Protein Eng. 8(10):1057-1062, 1995); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (C_{H}1). Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the C_{H}1 domain including one or more cysteines from the antibody hinge region. Fab'-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the human Fc region or constant region is according to the EU numbering system, also called the EU index, as described in Kabat et al. Sequences of Proteins of Immunological Interest, 5th Ed. Public Health Service, National Institutes of Health, Bethesda, MD, 1991.

“Fv” consists of a dimer of one heavy-and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three Hs specific for an antigen) has the ability to recognize and bind antigen, although often at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315, 1994.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., a fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al. Proc. Natl. Acad. Sci. USA 90:6444-6448, 1993.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

A “blocking” antibody or an “antagonist” antibody is one which inhibits or reduces biological activity of the antigen it binds. Certain blocking antibodies or antagonist antibodies substantially or completely inhibit the biological activity of the antigen.

A “cell-based blocking assay” refers to an assay in which the ability of an antibody to inhibit or reduce the biological activity of the antigen it binds can be measured. For example, a cell-based assay can be used to measure the concentration of antibody required to inhibit a specific biological or biochemical function. In some embodiments, the half maximal inhibitory concentration (IC50) and/or 90% inhibitory concentration (IC90) of an antibody (e.g., an anti-IL-33 antibody disclosed herein) is measured using a cell-based blocking assay. In some embodiments, the cell-based blocking assay is used to determine whether an antibody blocks the interaction between a ligand (e.g., IL-33) and its receptor (e.g., ST2 and/or the coreceptor IL-1RAcP). An exemplary cell-based blocking assay for IL-33 is provided, e.g., in Example 2B of U.S. Pat. No. 10,093,730. Additional exemplary cell-based blocking assays for IL-33 are provided, for example, in Example 8 of U.S. Pat. No. 10,093,730, including primary natural killer (NK) cell assays and primary basophil cell assays.

An “immune-polymerase chain reaction” or “iPCR” assay refers to an assay in which the presence or level of a target molecule (e.g., a target biomolecule, e.g., a protein) is detected by an antibody-DNA conjugate. In some embodiments, an iPCR assay uses a first antibody conjugated to a biotin and a second antibody conjugated to a nucleic acid moiety, wherein the first and second antibodies bind to the same target antigen. Both the first and second antibodies are mixed with a solution containing the target antigen, and the resulting mixture is incubated together, e.g., within a PCR well. PCR amplification is then used to detect the presence of the nucleic acid moiety, and thereby indicate the detection of the target antigen. In some embodiments, the iPCR assay is used to detect IL-33 (e.g., human IL-33, e.g., reduced IL-33). In some embodiments, the first and second antibodies are anti-IL-33 antibodies. In some embodiments, the first and second antibodies share 90%, 95%, 96%, 97%, 98%, 99%, or 100% amino acid sequence identity. In some embodiments, the first and second antibodies do not share the same amino acid sequence (e.g., the amino acid sequences of the first and second antibodies share no more than 1%, 2%, 3%, 4%, 5%, 10%, 15%, or 20% amino acid sequence identity). Examples of iPCR assays are described in Sano T et. al., Science 1992, 258:120-122 and Niemeyer et al., Trends Microbiol. 2017, 8:65.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2 (e.g., IgG2a or IgG2b), igG3,IgG4, IgA1, and IgA2. The heavy chain constant domains that correspond to the different classes of immunoglobulins are called α, δ, ε, γ, and μ, respectively.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch et al. Annu. Rev. Immunol. 9:457-492, 1991. To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. Proc. Natl. Acad. Sci. USA 95:652-656, 1998.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234, 1997). FcRs are reviewed, for example, in Ravetch et al. Annu. Rev. Immunol. 9:457-492, 1991; Capel et al. Immunomethods 4:25-34, 1994; and de Haas et al. J. Lab. Clin. Med. 126:330-41, 1995. Other FcRs, including those to be identified in the future, are encompassed by the term “FcR” herein. The term also includes the neonatal receptor, FcRn, which is responsible for the transfer of maternal IgGs to the fetus (see, e.g., Guyer et al. J. Immunol. 117:587, 1976; and Kim et al. J. Immunol. 24:249, 1994).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells, and neutrophils; with PBMCs and NK cells being preferred. The effector cells can be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al. J. Immunol. Methods 202:163, 1996, can be performed.

An “epitope” is the portion of the antigen to which the antibody selectively binds. For a polypeptide antigen, the epitope is generally a peptide portion of about 4-15 amino acid residues.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human and/or has been made using any of the techniques for making human antibodies. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD, vols. 1-3, 1991. In one embodiment, for the VL, the subgroup is subgroup kappa III or kappa IV as in Kabat et al. supra. In one embodiment, for the VH, the subgroup is subgroup III as in Kabat et al. supra.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al. Nature 321:522-525, 1986; Riechmann et al. Nature 332:323-329, 1988; and Presta, Curr. Op. Struct. Biol. 2:593-596, 1992.

An “immunoconjugate” is an antibody conjugated to one or more heterologous molecule(s), including but not limited to a label or a cytotoxic agent.

The term “isolated” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., sodium dodecyl sulfate polyacrylamide gel electrophoresis (SDS-PAGE), isoelectric focusing (IEF), or capillary electrophoresis) or chromatographic (e.g., ion exchange or reverse phase HPLC) methods. For a review of methods for assessment of antibody purity, see, for example, Flatman et al. J. Chromatogr. B 848:79-87, 2007. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “multispecific antibody” is used in the broadest sense and specifically covers an antibody comprising a heavy chain variable domain (VH) and a light chain variable domain (VL), where the VHVL unit has polyepitopic specificity (i.e., is capable of binding to two different epitopes on one biological molecule or each epitope on a different biological molecule). Such multispecific antibodies include, but are not limited to, full-length antibodies, antibodies having two or more VL and VH domains, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one embodiment, the multispecific antibody in an IgG1 form binds to each epitope with an affinity of 5 μM to 0.001 pM, 3 μM to 0.001 pM, 1 μM to 0.001 pM, 0.5 μM to 0.001 pM or 0.1 μM to 0.001 pM. “Monospecific” refers to the ability to bind only one epitope.

A “naked antibody” refers to an antibody that is not conjugated to a heterologous moiety (e.g., a cytotoxic moiety) or radiolabel. The naked antibody may be present in a pharmaceutical composition.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁵ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

The term “variable” refers to the fact that certain segments of the variable domains differ extensively in sequence among antibodies. The variable or “V” domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al. supra). Accordingly, the HVR and FR sequences generally appear in the following sequence in VH (or VL): FR1-H1(L1)-FR2-H2(L2)-FR3-H3(L3)-FR4. The constant domains are not involved directly in binding an antibody to an antigen, but exhibit various effector functions, such as participation of the antibody in antibody dependent cellular cytotoxicity (ADCC).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3 (CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al. supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al. supra). The “EU numbering system” or “EU index” is generally used when referring to a residue in an immunoglobulin heavy chain constant region (e.g., the EU index reported in Kabat et al. supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

As used herein, “administering” is meant a method of giving a dosage of a compound (e.g., an IL-33 axis binding antagonist provided herein (e.g., anti-IL-33 antibody) or a nucleic acid encoding an anti-IL-33 antibody provided herein) or a composition (e.g., a pharmaceutical composition, e.g., a pharmaceutical composition including an anti-IL-33 antibody provided herein) to a subject. The compositions utilized in the methods described herein can be administered, for example, intravitreally, intramuscularly, intravenously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, orally, topically, transdermally, periocularly, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered, and the severity of the condition, disease, or disorder being treated).

The term “asthma” refers herein to a disorder characterized by variable and recurring symptoms, reversible airflow obstruction (e.g., by bronchodilator), and bronchial hyper-responsiveness, which may or may not be associated with underlying inflammation. Asthma may therefore be inflammatory/inflamed asthma or non-inflammatory/non-inflamed asthma. Examples of asthma include allergic asthma, exercise-induced asthma, aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, and other asthmas as mentioned in Bousquet et al. J. Allergy Clin. Immunol. 126 (5): 926-938, 2010.

A “disorder” or “disease” is any condition that would benefit from treatment (e.g., with an IL-33 axis binding antagonist provided herein). For example, a disorder may be an IL-33-mediated disorder. This includes chronic and acute disorders or diseases including those pathological conditions which predispose the mammal to the disorder in question. Examples of disorders to be treated herein include IL-33-mediated disorders (e.g., asthma, allergic rhinitis, atopic dermatitis, and fibrosis (e.g., pulmonary fibrosis, e.g., idiopathic pulmonary fibrosis)).

The term “IL-33-mediated disorder,” as used herein, refers to any disorder or condition mediated by, or associated with, the IL-33 axis. In some embodiments, IL-33-mediated disorders are associated with excess IL-33 levels or activity in which atypical symptoms may manifest due to the levels or activity of IL-33 locally and/or systemically in the body. Exemplary IL-33-mediated disorders include ocular disorders, inflammatory conditions, immune disorders, fibrotic disorders, eosinophilic disorders, infections, pain, central nervous system disorders, and solid tumors. IL-33-mediated disorders are described, for example, in Liew et al. Nature Reviews Immunology 10:103-110, 2010, which is incorporated herein by reference in its entirety.

The term “ocular disorder,” as used herein, includes any ocular disorder (also referred to interchangeably herein as “ocular condition”) associated with pathological angiogenesis and/or atrophy. An ocular disorder may be characterized by altered or unregulated proliferation and/or invasion of new blood vessels into the structures of ocular tissues such as the retina or cornea. An ocular disorder may be characterized by atrophy of retinal tissue (photoreceptors and the underlying retinal pigment epithelium (RPE) and choriocapillaris). Non-limiting ocular disorders include, for example, AMD (e.g., wet AMD, dry AMD, intermediate AMD, advanced AMD, and geographic atrophy (GA)), macular degeneration, macular edema, diabetic macular edema (DME) (e.g., focal, non-center DME and diffuse, center-involved DME), retinopathy, diabetic retinopathy (DR) (e.g., proliferative DR (PDR), non-proliferative DR (NPDR), and high-altitude DR), other ischemia-related retinopathies, ROP, retinal vein occlusion (RVO) (e.g., central (CRVO) and branched (BRVO) forms), choroidal neovascularization (CNV) (e.g., myopic CNV), corneal neovascularization, diseases associated with corneal neovascularization, retinal neovascularization, diseases associated with retinal/choroidal neovascularization, central serous retinopathy (CSR), pathologic myopia, von Hippel-Lindau disease, histoplasmosis of the eye, familial exudative vitreoretinopathy (FEVR), Coats' disease, Norrie Disease, retinal abnormalities associated with osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, including but not limited to CMV retinitis, ocular melanoma, retinal blastoma, conjunctivitis (e.g., infectious conjunctivitis and non-infectious (e.g., allergic) conjunctivitis), Leber congenital amaurosis (also known as Leber's congenital amaurosis or LCA), uveitis (including infectious and non-infectious uveitis), choroiditis (e.g., multifocal choroiditis), ocular histoplasmosis, blepharitis, dry eye, traumatic eye injury, Sjögren's disease, and other ophthalmic diseases wherein the disease or disorder is associated with ocular neovascularization, vascular leakage, and/or retinal edema or retinal atrophy. Additional exemplary ocular disorders include retinoschisis (abnormal splitting of the retina neurosensory layers), diseases associated with rubeosis (neovascularization of the angle) and diseases caused by the abnormal proliferation of fibrovascular or fibrous tissue, including all forms of proliferative vitreoretinopathy.

Exemplary diseases associated with corneal neovascularization include, but are not limited to, epidemic keratoconjunctivitis, vitamin A deficiency, contact lens overwear, atopic keratitis, superior limbic keratitis, terygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegener's sarcoidosis, scleritis, Stevens-Johnson syndrome, pemphigoid radial keratotomy, and corneal graph rejection.

Exemplary diseases associated with choroidal neovascularization and defects in the retina vasculature, including increased vascular leak, aneurisms and capillary drop-out include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, retina edema (including macular edema), Eales disease, Behcet's disease, infections causing retinitis or choroiditis (e.g., multifocal choroiditis), presumed ocular histoplasmosis, Best's disease (vitelliform macular degeneration), myopia, optic pits, pars planitis, retinal detachment (e.g., chronic retinal detachment), hyperviscosity syndromes, toxoplasmosis, trauma, and post-laser complications.

Exemplary diseases associated with atrophy of retinal tissues (photoreceptors and the underlying RPE) include, but are not limited to, atrophic or nonexudative AMD (e.g., geographic atrophy or advanced dry AMD), macular atrophy (e.g., atrophy associated with neovascularization and/or geographic atrophy), diabetic retinopathy, Stargardt's disease, Sorsby Fundus Dystrophy, retinoschisis, and retinitis pigmentosa.

Exemplary inflammatory conditions include asthma (e.g., allergic asthma, exercise-induced asthma, aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, etc.), airway inflammation, airway hyperreactivity, airway hyperresponsiveness, rhinosinusitis, rhinosinusitis with polyps, nasal polyposis, arthritis (e.g., osteoarthritis, rheumatoid arthritis, collagen-induced arthritis, arthritic joints as a result of injury, etc.), eosinophilic inflammation, mast cell-mediated inflammatory diseases, sepsis, septic shock, seronegative enthesopathy and arthropathy (SEA) syndrome, osteoporosis, eosinophilic esophagitis, scleroderma, dermatitis, atopic dermatitis, allergic rhinitis, bullous pemphigoid, chronic urticaria, cartilage inflammation, polymyalgia rheumatic, polyarteritis nodosa, Wegener's granulomatosis, Behcet's disease, myolitis, polymyolitis, dermatomyolitis, dermatomyositis, vasculitis, arteritis, diabetic nephropathy, interstitial cystitis, graft versus host disease (GVHD), gastrointestinal inflammatory conditions (e.g., inflammatory bowel disease (IBD), ulcerative colitis (UC), Crohn's disease (CD), colitis (e.g., colitis caused by environmental insults (e.g., caused by or associated with a therapeutic regimen, such as chemotherapy, radiation therapy, etc.), infectious colitis, ischemic colitis, collagenous or lymphocytic colitis, necrotizing enterocolitis, colitis in conditions such as chronic granulomatous disease or celiac disease, food allergies, gastritis, infectious gastritis or enterocolitis (e.g., Helicobacter pylori-infected chronic active gastritis), and other forms of gastrointestinal inflammation caused by an infectious agent), and inflammatory pulmonary conditions (e.g., chronic obstructive pulmonary disease (COPD), eosinophilic pulmonary inflammation, infection-induced pulmonary conditions (including those associated with viral (e.g., influenza, parainfluenza, rotavirus, human metapneumovirus, and respiratory syncytial virus), bacterial, fungal (e.g., Aspergillus), parasitic, or prion infection, allergen-induced pulmonary conditions, pollutant-induced pulmonary conditions (e.g., asbestosis, silicosis, or berylliosis), gastric aspiration-induced pulmonary conditions, immune dysregulation, inflammatory conditions with genetic predisposition such as cystic fibrosis, physical trauma-induced pulmonary conditions (e.g., ventilator injury), emphysema, bronchitis, sarcoidosis, histiocytosis, lymphangiomyomatosis, acute lung injury, acute respiratory distress syndrome, chronic lung disease, bronchopulmonary dysplasia, pneumonia (e.g., community-acquired pneumonia, nosocomial pneumonia, ventilator-associated pneumonia, viral pneumonia, bacterial pneumonia, and severe pneumonia), airway exacerbations, and acute respiratory distress syndrome (ARDS)).

Exemplary immune disorders include those mediated at least in part by mast cells, such as asthma (e.g., allergic asthma), eczema, itch, allergy, atopic allergy, anaphylaxis, anaphylactic shock, allergic bronchopulmonary aspergillosis, allergic rhinitis, allergic conjunctivitis, as well as autoimmune disorders including rheumatoid arthritis, juvenile rheumatoid arthritis, psoriatic arthritis, pancreatitis, psoriasis, plaque psoriasis, guttate psoriasis, inverse psoriasis, pustular psoriasis, erythrodermic psoriasis, paraneoplastic autoimmune diseases, autoimmune hepatitis, bullous pemphigoid, myasthenia gravis, inflammatory bowel disease, Crohn's disease, ulcerative colitis, celiac disease, thyroiditis (e.g., Graves' disease), Sjogren's syndrome, Guillain-Barre disease, Raynaud's phenomenon, Addison's disease, liver diseases (e.g., primary biliary cirrhosis, primary sclerosing cholangitis, non-alcoholic fatty liver disease, and non-alcoholic steatohepatitis), and diabetes (e.g., type I diabetes).

As used herein, the terms “fibrotic disorder” and “fibrosis” refer to conditions involving formation of excess fibrous connective tissue in an organ or tissue. Exemplary fibrotic disorders include lung fibrosis, liver fibrosis (e.g., fibrosis associated with cirrhosis (e.g., alcohol-induced cirrhosis, viral-induced cirrhosis, post-hepatitis C cirrhosis, and primary biliary cirrhosis), schistosomiasis, cholangitis (e.g., sclerosing cholangitis), and autoimmune-induced hepatitis), kidney fibrosis (e.g., tubulointerstitial fibrosis, scleroderma, diabetic nephritis, and glomerular nephritis), dermal fibrosis (e.g., scleroderma, hypertrophic and keloid scarring, nephrogenic fibrosing dermatopathy, and burns), myelofibrosis, neurofibromatosis, fibroma, intestinal fibrosis, and fibrotic adhesions resulting from surgical procedures), heart fibrosis (e.g., fibrosis associated with myocardial infarction), vascular fibrosis (e.g., fibrosis associated with postangioplasty arterial restenosis and atherosclerosis), eye fibrosis (e.g., fibrosis associated with post-cataract surgery, proliferative vitreoretinopathy, and retro-orbital fibrosis), and bone marrow fibrosis (e.g., idiopathic myelofibrosis and drug-induced myelofibrosis). The fibrosis can be organ-specific or systemic (e.g., systemic sclerosis and fibrosis associated with GVHD).

Examples of lung fibrosis include, for example, lung or pulmonary fibrosis associated with idiopathic pulmonary fibrosis, fibrosis with collagen vascular disease, Hermansky-Pudlak syndrome, adult respiratory distress syndrome, nonspecific interstitial pneumonia, respiratory bronchiolitis, sarcoidosis, histiocytosis X, bronchiolitis obliterans, and cryptogenic organizing pneumonia. In one embodiment, the lung fibrosis is idiopathic pulmonary fibrosis.

As used herein, an “eosinophilic disorder” is a disorder associated with excess eosinophil numbers in which atypical symptoms may manifest due to the levels or activity of eosinophils locally or systemically in the body. Eosinophilic disorders include but are not limited to, asthma (including aspirin sensitive asthma, atopic asthma, and severe asthma), eosinophilic inflammation, atopic dermatitis, allergic rhinitis (including seasonal allergic rhinitis), non-allergic rhinitis, chronic eosinophilic pneumonia, allergic bronchopulmonary aspergillosis, celiac disease, Churg-Strauss syndrome (periarteritis nodosa plus atopy), eosinophilic myalgia syndrome, hypereosinophilic syndrome, edematous reactions including episodic angiodema, helminth infections, where eosinophils may have a protective role, onchocercal dermatitis, eosinophil-associated gastrointestinal disorders (EGIDs), including but not limited to, eosinophilic esophagitis, eosinophilic gastritis, eosinophilic gastroenteritis, eosinophilic enteritis and eosinophilic colitis, nasal micropolyposis and polyposis, aspirin intolerance, and obstructive sleep apnea. Eosinophil-derived secretory products have also been associated with the promotion of angiogenesis and connective tissue formation in tumors and the fibrotic responses seen in conditions such as chronic asthma, Crohn's disease, scleroderma and endomyocardial fibrosis (Munitz et al. Allergy 59:268-275, 2004; Adamko et al. Allergy 60:13-22, 2005; Oldhoff et al. Allergy 60:693-696, 2005). Other examples include cancer (e.g., glioblastoma (such as glioblastoma multiforme) and non-Hodgkin's lymphoma (NHL)), atopic dermatitis, allergic rhinitis, inflammatory bowel disease, fibrosis (e.g., pulmonary fibrosis (e.g., idiopathic pulmonary fibrosis (IPF) and pulmonary fibrosis secondary to sclerosis) and hepatic fibrosis), and COPD.

The above list is not all-inclusive, and it will be understood by the skilled artisan that a disease or disorder may fall within various categories. For example, asthma can be categorized in some instances as both an inflammatory disorder and immune disorder and considered by some clinicians to be an autoimmune disorder.

The term “ST2 binding antagonist” refers to a molecule that inhibits the interaction of an ST2 with IL-33, IL1RAP, and/or a second ST2 molecule. The ST2 binding antagonist may be a protein, such as an “ST2-Fc protein” that includes an IL-33-binding domain (e.g., all or a portion of an ST2 or IL1RAcP protein) and a multimerizing domain (e.g., an Fc portion of an immunoglobulin, e.g., an Fc domain of an IgG selected from the isotypes IgG1, IgG2, IgG3, and IgG4, as well as any allotype within each isotype group), which are attached to one another either directly or indirectly through a linker (e.g., a serine-glycine (SG) linker, glycine-glycine (GG) linker, or variant thereof (e.g., a SGG, a GGS, an SGS, or a GSG linker)), and includes, but is not limited to, ST2-Fc proteins and variants thereof described in WO 2013/173761, WO 2013/165894, and WO 2014/152195, which are each incorporated herein by reference in their entirety. In some embodiments, a ST2 binding antagonist may be an anti-ST2 antibody, for example, astegolimab, AMG-282 (Amgen) or STLM15 (Janssen) or any of the anti-ST2 antibodies described in WO 2013/173761 and WO 2013/165894.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. The host cell may be an “isolated host cell,” which refers to a host cell that has been separated from a component of its natural environment. In some embodiments, host cells may be human or rate in origin.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header. The amino acid sequences described herein are contiguous amino acid sequences unless otherwise specified.

The term “package insert” is used to refer to instructions customarily included in commercial packages of diagnostic or therapeutic products, that contain information about the indications, usage, dosage, administration, combination therapy, contraindications and/or warnings concerning the use of such products.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

By “reduce or inhibit” is meant the ability to cause an overall decrease preferably of 20% or greater, more preferably of 50% or greater, and most preferably of 75%, 85%, 90%, 95%, or greater. Reduce or inhibit can refer to the symptoms of the disorder being treated, the presence or size of metastases, and/or the size of the primary tumor.

A “subject” is a vertebrate, preferably a mammal, more preferably a human. Mammals include, but are not limited to, farm animals (such as cows, and sheep), sport animals, pets (such as cats, dogs and horses), primates (e.g., humans and non-human primates such as monkeys), and rodents (e.g., mice and rats). In some embodiments, the anti-IL-33 antibodies provided herein are used to determine the presence or level of IL-33 in a subject, particularly, the presence or level of human IL-33 in a human subject.

The term “therapeutically effective amount” refers to an amount of a compound (e.g., an IL-33 axis binding antagonist, e.g., an anti-IL-33 antibody, provided herein (including an antibody fragment, such an Fab fragment) or a nucleic acid encoding an anti-IL-33 antibody provided herein) or a composition (e.g., a pharmaceutical composition, e.g., a pharmaceutical composition including an IL-33 axis binding antagonist, e.g., an anti-IL-33 antibody, provided herein) to treat a disease or disorder in a subject. In the case of an IL-33-mediated disorder, the therapeutically effective amount of IL-33 axis binding antagonist, e.g., the antibody or antibody fragment (e.g., an anti-IL-33 antibody, including bispecific anti-IL-33 antibodies that bind to IL-33 and a second biological molecule, e.g., IL-13, e.g., bispecific anti-IL-33/anti-IL-13 antibodies), may ameliorate or treat the disease, or prevent, reduce, ameliorate, or treat symptoms associated with the disease. In the case of a proliferative disease (e.g., a solid tumor), the therapeutically effective amount of the antibody or antibody fragment may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the antibody or antibody fragment may prevent growth and/or kill existing cancer cells, it may be cytostatic and/or cytotoxic. For cancer therapy, efficacy in vivo can, for example, be measured by assessing the duration of survival, time to disease progression (TTP), duration of disease free survival (DFS), duration of progression free survival (PFS), the response rates (RR), duration of response, and/or quality of life.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies disclosed herein are used to delay development of a disease or to slow the progression of a disease. A patient may be successfully “treated” for asthma if, for example, after receiving an asthma therapy, the patient shows observable and/or measurable reduction in or absence of one or more of the following: recurrent wheezing, coughing, trouble breathing, chest tightness, symptoms that occur or worsen at night, symptoms that are triggered by cold air, exercise or exposure to allergens.

“Tumor”, as used herein, refers to all neoplastic cell growth and proliferation, whether malignant or benign, and all pre-cancerous and cancerous cells and tissues.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”). In general, expression vectors of utility in recombinant DNA techniques are often in the form of plasmids. In the present specification, “plasmid” and “vector” may be used interchangeably.

The term “detection” includes any means of detecting, including direct and indirect detection.

The term “diagnosis” is used herein to refer to the identification or classification of a molecular or pathological state, disease or condition (e.g., an IL-33-mediated disorder). For example, “diagnosis” may refer to identification of a particular type of an IL-33 mediated disorder. “Diagnosis” may also refer to the classification of a particular subtype of an IL-33-mediated disorder, for instance, by histopathological criteria, or by molecular features (e.g., a subtype characterized by expression of one or a combination of biomarkers (e.g., particular genes or proteins encoded by said genes)).

The terms “level of expression” or “expression level” in general are used interchangeably and generally refer to the amount of a polynucleotide, mRNA, or an amino acid product or protein in a biological sample. “Expression” generally refers to the process by which gene-encoded information is converted into the structures present and operating in the cell. Therefore, according to the present disclosure, “expression” of a gene (e.g., the IL-33 gene) may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. In some embodiments, “expression level” refers to amount of a protein (e.g., IL-33) in a biological sample as determined using methods known in the art or described herein, including but not limited to immunohistochemistry (IHC), immunoblotting (e.g., Western blotting), immunofluorescence (IF), flow cytometry, for example Fluorescence-Activated Cell Sorting (FACS™), Enzyme-Linked Immunosorbant Assay (ELISA), or immune-polymerase chain reaction (iPCR). “Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker).

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker). In some embodiments, reduced expression is little or no expression.

The word “label” when used herein refers to a compound or composition that is conjugated or fused directly or indirectly to a reagent such as a polynucleotide probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

A “reference sample,” “reference cell,” “reference tissue,” “reference level,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

### II. Compositions and Methods

In one aspect, the invention is based, at least in part, on improved antibodies that bind to IL-33.The antibodies provided herein have unexpectedly advantageous properties, including high affinity, particularly to IL-33 in a reduced state (e.g., reduced IL-33; e.g., reduced human IL-33 (hIL-33)) or to IL-33 associated with Interleukin 1 Receptor-Like 1 (ST2; e.g., soluble ST2 (sST2); e.g., human sST2 (hsST2)). For example, the antibodies disclosed herein may specifically bind to reduced IL-33 (e.g., hIL-33) with substantially the same binding affinity (e.g., K_{D}) as to oxidized IL-33 (e.g., hIL-33), as assessed using surface plasmon resonance (SPR), ELISA, or immune-polymerase chain reaction (iPCR). Furthermore, the antibodies disclosed herein may specifically bind to IL-33 (e.g., hIL-33) bound to ST2 (e.g., sST2 or hsST2) with substantially the same binding affinity as to unbound IL-33 (e.g., hIL-33) as assessed using SPR, ELISA, or iPCR. Moreover, the antibodies provided herein also bind to human and cynomolgus monkey (cyno) IL-33 with high affinity, e.g., as assessed using SPR, ELISA, or iPCR. The unexpectedly favorable properties described above (including ability to bind to both reduced and oxidized IL-33 and both unbound IL-33 and IL-33 bound to ST2) are particularly advantageous in the context of biochemical assays (e.g., for detecting the presence of IL-33 in physiological conditions with high sensitivity; e.g., using ELISA or iPCR). For example, without wishing to be bound by any theory, the antibodies disclosed herein may bind to physiologically relevant forms of hIL-33 with sub-nanomolar K_{D}.

### A. Exemplary Anti-IL-33 Antibodies

In some embodiments, the invention provides isolated antibodies or antigen-binding fragments thereof that bind to IL-33. In some embodiments, an antigen-binding molecule of the present invention comprises at least one, at least two, at least three, at least four, at least five, or all six CDRs as illustrated in Table 1. In some instances, the antibody or antigen-binding fragment thereof comprises a VH and/or a VL as in Table 1. In some instances, the antibody comprises a HC and/or a LC as illustrated in Table 1.

TABLE 1
Listing of SEQ. ID. NOs. for antigen-
binding molecules against IL-33

| Listing of | CDR (Kabat) | | | | | | VH/VL | | HC/LC | |
|---|---|---|---|---|---|---|---|---|---|---|
| SEQ. ID. NOs. | H1 | H2 | H3 | L1 | L2 | L3 | VH | VL | HC | LC |
| 3F10 | 1 | 2 | 3 | 4 | 5 | 6 | 7 | 8 | 9 | 10 |
| 15.21C7 | 19 | 20 | 21 | 22 | 23 | 24 | 25 | 26 | 27 | 28 |

In one example, provided herein is an anti-IL-33 antibody or antigen-binding fragment thereof that includes at least one, two, three, four, five, or six CDRs selected from: (a) CDR-H1 comprising the amino acid sequence of SQNVH (SEQ ID NO: 1); (b) CDR-H2 comprising the amino acid sequence of RMRFNGDTSYNSTLKS (SEQ ID NO: 2); (c) CDR-H3 comprising the amino acid sequence of QRDNYGSYYFDD (SEQ ID NO: 3); (d) CDR-L1 comprising the amino acid sequence of RASESVLTLLN (SEQ ID NO: 4); (e) CDR-L2 comprising the amino acid sequence of LASHLES (SEQ ID NO: 5); and (f) CDR-L3 comprising the amino acid sequence of QQSWIDPWT (SEQ ID NO: 6), or a combination of one or more of the above CDRs and/or one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 1-6. For example, in some instances, the anti-IL-33 antibody includes at least one, two, three, four, five, or six CDRs selected from: (a) CDR-H1 comprising the amino acid sequence of SQNVH (SEQ ID NO: 1); (b) CDR-H2 comprising the amino acid sequence of RMRFNGDTSYNSTLKS (SEQ ID NO: 2); (c) CDR-H3 comprising the amino acid sequence of QRDNYGSYYFDD (SEQ ID NO: 3); (d) CDR-L1 comprising the amino acid sequence of RASESVLTLLN (SEQ ID NO: 4); (e) CDR-L2 comprising the amino acid sequence of LASHLES (SEQ ID NO: 5); and (f) CDR-L3 comprising the amino acid sequence of QQSWIDPWT (SEQ ID NO: 6). In another example, provided herein is an anti-IL-33 antibody or antigen-binding fragment thereof that includes (a) a VH domain comprising an amino acid sequence having at least 90% sequence identity (e.g., at least 91%, 92%, 93%, 94%, 95%, 96%, 97%, 98%, or 99% sequence identity) to, or the sequence of, the amino acid sequence of QVQLEESGPGLVQPSQTLSLTCTVSGFSLTSQNVHWVRQPPGKGLEWMGRMRFNGDTSYNSTLKSRL SISRDTSKNQVFLRLNSLQTDDTGTYYCARQRDNYGSYYFDDWGQGIMVTVSS (SEQ ID NO: 7); (b) a VL domain comprising an amino acid sequence having at least 90% sequence identity (e.g., at least 91%, 92%, 93%, 94%, 95%, 96%, 97%, 98%, or 99% sequence identity) to, or the sequence of, the amino acid sequence of DTVLTQSPALAVSPGERVTISCRASESVLTLLNWYQQKPGQHPKLLIYLASHLESGVPARFSGRGSGTDF TLTIDPVEADDTASYYCQQSWIDPWTFGGGTTLELK (SEQ ID NO: 8); or (c) a VH domain as in (a) and a VL domain as in (b).

Any of the anti-IL-33 antibodies or antigen-binding fragments thereof provided herein may include one, two, three, or four of the following heavy chain framework regions: an FR-H1 comprising the amino acid sequence of QVQLEESGPGLVQPSQTLSLTCTVSGFSLT (SEQ ID NO: 11); an FR-H2 comprising the amino acid sequence of WVRQPPGKGLEWMG (SEQ ID NO: 12); an FR-H3 comprising the amino acid sequence of RLSISRDTSKNQVFLRLNSLQTDDTGTYYCAR (SEQ ID NO: 13); and an FR-H4 comprising the amino acid sequence of WGQGIMVTVSS (SEQ ID NO: 14), or a combination of one or more of the above FRs and/or one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 11-14. In some instances, the anti-IL-33 antibody may include one, two, three, or four of the following heavy chain framework regions: an FR-H1 comprising the amino acid sequence of QVQLEESGPGLVQPSQTLSLTCTVSGFSLT (SEQ ID NO: 11); an FR-H2 comprising the amino acid sequence of WVRQPPGKGLEWMG (SEQ ID NO: 12); an FR-H3 comprising the amino acid sequence of RLSISRDTSKNQVFLRLNSLQTDDTGTYYCAR (SEQ ID NO: 13); and an FR-H4 comprising the amino acid sequence of WGQGIMVTVSS (SEQ ID NO: 14). In some embodiments, the VH domain comprises the amino acid sequence of SEQ ID NO: 7.

Any of the anti-IL-33 antibodies or antigen-binding fragments thereof provided herein may include one, two, three, or four of the following light chain framework regions: an FR-L1 comprising the amino acid sequence of DTVLTQSPALAVSPGERVTISC (SEQ ID NO: 15); an FR-L2 comprising the amino acid sequence of WYQQKPGQHPKLLIY (SEQ ID NO: 16); an FR-L3 comprising the amino acid sequence of GVPARFSGRGSGTDFTLTIDPVEADDTASYYC (SEQ ID NO: 17); and an FR-L4 comprising the amino acid sequence of FGGGTTLELK (SEQ ID NO: 18), or a combination of one or more of the above FRs and/or one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 15-18. In some instances, the anti-IL-33 antibody may include one, two, three, or four of the following light chain framework regions: an FR-L1 comprising the amino acid sequence of DTVLTQSPALAVSPGERVTISC (SEQ ID NO: 15); an FR-L2 comprising the amino acid sequence of WYQQKPGQHPKLLIY (SEQ ID NO: 16); an FR-L3 comprising the amino acid sequence of GVPARFSGRGSGTDFTLTIDPVEADDTASYYC (SEQ ID NO: 17); and an FR-L4 comprising the amino acid sequence of FGGGTTLELK (SEQ ID NO: 18). In some instances, the VL domain comprises the amino acid sequence of SEQ ID NO: 8.

In another example, provided herein is an anti-IL-33 antibody that includes (a) a heavy chain (HC) comprising an amino acid sequence having at least 90% sequence identity (e.g., at least 91%, 92%, 93%, 94%, 95%, 96%, 97%, 98%, or 99% sequence identity) to, or the sequence of, the amino acid sequence of QVQLEESGPGLVQPSQTLSLTCTVSGFSLTSQNVHWVRQPPGKGLEWMGRMRFNGDTSYNSTLKSRL SISRDTSKNQVFLRLNSLQTDDTGTYYCARQRDNYGSYYFDDWGQGIMVTVSSASTKGPSVFPLAPSSK STSGGTAALGCLVKDYFPEPVTVSWNSGALTSGVHTFPAVLQSSGLYSLSSVVTVPSSSLGTQTYICNVN HKPSNTKVDKKVEPKSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSDGSFFLYSKLTV DKSRWQQGNVFSCSVMHEALHNHYTQKSLSLSPGK (SEQ ID NO: 9); (b) a light chain (LC) comprising an amino acid sequence having at least 90% sequence identity (e.g., at least 91%, 92%, 93%, 94%, 95%, 96%, 97%, 98%, or 99% sequence identity) to, or the sequence of, the amino acid sequence of DTVLTQSPALAVSPGERVTISCRASESVLTLLNWYQQKPGQHPKLLIYLASHLESGVPARFSGRGSGTDF TLTIDPVEADDTASYYCQQSWIDPWTFGGGTTLELKRTVAAPSVFIFPPSDEQLKSGTASVVCLLNNFYPR EAKVQWKVDNALQSGNSQESVTEQDSKDSTYSLSSTLTLSKADYEKHKVYACEVTHQGLSSPVTKSFNR GEC (SEQ ID NO: 10); or (c) a HC as in (a) and a LC as in (b).

For instance, the anti-IL-33 antibody may include (a) CDR-H1 comprising the amino acid sequence of SQNVH (SEQ ID NO: 1); (b) CDR-H2 comprising the amino acid sequence of RMRFNGDTSYNSTLKS (SEQ ID NO: 2); (c) CDR-H3 comprising the amino acid sequence of QRDNYGSYYFDD (SEQ ID NO: 3); (d) CDR-L1 comprising the amino acid sequence of RASESVLTLLN (SEQ ID NO: 4); (e) CDR-L2 comprising the amino acid sequence of LASHLES (SEQ ID NO: 5); and (f) CDR-L3 comprising the amino acid sequence of QQSWIDPWT (SEQ ID NO: 6). In some instances, the anti-IL-33 antibody comprises (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable (VL) domain comprising an amino acid sequence having at least 90% sequence (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b). In some instances, the anti-IL-33 antibody includes the following heavy chain framework regions: an FR-H1 comprising the amino acid sequence of QVQLEESGPGLVQPSQTLSLTCTVSGFSLT (SEQ ID NO: 11); an FR-H2 comprising the amino acid sequence of WVRQPPGKGLEWMG (SEQ ID NO: 12); an FR-H3 comprising the amino acid sequence of RLSISRDTSKNQVFLRLNSLQTDDTGTYYCAR (SEQ ID NO: 13); and an FR-H4 comprising the amino acid sequence of WGQGIMVTVSS (SEQ ID NO: 14). In some instances, the anti-IL-33 antibody includes the following light chain framework regions: an FR-L1 comprising the amino acid sequence of DTVLTQSPALAVSPGERVTISC (SEQ ID NO: 15); an FR-L2 comprising the amino acid sequence of WYQQKPGQHPKLLIY (SEQ ID NO: 16); an FR-L3 comprising the amino acid sequence of GVPARFSGRGSGTDFTLTIDPVEADDTASYYC (SEQ ID NO: 17); and an FR-L4 comprising the amino acid sequence of FGGGTTLELK (SEQ ID NO: 18). In some instances, the anti-IL-33 antibody includes a binding domain comprising (a) a VH domain comprising an amino acid sequence of SEQ ID NO: 7 and (b) a VL domain comprising an amino acid sequence of SEQ ID NO: 8. In some instances, the exemplary anti-IL-33 antibody is 3F10.

For example, provided herein is an isolated antibody that specifically binds IL-33, or an antigen-binding fragment thereof, wherein the antibody comprises (a) a VH domain comprising an amino acid sequence of SEQ ID NO: 7 and (b) a VL domain comprising an amino acid sequence of SEQ ID NO: 8. In some instances, the exemplary anti-IL-33 antibody is 3F10.

In another example, provided herein is an isolated antibody that specifically binds IL-33, or an antigen-binding fragment thereof, wherein the antibody comprises (a) a heavy chain (HC) comprising an amino acid sequence of SEQ ID NO: 9 and (b) a light chain (LC) comprising an amino acid sequence of SEQ ID NO: 10. In some instances, the exemplary anti-IL-33 antibody is 3F10.

In one example, provided herein is an anti-IL-33 antibody or antigen-binding fragment thereof that includes at least one, two, three, four, five, or six CDRs selected from: (a) CDR-H1 comprising the amino acid sequence of DFWMS (SEQ ID NO: 19); (b) CDR-H2 comprising the amino acid sequence of DIKNDGSHTKYAPSLQN (SEQ ID NO: 20); (c) CDR-H3 comprising the amino acid sequence of VGGTYTAYY (SEQ ID NO: 21); (d) CDR-L1 comprising the amino acid sequence of RTSQGINTYLN (SEQ ID NO: 22); (e) CDR-L2 comprising the amino acid sequence of YTSNLES (SEQ ID NO: 23); and (f) CDR-L3 comprising the amino acid sequence of QQYASSPWT (SEQ ID NO: 24), or a combination of one or more of the above CDRs and/or one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 19-24. For example, in some instances, the anti-IL-33 antibody includes at least one, two, three, four, five, or six CDRs selected from: (a) CDR-H1 comprising the amino acid sequence of DFWMS (SEQ ID NO: 19); (b) CDR-H2 comprising the amino acid sequence of DIKNDGSHTKYAPSLQN (SEQ ID NO: 20); (c) CDR-H3 comprising the amino acid sequence of VGGTYTAYY (SEQ ID NO: 21); (d) CDR-L1 comprising the amino acid sequence of RTSQGINTYLN (SEQ ID NO: 22); (e) CDR-L2 comprising the amino acid sequence of YTSNLES (SEQ ID NO: 23); and (f) CDR-L3 comprising the amino acid sequence of QQYASSPWT (SEQ ID NO: 24).

In another example, provided herein is an anti-IL-33 antibody or antigen-binding fragment thereof that includes (a) a VH domain comprising an amino acid sequence having at least 90% sequence identity (e.g., at least 91%, 92%, 93%, 94%, 95%, 96%, 97%, 98%, or 99% sequence identity) to, or the sequence of, the amino acid sequence of EVQLVESGGGLVQPGTSLKLSCVASGFTFSDFWMSWVRQTPGKTMEWIADIKNDGSHTKYAPSLQNRF TISRDNAKNTLYLEVTDVRSEDTATYYCTRVGGTYTAYYWGHGVMVTVSS (SEQ ID NO: 25); (b) a VL domain comprising an amino acid sequence having at least 90% sequence identity (e.g., at least 91%, 92%, 93%, 94%, 95%, 96%, 97%, 98%, or 99% sequence identity) to, or the sequence of, the amino acid sequence of DIQMTQTPSSMPASLGERVTLSCRTSQGINTYLNWYQQKPDGTITPLIHYTSNLESGVPSRFSGSGFGTD YFLTISSLEPEDFAMYFCQQYASSPWTFGGGTKLELK (SEQ ID NO: 26); or (c) a VH domain as in (a) and a VL domain as in (b).

Any of the anti-IL-33 antibodies or antigen-binding fragments thereof provided herein may include one, two, three, or four of the following heavy chain framework regions: an FR-H1 comprising the amino acid sequence of EVOLVESGGGLVQPGTSLKLSCVASGFTFS (SEQ ID NO: 29); an FR-H2 comprising the amino acid sequence of WVRQTPGKTMEWI (SEQ ID NO: 30); an FR-H3 comprising the amino acid sequence of RFTISRDNAKNTLYLEVTDVRSEDTATYYCTR (SEQ ID NO: 31); and an FR-H4 comprising the amino acid sequence of WGHGVMVTVSS (SEQ ID NO: 32), or a combination of one or more of the above FRs and/or one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 29-32. In some instances, the anti-IL-33 antibody may include one, two, three, or four of the following heavy chain framework regions: an FR-H1 comprising the amino acid sequence of EVQLVESGGGLVQPGTSLKLSCVASGFTFS (SEQ ID NO: 29); an FR-H2 comprising the amino acid sequence of WVRQTPGKTMEWI (SEQ ID NO: 30); an FR-H3 comprising the amino acid sequence of RFTISRDNAKNTLYLEVTDVRSEDTATYYCTR (SEQ ID NO: 31); and an FR-H4 comprising the amino acid sequence of WGHGVMVTVSS (SEQ ID NO: 32). In some embodiments, the VH domain comprises the amino acid sequence of SEQ ID NO: 25.

Any of the anti-IL-33 antibodies or antigen-binding fragments thereof provided herein may include one, two, three, or four of the following light chain framework regions: an FR-L1 comprising the amino acid sequence of DIQMTQTPSSMPASLGERVTLSC (SEQ ID NO: 33); an FR-L2 comprising the amino acid sequence of WYQQKPDGTITPLIH (SEQ ID NO: 34); an FR-L3 comprising the amino acid sequence of GVPSRFSGSGFGTDYFLTISSLEPEDFAMYFC (SEQ ID NO: 35); and an FR-L4 comprising the amino acid sequence of FGGGTKLELK (SEQ ID NO: 36), or a combination of one or more of the above FRs and/or one or more variants thereof having at least about 80% sequence identity (e.g., 81%, 82%, 83%, 84%, 85%, 86%, 87%, 88%, 89%, 90%, 91%, 92%, 93%, 94%, 95%, 96%, 97%, 98%, or 99% identity) to any one of SEQ ID NOs: 33-36. In some instances, the anti-IL-33 antibody may include one, two, three, or four of the following light chain framework regions: an FR-L1 comprising the amino acid sequence of DIQMTQTPSSMPASLGERVTLSC (SEQ ID NO: 33); an FR-L2 comprising the amino acid sequence of WYQQKPDGTITPLIH (SEQ ID NO: 34); an FR-L3 comprising the amino acid sequence of GVPSRFSGSGFGTDYFLTISSLEPEDFAMYFC (SEQ ID NO: 35); and an FR-L4 comprising the amino acid sequence of FGGGTKLELK (SEQ ID NO: 36). In some instances, the VL domain comprises the amino acid sequence of SEQ ID NO: 26.

In another example, provided herein is an anti-IL-33 antibody that includes (a) a heavy chain (HC) comprising an amino acid sequence having at least 90% sequence identity (e.g., at least 91%, 92%, 93%, 94%, 95%, 96%, 97%, 98%, or 99% sequence identity) to, or the sequence of, the amino acid sequence of EVQLVESGGGLVQPGTSLKLSCVASGFTFSDFWMSWVRQTPGKTMEWIADIKNDGSHTKYAPSLQNRF TISRDNAKNTLYLEVTDVRSEDTATYYCTRVGGTYTAYYWGHGVMVTVSSAETTAPSVYPLAPGTALKSN SMVTLGCLVKGYFPEPVTVTWNSGALSSGVHTFPAVLQSGLYTLTSSVTVPSSTWSSQAVTCNVAHPAS STKVDKKIVPRECNPCGCTGSEVSSVFIFPPKTKDVLTITLTPKVTCVVVDISQNDPEVRFSWFIDDVEVHT AQTHAPEKQSNSTLRSVSELPIVHRDWLNGKTFKCKVNSGAFPAPIEKSISKPEGTPRGPQVYTMAPPKE EMTQSQVSITCMVKGFYPPDIYTEWKMNGQPQENYKNTPPTMDTDGSYFLYSKLNVKKETWQQGNTFT CSVLHEGLHNHHTEKSLSHSPGK (SEQ ID NO: 27); (b) a light chain (LC) comprising an amino acid sequence having at least 90% sequence identity (e.g., at least 91%, 92%, 93%, 94%, 95%, 96%, 97%, 98%, or 99% sequence identity) to, or the sequence of, the amino acid sequence of DIQMTQTPSSMPASLGERVTLSCRTSQGINTYLNWYQQKPDGTITPLIHYTSNLESGVPSRFSGSGFGTD YFLTISSLEPEDFAMYFCQQYASSPWTFGGGTKLELKKRADAAPTVSIFPPSMEQLTSGGATVVCFVNNF YPRDISVKWKIDGSEQRDGVLDSVTDQDSKDSTYSMSSTLSLTKVEYERHNLYTCEVVHKTSSSPVVKSF NRNEC (SEQ ID NO: 28); or (c) a HC as in (a) and a LC as in (b).

For instance, the anti-IL-33 antibody may include (a) CDR-H1 comprising the amino acid sequence of DFWMS (SEQ ID NO: 19); (b) CDR-H2 comprising the amino acid sequence of DIKNDGSHTKYAPSLQN (SEQ ID NO: 20); (c) CDR-H3 comprising the amino acid sequence of VGGTYTAYY (SEQ ID NO: 21); (d) CDR-L1 comprising the amino acid sequence of RTSQGINTYLN (SEQ ID NO: 22); (e) CDR-L2 comprising the amino acid sequence of YTSNLES (SEQ ID NO: 23); and (f) CDR-L3 comprising the amino acid sequence of QQYASSPWT (SEQ ID NO: 24). In some instances, the anti-IL-33 antibody comprises (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 25; (b) a light chain variable (VL) domain comprising an amino acid sequence having at least 90% sequence (e.g., at least 91%, 92%, 93%, 94%, 95%, 96%, 97%, 98%, or 99% sequence identity) to, or the sequence of, SEQ ID NO: 26; or (c) a VH domain as in (a) and a VL domain as in (b). In some instances, the anti-IL-33 antibody includes the following heavy chain framework regions: an FR-H1 comprising the amino acid sequence of EVQLVESGGGLVQPGTSLKLSCVASGFTFS (SEQ ID NO: 29); an FR-H2 comprising the amino acid sequence of WVRQTPGKTMEWI (SEQ ID NO: 30); an FR-H3 comprising the amino acid sequence of RFTISRDNAKNTLYLEVTDVRSEDTATYYCTR (SEQ ID NO: 31); and an FR-H4 comprising the amino acid sequence of WGHGVMVTVSS (SEQ ID NO: 32). In some instances, the anti-IL-33 antibody includes the following light chain framework regions: an FR-L1 comprising the amino acid sequence of DIQMTQTPSSMPASLGERVTLSC (SEQ ID NO: 33); an FR-L2 comprising the amino acid sequence of WYQQKPDGTITPLIH (SEQ ID NO: 34); an FR-L3 comprising the amino acid sequence of GVPSRFSGSGFGTDYFLTISSLEPEDFAMYFC (SEQ ID NO: 35); and an FR-L4 comprising the amino acid sequence of FGGGTKLELK (SEQ ID NO: 36). In some instances, the anti-IL-33 antibody includes a binding domain comprising (a) a VH domain comprising an amino acid sequence of SEQ ID NO: 25 and (b) a VL domain comprising an amino acid sequence of SEQ ID NO: 26. In some instances, the exemplary anti-IL-33 antibody is 15.21C7.

For example, provided herein is an isolated antibody that specifically binds IL-33, or an antigen-binding fragment thereof, wherein the antibody comprises (a) a VH domain comprising an amino acid sequence of SEQ ID NO: 25 and (b) a VL domain comprising an amino acid sequence of SEQ ID NO: 26. In some instances, the exemplary anti-IL-33 antibody is 15.21C7.

In another example, provided herein is an isolated antibody that specifically binds IL-33, or an antigen-binding fragment thereof, wherein the antibody comprises (a) a heavy chain (HC) comprising an amino acid sequence of SEQ ID NO: 27 and (b) a light chain (LC) comprising an amino acid sequence of SEQ ID NO: 28. In some instances, the exemplary anti-IL-33 antibody is 15.21C7.

Any of the antibodies provided herein may specifically bind IL-33 in an oxidized state (e.g., oxidized IL-33) or IL-33 in a reduced state (e.g., reduced IL-33). In some instances, the antibody specifically binds to reduced IL-33. In certain instances, an anti-IL-33 antibody provided herein binds to oxidized IL-33 and to reduced IL-33 with within two-fold (e.g., within one-and-a-half-fold or within one-fold) K_{D}. In some embodiments, the antibody binds to oxidized IL-33 and to reduced IL-33 with substantially the same K_{D}. In some instances, the antibody binds to reduced IL-33. In some instances, the antibody binds to reduced IL-33 with a K_{D}≤1 nM. In some instances, the antibody binds to reduced IL-33 with a Kp between 0.01 nM and 1 nM (e.g., between 0.01 and 0.9 nM, between 0.01 and 0.8 nM, between 0.01 and 0.7 nM, between 0.01 and 0.6 nM, between 0.01 and 0.5 nM, between 0.05 and 0.9 nM, between 0.1 and 0.9 nM, between 0.2 and 0.9 nM, between 0.3 and 0.9 nM, between 0.2 and 0.6 nM, between 0.3 and 0.5 nM, between 0.3 and 0.4 nM, or between 0.4 and 0.5 nM; e.g., about 0.05 nM, about 0.1 nM, about 0.2 nM, about 0.3 nM, about 0.325 nM, about 0.34 nM, about 0.35 nM, about 0.4 nM, about 0.45 nM, about 0.475 nM, about 0.49 nM, about 0.5 nM, about 0.6 nM, about 0.7 nM, about 0.8 nM, about 0.9 nM, or about 1 nM). In some instances, the antibody binds to reduced IL-33 with a K_{D}≤0.5 nM. In certain instances, the antibody binds to reduced IL-33 with a K_{D} between 0.1 nM and 0.5 nM (e.g., between 0.2 and 0.5 nM, between 0.3 nM and 0.5 nM, between 0.3 nM and 0.4 nM, or between 0.4 nM and 0.5 nM). In certain instances, the antibody binds to reduced IL-33 with a K_{D} of about 0.487 nM. In certain instances, the antibody binds to reduced IL-33 with a K_{D} of about 0.338 nM. Any of the preceding K_{D} values may be determined by surface plasmon resonance (SPR) (see, e.g., Examples 1 and 2).

Any of the antibodies provided herein may specifically bind human or cynomolgus monkey (cyno) IL-33. In some instances, the antibody specifically binds human IL-33. In some instances, the antibody binds to human IL-33 with a K_{D}≤1 nM. In some instances, the antibody binds to human IL-33 with a K_{D} between 0.01 nM and 1 nM (e.g., between 0.01 and 0.9 nM, between 0.01 and 0.8 nM, between 0.01 and 0.7 nM, between 0.01 and 0.6 nM, between 0.01 and 0.5 nM, between 0.05 and 0.9 nM, between 0.1 and 0.9 nM, between 0.2 and 0.9 nM, between 0.3 and 0.9 nM, between 0.2 and 0.6 nM, between 0.3 and 0.5 nM, between 0.3 and 0.4 nM, or between 0.4 and 0.5 nM; e.g., about 0.05 nM, about 0.1 nM, about 0.2 nM, about 0.3 nM, about 0.325 nM, about 0.34 nM, about 0.35 nM, about 0.4 nM, about 0.45 nM, about 0.475 nM, about 0.49 nM, about 0.5 nM, about 0.6 nM, about 0.7 nM, about 0.8 nM, about 0.9 nM, or about 1 nM). In some instances, the antibody binds to human IL-33 with a K_{D}≤0.5 nM. In certain instances, the antibody binds to human IL-33 with a K_{D} between 0.1 nM and 0.5 nM (e.g., between 0.2and 0.5 nM, between 0.3 nM and 0.5 nM, between 0.3 nM and 0.4 nM, or between 0.4 nM and 0.5 nM). In certain instances, the antibody binds to human IL-33 with a K_{D} of about 0.487 nM. In certain instances, the antibody binds to human IL-33 with a K_{D} of about 0.338 nM. Any of the preceding K_{D} values may be determined by surface plasmon resonance (SPR) (see, e.g., Examples 1 and 2).

In some embodiments, wherein the antibody binds to IL-33 bound to soluble interleukin 1 receptor like 1 (sST2) protein and to IL-33 not bound to sST2 with within two-fold (e.g., within one-and-a-half-fold or within one-fold) K_{D}. In some embodiments, the antibody binds to IL-33 bound to sST2 protein with substantially the same K_{D} as to IL-33 not bound to sST2. In some embodiments, the sST2 is human sST2 (hsST2). In some instances, the antibody binds to IL-33 bound to sST2 (e.g., hsST2) protein with a K_{D}≤1 nM. In some instances, the antibody binds to IL-33 bound to sST2 (e.g., hsST2) protein with a K_{D} between 0.01 nM and 1 nM (e.g., between 0.01 and 0.9 nM, between 0.01 and 0.8 nM, between 0.01 and 0.7 nM, between 0.01 and 0.6 nM, between 0.01 and 0.5 nM, between 0.05 and 0.9 nM, between 0.1 and 0.9 nM, between 0.2 and 0.9 nM, between 0.3 and 0.9 nM, between 0.2 and 0.6 nM, between 0.3 and 0.5 nM, between 0.3 and 0.4 nM, or between 0.4 and 0.5 nM; e.g., about 0.05 nM, about 0.1 nM, about 0.2 nM, about 0.3 nM, about 0.325 nM, about 0.34 nM, about 0.35 nM, about 0.4 nM, about 0.45 nM, about 0.475 nM, about 0.49 nM, about 0.5 nM, about 0.6 nM, about 0.7 nM, about 0.8 nM, about 0.9 nM, or about 1 nM). In some instances, the antibody binds to IL-33 bound to sST2 (e.g., hsST2) protein with a K_{D}≤0.5 nM. In certain instances, the antibody binds to IL-33 bound to sST2 (e.g., hsST2) protein with a K_{D} between 0.1 nM and 0.5 nM (e.g., between 0.2 and 0.5 nM, between 0.3 nM and 0.5 nM, between 0.3 nM and 0.4 nM, or between 0.4 nM and 0.5 nM). In certain instances, the antibody binds to IL-33 bound to sST2 (e.g., hsST2) protein with a K_{D} of about 0.487 nM. In certain instances, the antibody binds to IL-33 bound to sST2 (e.g., hsST2) protein with a K_{D} of about 0.338 nM.

In some instances, any of the anti-IL-33 antibodies described herein (e.g., described above or below) may have one or more (e.g., 1, 2, or 3) of the following features: (i) the antibody specifically binds human IL-33 with a K_{D} of between about 0.1 nM and 0.5 nM; (ii) the antibody specifically binds reduced IL-33 with a K_{D} of between about 0.1 nM and 0.5 nM; and/or (iii) the antibody specifically binds to IL-33 bound to sST2 (e.g., hsST2) protein with a K_{D} of between about 0.1 nM and 0.5 nM. In some instances, any of the anti-IL-33 antibodies described herein may have one of the preceding features. In some instances, any of the anti-IL-33 antibodies described herein may have two of the preceding features. In some instances, any of the anti-IL-33 antibodies described herein may have all three of the preceding features.

Any of the anti-IL-33 antibodies provided herein may be a monoclonal antibody, including a chimeric, humanized, or human antibody. In some instances, the anti-IL-33 antibody is a monoclonal antibody. In some instances, the anti-IL-33 antibody is a chimeric antibody. In some instances, the anti-IL-33 antibody is a humanized antibody. In some instances, the anti-IL-33 antibody is a rat antibody. In some instances, the anti-IL-33 antibody is an IgG antibody. In some instances, the antibody is an IgG1 antibody. In some instances, the antibody is a human IgG1 antibody. In some instances, the antibody is an IgG2a antibody. In some instances, the antibody is a rat IgG2a antibody.

Any of the anti-IL-33 antibodies provided herein may be an antibody fragment, e.g., a Fv, Fab, Fab′, scFv, diabody, or F(ab′)₂ fragment. In particular instances, the antibody fragment is an Fab fragment. In some instances, the antibody fragment specifically binds IL-33 (e.g., hIL-33; e.g., reduced IL-33; e.g., IL-33 bound to ST2).

In other instances, any of the anti-IL-33 antibodies provided herein may be a full-length antibody, e.g., an intact IgG1 antibody, an intact IgG4 antibody, or other antibody class or isotype as defined herein. In some instances, the antibody is an IgG4 antibody that comprises a mutation in the hinge region. In some instances, the mutation is a substitution mutation. In some instances, the substitution mutation is at amino acid residue S228 (EU numbering). In some instances, the substitution mutation is an S228P mutation.

Any of the anti-IL-33 antibodies provided herein may be a monospecific antibody. In other instances, any of the anti-IL-33 antibodies provided herein may be a multispecific antibody (e.g., a bispecific antibody).

In another aspect, provided herein is an antibody that binds to the same epitope of IL-33 as any one of the preceding antibodies.

In another aspect, provided herein is an antibody that competes for binding to IL-33 (e.g., human IL-33) with, or cross-blocks or is cross-blocked by any one of the preceding antibodies.

In a further aspect, any of the anti-IL-33 antibodies disclosed herein may incorporate any of the features, singly or in combination, as described in Sections 1-7 below:

### 1. Antibody Affinity

In certain embodiments, an antibody provided herein has a dissociation constant (K_{D}) of ≤1 μM, ≤100 nM, ≤10 nM, ≤1 nM, ≤0.1 nM, ≤0.01 nM, ≤1 pM, or ≤0.1 pM (e.g., 10⁻⁶ M or less, e.g., from 10⁻⁶ M to 10⁻⁹ M or less, e.g., from 10⁻⁹ M to 10⁻¹³ M or less). In certain instances, the antibody binds to IL-33 with a K_{D}≤1 nM. In some instances, the antibody binds to IL-33 with a K_{D} between 0.01 nM and 1 nM (e.g., between 0.01 and 0.9 nM, between 0.01 and 0.9 nM, between 0.01 and 0.8 nM, between 0.01 and 0.7 nM, between 0.01 and 0.6 nM, between 0.01 and 0.5 nM, between 0.05 and 0.9 nM, between 0.1 and 0.9 nM, between 0.2 and 0.9 nM, between 0.3 and 0.9 nM, between 0.2 and 0.6 nM, between 0.3 and 0.5 nM, between 0.3 and 0.4 nM, or between 0.4 and 0.5 nM; e.g., about 0.05 nM, about 0.1 nM, about 0.2 nM, about 0.3 nM, about 0.325 nM, about 0.34 nM, about 0.35 nM, about 0.4 nM, about 0.45 nM, about 0.475 nM, about 0.49 nM, about 0.5 nM, about 0.6 nM, about 0.7 nM, about 0.8 nM, about 0.9 nM, or about 1 nM). In some instances, the antibody binds to IL-33 with a K_{D}≤0.5 nM. In certain instances, the antibody binds to IL-33 with a K_{D} between 0.1 nM and 0.5 nM (e.g., between 0.2 and 0.5 nM, between 0.3 nM and 0.5 nM, between 0.3 nM and 0.4 nM, or between 0.4 nM and 0.5 nM). In certain instances, the antibody binds to IL-33 with a K_{D} of about 0.487 nM. In certain instances, the antibody binds to IL-33 with a K_{D} of about 0.338 nM.

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al. J. Mol. Biol. 293:865-881, 1999). To establish conditions for the assay, MICROTITER® multi-well plates (Thermo Scientific) are coated overnight with 5 μg/mL of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (NUNC™ #269620), 100 pM or 26 PM [¹²⁵I]-antigen are mixed with serial dilutions of a Fab of interest (e.g., consistent with assessment of the anti-VEGF antibody, Fab-12, in Presta et al. Cancer Res. 57:4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN®-20) in PBS. When the plates have dried, 150 μL/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. In a particular embodiment, a Series S Protein A sensor chip (Cytiva, Marlborough, MA, USA) was used to capture recombinant chimeric human IgG versions of anti-hIL-33 Abs using a BIACORE® 8K instrument (Cytiva, Marlborough, MA, USA). Antibody binding to human histidine-tagged hIL-33 (Genentech, South San Francisco, CA, USA) was measured using multi-cycle kinetics.

Sensorgrams were recorded using an injection time of 2 minutes with a flow rate of 30 μL/min, at 25° C. or 37° C., and with a running buffer of 10 mM N-2-hydroxyethylpiperazine-N′-2-ethanesulfonic acid (HEPES), pH 7.4, 150 mM sodium chloride (NaCl), 3 mM ethylenediaminetetraacetic acid (EDTA), and 0.005% TWEEN® 20. After injection, dissociation of hIL-33 from the antibody was monitored for 10 minutes in running buffer. The surface was regenerated between binding cycles with a 30 μL injection of 10 mM Glycine hydrochloric acid (HCI) pH 1.5. After subtraction of a blank containing running buffer only, association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ See, for example, Chen et al. (J. Mol. Biol. 293:865-881, 1999).

### 2. Antibody Fragments

Any of the antibodies provided herein may be an antibody fragment (e.g., an antigen-binding (e.g., IL-33-binding) antibody fragment). Antibody fragments include, but are not limited to, Fab, Fab′, Fab′-SH, F(ab′)2, Fv, and scFv fragments, and other fragments described below. In certain instances, the antibody fragment is an Fab. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134, 2003; and Hollinger et al. Proc. Natl. Acad. Sci. USA 90:6444-6448, 1993. Triabodies and tetrabodies are also described in Hudson et al. Nat. Med. 9:129-134, 2003.

Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al. Proc. Natl. Acad. Sci. USA, 81:6851-6855, 1984). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:1619-1633, 2008, and are further described, e.g., in Riechmann et al. Nature 332:323-329, 1988; Queen et al. Proc. Natl. Acad. Sci. USA 86:10029-10033, 1989; U.S. Pat. Nos. 5,821,337, 7,527,791, 6,982,321, and 7,087,409; Kashmiri et al. Methods 36:25-34, 2005 (describing specificity determining region (SDR) grafting); Padlan, Mol. Immunol. 28:489-498, 1991 (describing “resurfacing”); Dall'Acqua et al. Methods 36:43-60, 2005 (describing “FR shuffling”); and Osbourn et al. Methods 36:61-68, 2005 and Klimka et al. Br. J. Cancer, 83:252-260, 2000 (describing the “guided selection” approach to FR shuffling). Human framework regions that may be used for humanization include but are not limited to: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al. Proc. Natl. Acad. Sci. USA, 89:4285, 1992; and Presta et al. J. Immunol., 151:2623, 1993); human mature (somatically mutated) framework regions or human germline framework regions (see, e.g., Almagro et al. Front. Biosci. 13:1619-1633, 2008); and framework regions derived from screening FR libraries (see, e.g., Baca et al. J. Biol. Chem. 272:10678-10684, 1997 and Rosok et al. J. Biol. Chem. 271:22611-22618, 1996).

### 4. Human Antibodies

In certain embodiments, an antibody provided herein is a human antibody. Human antibodies can be produced using various techniques known in the art. Human antibodies are described generally in van Dijk et al. Curr. Opin. Pharmacol. 5:368-74, 2001 and Lonberg, Curr. Opin. Immunol. 20:450-459, 2008.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125, 2005. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE™ technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001, 1984; Brodeur et al. Monoclonal Antibody Production Techniques and Applications, pp. 51-63 (Marcel Dekker, Inc., New York, 1987); and Boerner et al. J. Immunol. 147:86, 1991). Human antibodies generated via human B-cell hybridoma technology are also described in Li et al. Proc. Natl. Acad. Sci. USA, 103:3557-3562, 2006. Additional methods include those described, for example, in U.S. Pat. No. 7,189,826 (describing production of monoclonal human IgM antibodies from hybridoma cell lines) and Ni, Xiandai Mianyixue, 26 (4): 265-268, 2006 (describing human-human hybridomas). Human hybridoma technology (Trioma technology) is also described in Vollmers et al. Histology and Histopathology 20 (3): 927-937, 2005 and Vollmers et al. Methods and Findings in Experimental and Clinical Pharmacology 27 (3): 185-91, 2005.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies disclosed herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al. Nature 348:552-554, 1990; Clackson et al. Nature 352:624-628, 1991; Marks et al. J. Mol. Biol. 222:581-597, 1992; Marks et al. in Methods in Molecular Biology 248:161-175 (Lo, ed., Human Press, Totowa, NJ, 2003); Sidhu et al. J. Mol. Biol. 338 (2): 299-310, 2004; Lee et al. J. Mol. Biol. 340 (5): 1073-1093, 2004; Fellouse, Proc. Natl. Acad. Sci. USA 101 (34): 12467-12472, 2004; and Lee et al. J. Immunol. Methods 284 (1-2): 119-132, 2004.

In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol., 12:433-455, 1994. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. EMBO J. 12:725-734, 1993. Finally, naive libraries can also be made synthetically by cloning unrearranged V-gene segments from stem cells, and using PCR primers containing random sequence to encode the highly variable HVR3 regions and to accomplish rearrangement in vitro, as described by Hoogenboom et al. J. Mol. Biol., 227:381-388, 1992. Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, for example, a bispecific antibody. Multispecific antibodies are monoclonal antibodies that have binding specificities for at least two different sites. In certain embodiments, bispecific antibodies may bind to two different epitopes of IL-33. In certain embodiments, one of the binding specificities is for IL-33 and the other is for any other antigen. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein et al. Nature 305:537, 1983; WO 93/08829; and Traunecker et al. EMBO J. 10:3655, 1991), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al. Science, 229:81, 1985); using leucine zippers to produce bispecific antibodies (see, e.g., Kostelny et al. J. Immunol., 148 (5): 1547-1553, 1992); using “diabody” technology for making bispecific antibody fragments (see, e.g., Hollinger et al. Proc. Natl. Acad. Sci. USA 90:6444-6448, 1993); and using single-chain Fv (scFv) dimers (see, e.g. Gruber et al. J. Immunol. 152:5368, 1994); and preparing trispecific antibodies as described, e.g., in Tutt et al. J. Immunol. 147:60, 1991.

Engineered antibodies with three or more functional antigen binding sites, including “Octopus antibodies,” are also included herein (see, e.g. US 2006/0025576A1).

The antibody or fragment herein also includes a “Dual Acting Fab” or “DAF” comprising an antigen binding site that binds to IL-33 as well as another, different antigen (see, US 2008/0069820, for example).

The use of knobs-into-holes as a method of producing multispecific antibodies is described, e.g., in U.S. Pat. No. 5,731, 168, WO2009/089004, US2009/0182127, US2011/0287009, Marvin and Zhu, Acta Pharmacol. Sin. (2005) 26 (6): 649-658, and Kontermann (2005) Acta Pharmacol. Sin., 26:1-9.

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis.

Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Exemplary amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more HVR (e.g., CDR) residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs (e.g., CDRs), e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196, 2008), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al. ed., Human Press, Totowa, NJ, 2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. HVR-H3 and HVR-L3 in particular are often targeted.

In certain embodiments, substitutions, insertions, or deletions may occur within one or more HVRs (e.g., CDRs) so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham et al. Science 244:1081-1085, 1989. In this method, a residue or group of target residues (e.g., charged residues such as Arg, Asp, His, Lys, and Glu) are identified and replaced by a neutral or negatively charged amino acid (e.g., Ala or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino-and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N-or C-terminus of the antibody to an enzyme (e.g., for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, for example, Wright et al. TIBTECH 15:26-32, 1997. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody disclosed herein may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. 2003/0157108 and 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. J. Mol. Biol. 336:1239-1249, 2004; Yamane-Ohnuki et al. Biotech. Bioeng. 87:614,2004. Examples of cell lines capable of producing defucosylated antibodies include Lec13 CHO cells deficient in protein fucosylation (Ripka et al. Arch. Biochem. Biophys. 249:533-545, 1986; US 2003/0157108; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng. 87:614, 2004; Kanda et al. Biotechnol. Bioeng. 94 (4): 680-688, 2006; and WO 2003/085107).

Antibodies variants are further provided with bisected oligosaccharides, e.g., in which a biantennary oligosaccharide attached to the Fc region of the antibody is bisected by GIcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch et al. Annu. Rev. Immunol. 9:457-492, 1991. Non-limiting examples of in vitro assays to assess ADCC activity of a molecule of interest is described in U.S. Pat. No. 5,500,362 (see, e.g., Hellstrom et al. Proc. Natl. Acad. Sci. USA 83:7059-7063, 1986 and Hellstrom et al. Proc. Natl. Acad. Sci. USA 82:1499-1502, 1985; U.S. Pat. No. 5,821,337 (see Bruggemann et al. J. Exp. Med. 166:1351-1361, 1987). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, for example, in an animal model such as that disclosed in Clynes et al. Proc. Natl. Acad. Sci. USA 95:652-656, 1998. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al. J. Immunol. Methods 202:163, 1996; Cragg et al. Blood 101:1045-1052, 2003; and Cragg et al. Blood 103:2738-2743, 2004). FcRn binding and in vivo clearance/half-life determinations can also be performed using methods known in the art (see, e.g., Petkova et al. Intl. Immunol. 18 (12): 1759-1769, 2006).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312; and Shields et al. J. Biol. Chem. 9 (2): 6591-6604, 2001).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), for example, as described in US Pat. No. 6,194,551, WO 99/51642, and Idusogie et al. J. Immunol. 164:4178-4184, 2000.

Antibodies with increased half-lives and improved binding to the neonatal Fc receptor (FcRn), which is responsible for the transfer of maternal IgGs to the fetus (Guyer et al. J. Immunol. 117:587, 1976 and Kim et al. J. Immunol. 24:249, 1994), are described in US2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan et al. Nature 322:738-40, 1988; U.S. Pat. Nos. 5,648,260 and 5,624,821; and WO 94/29351 concerning other examples of Fc region variants.

### d) Cysteine Engineered Antibody Variants

In certain embodiments, it may be desirable to create cysteine engineered antibodies, for example, “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly (n-vinyl pyrrolidone) polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al. Proc. Natl. Acad. Sci. USA 102:11600-11605, 2005). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Any of the antibodies (e.g., anti-IL-33 antibodies) described herein may be produced using recombinant methods and compositions, for example, as described in U.S. Pat. No. 4,816,567. In one embodiment, an isolated nucleic acid encoding an anti-IL-33 antibody described herein, or a set of nucleic acids together encoding the antibody, is provided. Such nucleic acid(s) may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid(s) are provided. In a further embodiment, a host cell comprising such nucleic acid(s) is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, for example, a rat cell, a Chinese Hamster Ovary (CHO) cell, 293 cell, or lymphoid cell (e.g., Y0, NS0, Sp20 cell). In one embodiment, a method of making an anti-IL-33 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-IL-33 antibody, a nucleic acid encoding an antibody, or a set of nucleic acids together encoding the antibody, for example, as described above, is isolated and inserted into one or more vectors for further cloning and/or expression in a host cell. Such nucleic acid(s) may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, for example, U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross Nat. Biotech. 22:1409-1414, 2004 and Li et al. Nat. Biotech. 24:210-215, 2006.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, for example, U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59, 1977); baby hamster kidney cells (BHK); mouse sertoli cells (TM4 cells as described, e.g., in Mather Biol. Reprod. 23:243-251, 1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68, 1982; MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216, 1980); and myeloma cell lines such as Y0, NS0 and Sp2/0. For a review of certain mammalian host cell lines suitable for antibody production, see, e.g., Yazaki et al. Methods in Molecular Biology, Vol. 248 (B.K.C. Lo, ed., Humana Press, Totowa, NJ), pp. 255-268, 2003.

### C. Assays

Anti-IL-33 antibodies provided herein may be identified, screened for, or characterized for their physical/chemical properties and/or biological activities by various assays known in the art.

### 1. Binding Assays and Other Assays

In one aspect, an anti-IL-33 antibody disclosed herein, an immunoconjugate comprising the anti-IL-33 antibody disclosed herein, or an immunoconjugate disclosed herein is tested for its antigen-binding activity, for example, by known methods such as ELISA, Western blot, and the like.

In an exemplary ELISA assay, a first immobilized anti-IL-33 antibody or immunoconjugate is incubated in a solution comprising IL-33 (e.g., reduced IL-33 or IL-33 bound to ST2). After incubation under conditions permissive for binding of the first antibody or immunoconjugate to IL-33 (e.g., reduced IL-33 or IL-33 bound to ST2), excess unbound IL-33 is removed. A solution containing a second, biotinylated, anti-hIL-33 antibody or immunoconjugate is added and incubated with the first immobilized anti-IL-33 antibody or immunoconjugate and bound IL-33 (e.g., reduced IL-33 or IL-33 bound to ST2). Bound reduced hIL-33 is then detected using a readout (e.g., with horseradish peroxidase-conjugated streptavidin (GE Healthcare Life Sciences, Pittsburgh, PA, USA) and its substrate 3,3′,5,5′-tetramethylbenzidine (Moss Inc., Pasadena, MD, USA)).

In another aspect, binding assays include iPCRs, which are assays in which the presence or level of a target molecule (e.g., a target biomolecule, e.g., a protein) is detected by an antibody-DNA conjugate. In some embodiments, an iPCR assay uses a first antibody conjugated to a biotin and a second antibody conjugated to a nucleic acid moiety, wherein the first and second antibodies bind to the same target antigen. Both the first and second antibodies are mixed with a solution containing the target antigen, and the resulting mixture is incubated together, e.g., within a PCR well. PCR amplification is then used to detect the presence of the nucleic acid moiety, and thereby indicate the detection of the target antigen. In some embodiments, the iPCR assay is used to detect IL-33 (e.g., human IL-33; e.g., reduced IL-33). In some embodiments, the first and second antibodies are anti-IL-33 antibodies. In some embodiments, the first and second antibodies share 90%, 95%, 96%, 97%, 98%, 99%, or 100% amino acid sequence identity. In some embodiments, the first and second antibodies do not share the same amino acid sequence (e.g., the amino acid sequences of the first and second antibodies share no more than 1%, 2%, 3%, 4%, 5%, 10%, 15%, or 20% amino acid sequence identity). Examples of iPCR assays are described in Sano T et. al., Science 1992, 258:120-122 and Niemeyer et al., Trends Microbiol. 2017, 8:65.

In another aspect, assays are provided for using anti-IL-33 antibodies or immunoconjugates, e.g., the anti-IL-33 antibodies or immunoconjugates disclosed herein, to detect the presence or level of IL-33 (e.g., human IL-33, reduced IL-33, oxidized IL-33, total IL-33, unbound IL-33, and IL-33 bound to ST2) in a biological sample e.g., in immunohistochemistry (IHC), immunofluorescence (IF), immunoblotting (e.g., Western blotting), flow cytometry (e.g., FACS™), or Enzyme-Linked Immunosorbant Assay (ELISA) assays. In certain embodiments, an antibody of the invention is tested for such activity.

In another aspect, competition assays may be used to identify an antibody that competes with an anti-IL-33 antibody disclosed herein for binding to IL-33. In certain embodiments, such a competing antibody binds to the same epitope (e.g., a linear or a conformational epitope) that is bound by an anti-IL-33 antibody disclosed herein. Detailed exemplary methods for mapping an epitope to which an antibody binds are provided in Morris “Epitope Mapping Protocols,” in Methods in Molecular Biology Vol. 66 (Humana Press, Totowa, NJ), 1996.

In an exemplary competition assay, immobilized IL-33 is incubated in a solution comprising a first labeled antibody that binds to IL-33 and a second unlabeled antibody that is being tested for its ability to compete with the first antibody for binding to IL-33. The second antibody may be present in a hybridoma supernatant. As a control, immobilized IL-33 is incubated in a solution comprising the first labeled antibody but not the second unlabeled antibody. After incubation under conditions permissive for binding of the first antibody to IL-33, excess unbound antibody is removed, and the amount of label associated with (e.g., bound to) immobilized IL-33 is measured. If the amount of label associated with (e.g., bound to) immobilized IL-33 is substantially reduced in the test sample relative to the control sample, then that indicates that the second antibody is competing with the first antibody for binding to IL-33. See Harlow et al. Antibodies: A Laboratory Manual Ch.14 (Cold Spring Harbor Laboratory, Cold Spring Harbor, NY), 1988.

In another embodiment, the competition of two antibodies for binding to the same epitope is determined by epitope binning. For example, labeled antigen is immobilized on a solid surface, and reacted with a saturating amount of a first antibody. A second competing antibody is then added. Any additional binding by the second antibody as detected by, e.g., OCTET® (ForteBio) or other bio-layer interferometry (BLI) techniques, indicates that the two antibodies bind to distinct, non-overlapping epitopes. No additional binding indicates that the two antibodies bind to the same or overlapping epitope. Several other methods, including ELISA, size exclusion chromatography, crystallography, HDX-MS, mutagenesis, and other SPR methods, can also be employed to demonstrate that two antibodies bind to the same or overlapping epitopes. Similar techniques can be used to determine whether an antibody cross-blocks, or is cross-blocked by, the antibody of the invention.

### 2. Activity Assays

In one aspect, the invention provides anti-IL-33 antibodies or immunoconjugates for detecting or measuring biological activity. Biological activity may include, for example, binding to IL-33 (e.g., IL-33 in the blood stream), or a peptide fragment thereof, either in vivo, in vitro, or ex vivo. In other embodiments, biological activity may include blocking or neutralizing IL-33, or preventing IL-33 from binding to a ligand, for example, a receptor (e.g., the IL-33 receptor ST2 and/or IL-1RAcP). In some embodiments, biological activity may include binding to site 1 on IL-33 and blocking of binding to the IL-33 receptor (i.e., ST2 and/or IL-1RAcP). Antibodies or immunoconjugates for detecting or measuring such biological activity in vivo and/or in vitro are also provided. In certain embodiments, an antibody disclosed herein is used for testing for such biological activity. In some embodiments, an anti-IL-33 antibody disclosed herein is used in testing inhibition in a cell-based IL-33 blocking assay. In some embodiments, an anti-IL-33 antibody disclosed herein is used for testing inhibition of IL-33-induced reporter activity in a cell-based blocking assay.

### D. Immunoconjugates

The invention also provides immunoconjugates comprising an anti-IL-33 antibody provided herein conjugated to one or more labels. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ¹³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, ß-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. In another embodiment, a label is a positron emitter. Positron emitters include but are not limited to ⁶⁸Ga, ¹⁸F, ⁶⁴Cu, ⁸⁶Y, ⁷⁶Br, ⁸⁹Zr, and ¹²⁴I. In a particular embodiment, a positron emitter is Zr. In some embodiments, the label is a nucleic acid, such as a single-stranded DNA.

In some embodiments, the immunoconjugate comprises an anti-IL-33 antibody provided herein conjugated to a nucleic acid, such as a DNA. In some embodiments, the immunoconjugate comprises an anti-IL-33 antibody conjugated to a 55-mer DNA. In some embodiments, the immunoconjugate comprises 15.21C7 antibody conjugated to a DNA having the sequence of SEQ ID NO: 38. In some embodiments, the immunoconjugate is purified by size exclusion column.

In some embodiments, the immunoconjugates comprises an anti-IL-33 antibody provided herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

### E. Methods and Compositions for Diagnostics and Detection

In certain embodiments, any one of the anti-IL-33 antibodies (e.g., 3F10 or 15.21C7) or immunoconjugates (e.g., comprising an anti-IL-33 antibody; e.g., comprising 3F10 or 15.21C7) provided herein is useful for detecting the presence of IL-33 (e.g., human IL-33, reduced IL-33, oxidized IL-33, total IL-33, unbound IL-33, and/or IL-33 bound to ST2) in a biological sample. The term “detecting” as used herein encompasses quantitative or qualitative detection.

In one instance, an anti-IL-33 antibody (e.g., 3F10 or 15.21C7) or immunoconjugate for use in a method of diagnosis or detection is provided. In another instance, the invention provides for the use of an anti-IL-33 antibody (e.g., 3F10 or 15.21C7) or immunoconjugate in the manufacture of a reagent for use in a method of diagnosis or detection.

In one instance, for example, a method of detecting the presence or level of IL-33 (e.g., human IL-33, reduced IL-33, oxidized IL-33, total IL-33, unbound IL-33, and/or IL-33 bound to ST2) in a biological sample, described below, is provided. In certain embodiments, the method comprises contacting the biological sample with an anti-IL-33 antibody (e.g., 3F10 or 15.21C7) or immunoconjugate as described herein under conditions permissive for binding of the anti-IL-33 antibody to IL-33 (e.g., human IL-33, reduced IL-33, oxidized IL-33, total IL-33, unbound IL-33, and/or IL-33 bound to ST2), and detecting whether a complex is formed between the anti-IL-33 antibody and IL-33 (e.g., human IL-33, reduced IL-33, oxidized IL-33, total IL-33, unbound IL-33, and/or IL-33 bound to ST2). Such method may be an in vitro or in vivo method. Anti-IL-33 antibodies of the invention (e.g., 3F10 and 15.21C7) or immunoconjugates of the invention can be used, for example, in immunoassays, including, for example, immunohistochemistry (IHC), immunofluorescence (IF), immunoblotting (e.g., Western blotting), flow cytometry (e.g., FACS™), Enzyme-linked Immunosorbant Assay (ELISA), and iPCR.

In certain embodiments, the method comprises contacting the biological sample with immunoconjugates as described herein in the presence of a second anti-IL-33 antibody (e.g., 1E1v8) that specifically binds an epitope on IL-33 that is different from the epitope to which the anti-IL-33 antibody in the immunoconjugate specifically binds, and detecting the presence of the bound immunoconjugate, under conditions permissive for binding of the anti-IL-33 antibody to IL-33 (e.g., human IL-33, reduced IL-33, oxidized IL-33, total IL-33, unbound IL-33, and/or IL-33 bound to ST2), and detecting whether a complex is formed between the anti-IL-33 antibody and IL-33 (e.g., human IL-33, reduced IL-33, oxidized IL-33, total IL-33, unbound IL-33, and/or IL-33 bound to ST2). In certain embodiments, the biological sample is treated with TCEP prior to said contacting. In some embodiments, the immunoconjugate is any one of the anti-IL-33 antibodies described herein (e.g., 15.21C7) conjugated to a nucleic acid (e.g., single-stranded DNA). In some embodiments, the immunoconjugate is purified by size-exclusion chromatography prior to said contacting. In some embodiments, said detecting is by iPCR.

The invention further provides for the use of an anti-IL-33 antibody in a method of diagnosing a subject suffering from a disorder (e.g., an IL-33-mediated disorder), the method comprising: determining the presence or level of IL-33 (e.g., human IL-33, reduced IL-33, oxidized IL-33, total IL-33, unbound IL-33, and/or IL-33 bound to ST2) in a sample obtained from the subject by contacting the sample with an anti-IL-33 antibody disclosed herein (e.g., 3F10 or 15.21C7) and detecting the presence of the bound antibody. The invention further provides for the use of an immunoconjugate in a method of diagnosing a subject suffering from a disorder (e.g., an IL-33-mediated disorder), the method comprising: determining the presence or level of IL-33 (e.g., human IL-33, reduced IL-33, oxidized IL-33, total IL-33, unbound IL-33, and/or IL-33 bound to ST2) in a sample obtained from the subject by contacting the sample with an immunoconjugate (e.g., comprising an anti-IL-33 antibody; e.g., comprising 3F 10 or 15.21C7) disclosed herein and detecting the presence of the bound antibody.

The invention yet further provides for the use of an anti-IL-33 antibody in the manufacture of a reagent for use in a method of diagnosing a subject suffering from a disorder (e.g., an IL-33-mediated disorder), the method comprising: determining the presence or level of IL-33 in a sample obtained from the subject by contacting the sample with an anti-IL-33 antibody disclosed herein (e.g., 3F10 or 15.21C7) and detecting the presence of the bound antibody. The invention yet further provides for the use of an immunoconjugate in the manufacture of a reagent for use in a method of diagnosing a subject suffering from a disorder (e.g., an IL-33-mediated disorder), the method comprising: determining the presence or level of IL-33 in a sample obtained from the subject by contacting the sample with an immunoconjugate (e.g., comprising an anti-IL-33 antibody; e.g., comprising 3F10 or 15.21C7) described herein and detecting the presence of the bound antibody.

Any suitable sample may be used. In some embodiments, the sample is a biological sample. In some embodiments, the biological sample is a blood sample, an ocular sample, a nasal sample, or a cell culture sample. In some embodiments, the blood sample is a plasma or serum sample. In some embodiments, the ocular sample is a vitreous humor or aqueous humor sample. In some embodiments, the ocular sample is a tissue sample. In some embodiments, the nasal sample is a nasosorption fluid sample.

In one aspect, an anti-IL-33 antibody or immunoconjugate disclosed herein may be used to select subjects eligible for therapy with an IL-33 axis binding antagonist (e.g., an anti-IL-33 antibody or an anti-ST2 antibody), for example, where IL-33 is a biomarker for selection of patients.

For example, the invention provides a method for selecting a therapy for a subject suffering from a disorder (e.g., an IL-33-mediated disorder), the method including: determining the presence or level (e.g., human IL-33, reduced IL-33, oxidized IL-33, total IL-33, unbound IL-33, and/or IL-33 bound to ST2) of IL-33 in a sample obtained from the subject by contacting the sample with an anti-IL-33 antibody disclosed herein (e.g., 3F10 or 15.21C7) and detecting the presence of the bound antibody; and selecting an IL-33 axis binding antagonist (e.g., an anti-IL-33 antibody or an anti-ST2 antibody) for the subject based on the presence or level of IL-33 (e.g., human IL-33, reduced IL-33, oxidized IL-33, total IL-33, unbound IL-33, and/or IL-33 bound to ST2) in the sample. In another example, the invention provides a method for selecting a therapy for a subject suffering from a disorder (e.g., an IL-33-mediated disorder), the method including: determining the presence or level (e.g., human IL-33, reduced IL-33, oxidized IL-33, total IL-33, unbound IL-33, and/or IL-33 bound to ST2) of IL-33 in a sample obtained from the subject by contacting the sample with an immunoconjugate (e.g., comprising an anti-IL-33 antibody; e.g., comprising 3F10 or 15.21C7) disclosed herein and detecting the presence of the bound antibody; and selecting an IL-33 axis binding antagonist (e.g., an anti-IL-33 antibody or an anti-ST2 antibody) for the subject based on the presence or level of IL-33 (e.g., human IL-33, reduced IL-33, oxidized IL-33, total IL-33, unbound IL-33, and/or IL-33 bound to ST2) in the sample.

In any of the aspects described herein, an IL-33 axis binding antagonist includes IL-33 binding antagonists, ST2 binding antagonists, and IL1RAP binding antagonists. Exemplary IL-33 axis binding antagonists include anti-IL-33 antibodies and antigen-binding fragments thereof (e.g., anti-IL-33 antibodies such as ANB-020 (AnaptysBio, Inc.) or any of the antibodies described in US2021-0284725A1,U.S. Pat. No. 10,093,730, EP1725261, U.S. Pat. No. 8,187,596, WO2011031600, WO2014164959, WO2015099175, WO2015106080, WO2016077381, or WO2021183849, which are each incorporated herein by reference in their entirety); polypeptides that bind IL-33 and/or its receptor (ST2 and/or IL-1RAcP) and block ligand-receptor interaction (e.g., ST2-Fc proteins, such as those described in WO 2014/152195, which is herein incorporated by reference in its entirety; immunoadhesins, peptibodies, and soluble ST2, or derivatives thereof); anti-IL-33 receptor antibodies (e.g., anti-ST2 antibodies, for example, astegolimab (also known as MSTT1041A), AMG-282 (Amgen) or STLM15 (Janssen) or any of the anti-ST2 antibodies described in WO 2013/173761 and WO 2013/165894, which are each incorporated herein by reference in their entirety; or ST2-Fc proteins, such as those described in WO 2013/173761; WO 2013/165894; or WO 2014/152195, which are each incorporated herein by reference in their entirety); and IL-33 receptor antagonists, such as small molecule inhibitors, aptamers that bind IL-33, and nucleic acids that hybridize under stringent conditions to IL-33 axis nucleic acid sequences (e.g., short interfering RNAs (siRNA) or clustered regularly interspaced short palindromic repeat RNAs (CRISPR-RNA or crRNA), including single guide RNAs (sgRNAs) having a crRNA and tracrRNA sequence as described in Mali et al. (Science. 339:823-26, 2013), which is incorporated herein by reference in its entirety).

In some embodiments, the sample is a biological sample. In some embodiments, the biological sample is a blood sample, an ocular sample, a nasal sample, or a cell culture sample. In some embodiments, the blood sample is a plasma or serum sample. In some embodiments, the ocular sample is a vitreous humor or aqueous humor sample. In some embodiments, the ocular sample is a tissue sample. In some embodiments, the nasal sample is a nasosorption fluid sample.

In any of the preceding methods, the biological sample may have a detectable level of IL-33 (e.g., human IL-33, reduced IL-33, oxidized IL-33, total IL-33, unbound IL-33, and/or IL-33 bound to ST2).

In some embodiments, the presence or level of IL-33 is the presence or level of total IL-33. In some embodiments, the presence or level of IL-33 is the presence or level of reduced IL-33. In some embodiments, the presence or level of IL-33 is the presence or level of oxidized IL-33. In some embodiments, the presence or level of IL-33 is the presence or level of human IL-33. In some embodiments, the presence or level of IL-33 is the presence or level of unbound IL-33. In some embodiments, the presence or level of IL-33 is the presence or level of IL-33 bound to sST2. In some embodiments, the sST2 is hsST2.

It is understood that any of the methods described above may include an immunoconjugate disclosed herein in place of or in addition to an anti-IL-33 antibody (e.g., any anti-IL-33 antibody disclosed herein).

### F. Biological Samples

In certain embodiments, any of the anti-IL-33 antibodies provided herein is useful for detecting the presence of IL-33 in a biological sample using methods known in the art or described herein. In some instances a biological sample includes a tissue or a cell sample. For example, a biological sample may include a cell or tissue from normal subjects or subjects having, predisposed to, or being tested for an IL-33 mediated disorder.

In certain instances the source of the tissue or cell sample may be solid tissue as from a fresh, frozen and/or preserved organ or tissue sample or biopsy or aspirate; blood or any blood constituents (e.g., whole blood, plasma, or serum); bodily fluids such as nasosorption fluid (NF), cerebral spinal fluid, amniotic fluid, peritoneal fluid, or interstitial fluid; cells from any time in gestation or development of the subject. Examples of tissue samples herein include, but are not limited to, ocular samples, including, but not limited to vitreous humor and aqueous humor samples. In some embodiments the biological sample is obtained from in vitro tissue or cell culture. Examples of biological samples herein include, but are not limited to, tumor biopsies, circulating tumor cells, serum or plasma, circulating plasma proteins, ascitic fluid, primary cell cultures or cell lines derived from tumors or exhibiting tumor-like properties, as well as preserved tumor samples, such as formalin-fixed, paraffin-embedded (FFPE) tumor samples or frozen tumor samples.

In some embodiments the biological sample contains compounds which are not naturally intermixed with the tissue in nature such as preservatives, anticoagulants, buffers, nutrients, antibiotics, or the like. In certain embodiments the biological sample has been exposed to and/or contains one or more fixatives. Fixatives that can be used with methods and compositions of the invention include formalin, glutaraldehyde, osmium tetraoxide, acetic acid, ethanol, acetone, picric acid, chloroform, potassium dichromate and mercuric chloride and/or stabilizing by microwave heating or freezing.

In some embodiments, the biological sample is from a subject having, predisposed to, or being tested for an IL-33 mediated disorder. In any of the preceding embodiments, the IL-33 mediated disorder may be an ocular disorder, an inflammatory condition, an immune disorder, a fibrotic disorder, or an eosinophilic disorder. For example, in some instances, an inflammatory condition may be asthma, airway hyperresponsiveness, airway inflammation, sepsis, septic shock, atopic dermatitis, allergic rhinitis, rheumatoid arthritis, or chronic obstructive pulmonary disease (COPD). In some instances, an immune disorder may be asthma, rheumatoid arthritis, allergy, atopic allergy, anaphylaxis, anaphylactic shock, allergic rhinitis, psoriasis, inflammatory bowel disease (IBD), Crohn's disease, diabetes, or liver disease. In some instances, the fibrotic disease may be idiopathic pulmonary fibrosis (IPF). In some instances, the eosinophilic disorder may be an eosinophil-associated gastrointestinal disorder (EGID). In some instances, the EGID may be eosinophilic esophagitis. In particular instances, the IL-33-mediated disorder may be asthma, allergic rhinitis, atopic dermatitis, COPD, eosinophilic esophagitis, or pulmonary fibrosis (e.g., IPF). For example, in some instances, the IL-33-mediated disorder is asthma. In other instances, the IL-33-mediated disorder is pulmonary fibrosis (e.g., IPF).

In some instances of any of the preceding embodiments, the IL-33-mediated disorder may be an ocular disorder (e.g., any ocular disorder disclosed herein). Non-limiting ocular disorders include, for example, AMD (e.g., wet AMD, dry AMD, intermediate AMD, advanced AMD, and geographic atrophy (GA)), macular degeneration, macular edema, DME (e.g., focal, non-center DME and diffuse, center-involved DME), retinopathy, diabetic retinopathy (DR) (e.g., proliferative DR (PDR), non-proliferative DR (NPDR), and high-altitude DR), other ischemia-related retinopathies, ROP, retinal vein occlusion (RVO) (e.g., central (CRVO) and branched (BRVO) forms), CNV (e.g., myopic CNV), corneal neovascularization, diseases associated with corneal neovascularization, retinal neovascularization, diseases associated with retinal/choroidal neovascularization, central serous retinopathy (CSR), pathologic myopia, von Hippel-Lindau disease, histoplasmosis of the eye, FEVR, Coats' disease, Norrie Disease, retinal abnormalities associated with osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, including but not limited to CMV retinitis, ocular melanoma, retinal blastoma, conjunctivitis (e.g., infectious conjunctivitis and non-infectious (e.g., allergic) conjunctivitis), Leber congenital amaurosis (also known as Leber's congenital amaurosis or LCA), uveitis (including infectious and non-infectious uveitis), choroiditis (e.g., multifocal choroiditis), ocular histoplasmosis, blepharitis, dry eye, traumatic eye injury, Sjögren's disease, and other ophthalmic diseases wherein the disease or disorder is associated with ocular neovascularization, vascular leakage, and/or retinal edema or retinal atrophy. Additional exemplary ocular disorders include retinoschisis (abnormal splitting of the retina neurosensory layers), diseases associated with rubeosis (neovascularization of the angle) and diseases caused by the abnormal proliferation of fibrovascular or fibrous tissue, including all forms of proliferative vitreoretinopathy.

Exemplary diseases associated with corneal neovascularization include, but are not limited to, epidemic keratoconjunctivitis, vitamin A deficiency, contact lens overwear, atopic keratitis, superior limbic keratitis, terygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegener's sarcoidosis, scleritis, Stevens-Johnson syndrome, periphigoid radial keratotomy, and corneal graph rejection.

Exemplary diseases associated with choroidal neovascularization and defects in the retina vasculature, including increased vascular leak, aneurisms and capillary drop-out include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, retina edema (including macular edema), Eales disease, Behcet's disease, infections causing retinitis or choroiditis (e.g., multifocal choroidits), presumed ocular histoplasmosis, Best's disease (vitelliform macular degeneration), myopia, optic pits, pars planitis, retinal detachment (e.g., chronic retinal detachment), hyperviscosity syndromes, toxoplasmosis, trauma, and post-laser complications.

Exemplary diseases associated with atrophy of retinal tissues (photoreceptors and the underlying RPE) include, but are not limited to, atrophic or nonexudative AMD (e.g., geographic atrophy or advanced dry AMD), macular atrophy (e.g., atrophy associated with neovascularization and/or geographic atrophy), diabetic retinopathy, Stargardt's disease, Sorsby Fundus Dystrophy, retinoschisis and retinitis pigmentosa.

In some embodiments, the ocular disorder includes AMD (including wet AMD, dry AMD, and GA), retinopathy (e.g., DR and ROP), PCV, diabetic macular edema, dry eye disease, Behcet's disease, allergic conjunctivitis, and retina detachment.

In other embodiments, the ocular disorder includes intermediate AMD, advanced AMD, glaucoma, uveitis (e.g., infectious and non-infectious uveitis), retinitis pigmentosa, Leber Congenital Amaurosis, Stargardt's disease, high-altitude diabetic retinopathy, traumatic eye injury, and conjunctivitis (e.g., infectious conjunctivitis and non-infectious conjunctivitis).

For example, provided herein are methods of detecting the presence or level of IL-33 in a subject having, predisposed to, or being tested for an ocular disorder. In one example, the invention provides a method of detecting the presence or level of IL-33 in a subject having, predisposed to, or being tested for an ocular disorder, in which the detecting occurs prior to administration of an IL-33 axis binding antagonist (e.g., an anti-IL-33 antibody or an anti-ST2 antibody disclosed herein) to the subject. In another example, the invention provides a method of detecting IL-33 in a subject having, predisposed to, or being tested for an ocular disorder in a subject in need thereof, wherein the subject has been administered a therapeutically effective amount of an IL-33 axis binding antagonist, for example, an anti-IL-33 antibody or an anti-ST2 antibody disclosed herein. In some instances, the ocular disorder may be selected from the group consisting of age-related macular degeneration (AMD), including wet AMD, dry AMD, intermediate AMD, advanced AMD, and geographic atrophy (GA)), retinopathy (e.g., diabetic retinopathy (DR), retinopathy of prematurity (ROP), and high-altitude DR), polypoidal choroidal vasculopathy (PCV), diabetic macular edema, dry eye disease, Behcet's disease, retina detachment, glaucoma, uveitis (e.g., infectious and non-infectious uveitis), retinitis pigmentosa, Leber Congenital Amaurosis (also known as Leber's congenital amaurosis), Stargardt's disease, traumatic eye injury, and conjunctivitis (e.g., infectious conjunctivitis, non-infectious conjunctivitis, and allergic conjunctivitis). In some instances, the ophthalmologic disorder includes AMD (including wet AMD, dry AMD, and GA), retinopathy (e.g., DR and ROP), PCV, diabetic macular edema, dry eye disease, Behcet's disease, allergic conjunctivitis, and retina detachment. In other instances, the ocular disorder includes intermediate AMD, advanced AMD, glaucoma, uveitis (e.g., infectious and non-infectious uveitis), retinitis pigmentosa, Leber Congenital Amaurosis, Stargardt's disease, high-altitude diabetic retinopathy, traumatic eye injury, and conjunctivitis (e.g., infectious conjunctivitis and non-infectious conjunctivitis).

### G. Pharmaceutical Formulations

Pharmaceutical formulations of an IL-33 axis binding antagonist (e.g., IL-33 binding antagonist or ST2 binding antagonist, e.g., anti-IL-33 antibody or anti-ST2 antibody) disclosed herein are prepared by mixing such IL-33 axis binding antagonist having the desired degree of purity with one or more optional pharmaceutically acceptable carriers (see, e.g., Remington's Pharmaceutical Sciences 16th edition, Osol, A. Ed., 1980), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed., 1980.

Sustained-release preparations may be prepared. Suitable examples of sustained-release preparations include semipermeable matrices of solid hydrophobic polymers containing the antibody, which matrices are in the form of shaped articles, e.g., films, or microcapsules.

For delivery to the eye (ophthalmic delivery), an antibody disclosed herein may be combined, for example, with ophthalmologically acceptable preservatives, co-solvents, surfactants, viscosity enhancers, penetration enhancers, buffers, sodium chloride, and/or water. Preservatives may be included, for example, to inhibit microbial contamination during use. Suitable preservatives include: edetate disodium, methyl paraben, propyl paraben, sorbic acid, phenylethyl alcohol, chlorobutanol, polyquaternium-1, or other agents known in the art. Such preservatives are typically employed at a level of from 0.001 to 1.0% w/v. In some instances, a pharmaceutical formulation disclosed herein does not include a preservative. In certain instances, compositions intended to be administered topically to the eye may be formulated as eye drops or eye ointments. In some instances, the total amount of antibody will be about 0.001 to 1.0% (w/w), for example, about 0.01 to about 1.0% (w/w), of such a formulation.

The formulations to be used for in vivo administration are generally sterile. Sterility may be readily accomplished, for example, by filtration through sterile filtration membranes.

An IL-33 axis binding antagonist can be formulated in a polymeric formulation.

### H. Kits and Articles of Manufacture

In another aspect, a kit or an article of manufacture containing materials useful for the detection of IL-33 using any of the antibodies (e.g., anti-IL-33 antibodies) or immunoconjugates described above is provided. The kit or article of manufacture may include an anti-IL-33 antibody (e.g., any anti-IL-33 antibody disclosed herein). The kit or article of manufacture may include an immunoconjugate (e.g., comprising an anti-IL-33 antibody; e.g., comprising 3F10 or 15.21C7) described herein. The kit or article of manufacture may include a container and a label or package insert on or associated with the container. Suitable containers include, for example, bottles, vials, syringes, IV solution bags, etc. The containers may be formed from a variety of materials such as glass or plastic. The container may hold a composition which is by itself or combined with another composition effective for assaying, detecting, and/or measuring the presence or level (e.g., expression level) of IL-33 in a sample (e.g., a biological sample). At least one active agent in the composition is an antibody disclosed herein. The label or package insert may indicate that the composition is used for diagnosing or detecting the condition of choice (e.g., an IL-33 mediated disorder). Moreover, the kit or article of manufacture may comprise (a) a first container with a composition contained therein, wherein the composition comprises an antibody or an immunoconjugate disclosed herein; and (b) a second container with a composition contained therein, wherein the composition comprises one or more reagents. The kit or article of manufacture in this embodiment disclosed herein may further comprise a package insert indicating that the compositions can be used to detect IL-33. Alternatively, or additionally, the kit or article of manufacture may further comprise a second (or third) container comprising an acceptable buffer, such as phosphate-buffered saline, Ringer's solution, and dextrose solution. It may further include other materials desirable from a commercial and user standpoint, including other buffers, diluents, filters, needles, and syringes.

For example, provided herein is a kit that includes (a) any of the antibodies disclosed herein; and (b) a package insert comprising instructions for use of the antibody to detect the presence or level of IL-33 in a biological sample. In another example, provided herein is a kit that includes (a) any of the immunoconjugates disclosed herein; and (b) a package insert comprising instructions for use of the immunoconjugate to detect the presence or level of IL-33 in a biological sample.

It is understood that any of the above articles of manufacture may include an immunoconjugate disclosed herein in place of or in addition to an anti-IL-33 antibody (e.g., any anti-IL-33 antibody disclosed herein).

### III. Exemplary Embodiments

The exemplary embodiments below are intended to be purely exemplary of the present application and should therefore not be considered to limit the invention in any way.

Among the embodiments provided herein are:
1. An isolated antibody that specifically binds IL-33, or an antigen-binding fragment thereof, wherein the antibody comprises a binding domain comprising a heavy chain variable (VH) domain and a light chain variable (VL) domain, the binding domain comprising the following six complementarity-determining regions (CDRs):
(a) a CDR-H1 comprising the amino acid sequence of SEQ ID NO: 1;
(b) a CDR-H2 comprising the amino acid sequence of SEQ ID NO: 2;
(c) a CDR-H3 comprising the amino acid sequence of SEQ ID NO: 3;
(d) a CDR-L1 comprising the amino acid sequence of SEQ ID NO: 4;
(e) a CDR-L2 comprising the amino acid sequence of SEQ ID NO: 5; and
(f) a CDR-L3 comprising the amino acid sequence of SEQ ID NO: 6.
2. The antibody of embodiment 1, wherein the VH domain comprises the following framework regions (FRs):
(a) an FR-H1 comprising the amino acid sequence of SEQ ID NO: 11;
(b) an FR-H2 comprising the amino acid sequence of SEQ ID NO: 12;
(c) an FR-H3 comprising the amino acid sequence of SEQ ID NO: 13; and
(d) an FR-H4 comprising the amino acid sequence of SEQ ID NO: 14.
3. The antibody of embodiment 1 or 2, wherein the VL domain comprises the following FRs:
(a) an FR-L1 comprising the amino acid sequence of SEQ ID NO: 15;
(b) an FR-L2 comprising the amino acid sequence of SEQ ID NO: 16;
(c) an FR-L3 comprising the amino acid sequence of SEQ ID NO: 17; and
(d) an FR-L4 comprising the amino acid sequence of SEQ ID NO: 18.
4. The antibody of any one of embodiments 1-3, comprising (a) a VH domain comprising an amino acid sequence having at least 95% sequence identity to the amino acid sequence of SEQ ID NO: 7; (b) a VL domain comprising an amino acid sequence having at least 95% sequence identity to the amino acid sequence of SEQ ID NO: 8; or (c) a VH domain as in (a) and a VL domain as in (b).
5. The antibody of any one of embodiments 1-4, wherein the VH domain comprises the amino acid sequence of SEQ ID NO: 7.
6. The antibody of any one of embodiments 1-5, wherein the VL domain comprises the amino acid sequence of SEQ ID NO: 8.
7. An isolated antibody that specifically binds IL-33, or an antigen-binding fragment thereof, wherein the antibody comprises a binding domain comprising (a) a VH domain comprising the amino acid sequence of SEQ ID NO: 7 and (b) a VL domain comprising the amino acid sequence of SEQ ID NO: 8.
8. The antibody of any one of embodiments 1-7, wherein the antibody comprises a heavy chain (HC) having at least 95% sequence identity to the amino acid sequence of SEQ ID NO: 9; (b) a light chain (LC) having at least 95% sequence identity to the amino acid sequence of SEQ ID NO: 10; or (c) an HC as in (a) and an LC as in (b).
9. The antibody of any one of embodiments 1-8, wherein the HC comprises the amino acid sequence of SEQ ID NO: 9.
10. The antibody of any one of embodiments 1-9, wherein the LC comprises the amino acid sequence of SEQ ID NO: 10.
11. An isolated antibody that specifically binds IL-33, or an antigen-binding fragment thereof, wherein the antibody comprises an HC comprising the amino acid sequence of SEQ ID NO: 9 and an LC comprising the amino acid sequence of SEQ ID NO: 10.
12. An isolated antibody that specifically binds IL-33, or an antigen-binding fragment thereof, wherein the antibody comprises a binding domain comprising the following six CDRs:
(a) a CDR-H1 comprising the amino acid sequence of SEQ ID NO: 19;
(b) a CDR-H2 comprising the amino acid sequence of SEQ ID NO: 20;
(c) a CDR-H3 comprising the amino acid sequence of SEQ ID NO: 21;
(d) a CDR-L1 comprising the amino acid sequence of SEQ ID NO: 22;
(e) a CDR-L2 comprising the amino acid sequence of SEQ ID NO: 23; and
(f) a CDR-L3 comprising the amino acid sequence of SEQ ID NO: 24.
13. The antibody of embodiment 12, wherein the VH domain comprises the following FRs:
(a) an FR-H1 comprising the amino acid sequence of SEQ ID NO: 29;
(b) an FR-H2 comprising the amino acid sequence of SEQ ID NO: 30;
(c) an FR-H3 comprising the amino acid sequence of SEQ ID NO: 31; and
(d) an FR-H4 comprising the amino acid sequence of SEQ ID NO: 32.
14. The antibody of embodiment 12 or 13, wherein the VL domain comprises the following FRs:
(a) an FR-L1 comprising the amino acid sequence of SEQ ID NO: 33;
(b) an FR-L2 comprising the amino acid sequence of SEQ ID NO: 34;
(c) an FR-L3 comprising the amino acid sequence of SEQ ID NO: 35; and
(d) an FR-L4 comprising the amino acid sequence of SEQ ID NO: 36.
15. The antibody of any one of embodiments 12-14, comprising (a) a VH domain having at least 95% sequence identity to the amino acid sequence of SEQ ID NO: 25; (b) a VL domain having at least 95% sequence identity to the amino acid sequence of SEQ ID NO: 26; or (c) a VH domain as in (a) and a VL domain as in (b).
16. The antibody of any one of embodiments 12-15, wherein the VH domain comprises the amino acid sequence of SEQ ID NO: 25.
17. The antibody of any one of embodiments 12-16, wherein the VL domain comprises the amino acid sequence of SEQ ID NO: 26.
18. An isolated antibody that specifically binds IL-33, or an antigen-binding fragment thereof, wherein the antibody comprises a binding domain comprising (a) a VH domain comprising the amino acid sequence of SEQ ID NO: 25 and (b) a VL domain comprising the amino acid sequence of SEQ ID NO: 26.
19. The antibody of any one of embodiments 12-18, wherein the antibody comprises an HC having at least 95% sequence identity to the amino acid sequence of SEQ ID NO: 27; (b) an LC having at least 95% sequence identity to the amino acid sequence of SEQ ID NO: 28; or (c) an HC as in (a) and an LC as in (b).
20. The antibody of any one of embodiments 12-19, wherein the HC comprises the amino acid sequence of SEQ ID NO: 27.
21. The antibody of any one of embodiments 12-20, wherein the LC comprises the amino acid sequence of SEQ ID NO: 28.
22. An isolated antibody that specifically binds IL-33, or an antigen-binding fragment thereof, wherein the antibody comprises an HC comprising the amino acid sequence of SEQ ID NO: 27 and an LC comprising the amino acid sequence of SEQ ID NO: 28.
23. The antibody of any one of embodiments 1-22, wherein the antibody binds to oxidized or reduced IL-33.
24. The antibody of embodiment 23, wherein the antibody binds to oxidized IL-33 and to reduced IL-33 with within two-fold K_{D}.
25. The antibody of embodiment 24, wherein the antibody binds to oxidized IL-33 and to reduced IL-33 with substantially the same K_{D}.
26. The antibody of any one of embodiments 1-25, wherein the antibody binds to reduced IL-33.
27. The antibody of embodiment 26, wherein the antibody binds to reduced IL-33 with a K_{D}≤1 nM.
28. The antibody of embodiment 27, wherein the antibody binds to reduced IL-33 with a K_{D} between 0.01 nM and 1 nM.
29. The antibody of embodiment 27, wherein the antibody binds to reduced IL-33 with a Ko ≤ 0.5 nM.
30. The antibody of embodiment 29, wherein the antibody binds to reduced IL-33 with a K_{D} between 0.1 nM and 0.5 nM.
31. The antibody of embodiment 29 or 30, wherein the antibody binds to reduced IL-33 with a K_{D} of about 0.487 nM.
32. The antibody of embodiment 29 or 30, wherein the antibody binds to reduced IL-33 with a K_{D} of about 0.338 nM.
33. The antibody of any one of embodiments 1-32, wherein the antibody binds to human IL-33.
34. The antibody of any one of embodiments 1-33, wherein the antibody binds to IL-33 bound to soluble interleukin 1 receptor like 1 (sST2) protein and to IL-33 not bound to sST2 with within two-fold K_{D}.
35. The antibody of any one of embodiments 1-34, wherein the antibody binds to IL-33 bound to sST2 protein with substantially the same K_{D} as to IL-33 not bound to sST2.
36. The antibody of embodiment 34 or 35, wherein the sST2 is human sST2 (hsST2).
37. The antibody of any one of embodiments 1-36, wherein the antibody is a monoclonal antibody.
38. The antibody of any one of embodiments 1-18 and 23-37, wherein the antibody is a chimeric antibody.
39. The antibody of any one of embodiments 12-38, wherein the antibody is a rat antibody.
40. The antibody of any one of embodiments 1-36, wherein the antibody is an IgG antibody.
41. The antibody of any one of embodiments 1-18, 21, and 23-37, wherein the antibody is an IgG1 antibody.
42. The antibody of any one of embodiments 1-7, 10, and 12-20, wherein the antibody is an IgG2a antibody.
43. The antibody of any one of embodiments 1-36, wherein the antibody is an antibody fragment that specifically binds IL-33.
44. The antibody of embodiment 43, wherein the antibody fragment is selected from the group consisting of Fab, single chain variable fragment (scFv), Fv, Fab′, Fab′-SH, F(ab′)₂, and diabody.
45. An isolated nucleic acid encoding the antibody of any one of embodiments 1-44 or a set of isolated nucleic acids together encoding the antibody.
46. A vector or a set of vectors comprising the isolated nucleic acid or set of isolated nucleic acids of embodiment 45.
47. A host cell comprising the vector or the set of vectors of embodiment 46.
48. The host cell of embodiment 47, wherein the host cell is a mammalian cell.
49. The host cell of embodiment 48, wherein the mammalian cell is a rat cell.
50. The host cell of embodiment 48, wherein the mammalian cell is a Chinese hamster ovary (CHO) cell.
51. The host cell of embodiment 47, wherein the host cell is a prokaryotic cell.
52. The host cell of embodiment 51, wherein the prokaryotic cell is E. coli.
53. A method of producing an antibody that specifically binds to IL-33, the method comprising culturing the host cell of any one of embodiments 47-52 in a culture medium.
54. The method of embodiment 53, wherein the method further comprises recovering the antibody from the host cell or the culture medium.
55. An immunoconjugate comprising the antibody of any one of embodiments 1-44.
56. The immunoconjugate of embodiment 55, wherein the antibody is linked to a label.
57. The antibody of any one of embodiments 1-44 or the immunoconjugate of embodiment 55 or 56 for use in detecting the presence or level of IL-33 in a biological sample.
58. The antibody for use or the immunoconjugate for use of embodiment 57, wherein the detecting is by immunohistochemistry (IHC), immunofluorescence (IF), flow cytometry, Enzyme-Linked Immunosorbant Assay (ELISA), immunoblotting, or immune-polymerase chain reaction (iPCR).
59. The antibody for use or the immunoconjugate for use of embodiment 58, wherein the detecting is by ELISA.
60. The antibody for use or the immunoconjugate for use of embodiment 58, wherein the detecting is by iPCR.
61. The antibody for use or the immunoconjugate for use of any one of embodiments 57-60, wherein the biological sample is a blood sample, an ocular sample, a nasal sample, or a cell culture sample.
62. The antibody for use or the immunoconjugate for use of embodiment 61, wherein the blood sample is a plasma or serum sample.
63. The antibody for use or the immunoconjugate for use of embodiment 61, wherein the ocular sample is a vitreous humor or aqueous humor sample.
64. The antibody for use or the immunoconjugate for use of embodiment 61, wherein the ocular sample is a tissue sample.
65. The antibody for use or the immunoconjugate for use of embodiment 61, wherein the nasal sample is a nasosorption fluid sample.
66. The antibody for use or the immunoconjugate for use of any one of embodiments 57-65, wherein biological sample is from a subject having, or at risk of, an IL-33-mediated disorder.
67. The antibody for use or the immunoconjugate for use of embodiment 66, wherein the IL-33-mediated disorder is an ocular disorder, an inflammatory condition, an immune disorder, a fibrotic disorder, an eosinophilic disorder, an infection, pain, a central nervous system disorder, or a solid tumor.
68. The antibody for use or the immunoconjugate for use of embodiment 67, wherein the ocular disorder is age-related macular degeneration (AMD), retinopathy of the eye, polypoidal choroidal vasculopathy (PCV), diabetic macular edema, dry eye disease, Behcet's disease, retina detachment, glaucoma, uveitis, retinitis pigmentosa, Leber Congenital Amaurosis, Stargardt's disease, traumatic eye injury, or conjunctivitis.
69. The antibody for use or the immunoconjugate for use of embodiment 68, wherein the AMD is geographic atrophy (GA), wet AMD, or dry AMD.
70. The antibody for use or the immunoconjugate for use of embodiment 68, wherein the AMD is GA.
71. The antibody for use or the immunoconjugate for use of embodiment 68, wherein the AMD is intermediate AMD or advanced AMD.
72. The antibody for use or the immunoconjugate for use of embodiment 68, wherein the retinopathy of the eye is diabetic retinopathy (DR) or retinopathy of prematurity (ROP).
73. The antibody for use or the immunoconjugate for use of embodiment 68, wherein the retinopathy of the eye is high-altitude DR.
74. The antibody for use or the immunoconjugate for use of embodiment 68, wherein the conjunctivitis is infectious conjunctivitis or non-infectious conjunctivitis.
75. The antibody for use or the immunoconjugate for use of embodiment 68, wherein the conjunctivitis is allergic conjunctivitis.
76. The antibody for use or the immunoconjugate for use of embodiment 67, wherein the inflammatory condition is asthma, sepsis, septic shock, atopic dermatitis, allergic rhinitis, rheumatoid arthritis, or chronic obstructive pulmonary disease (COPD).
77. The antibody for use or the immunoconjugate for use of embodiment 67, wherein the immune disorder is asthma, rheumatoid arthritis, allergy, anaphylaxis, anaphylactic shock, allergic rhinitis, psoriasis, inflammatory bowel disease (IBD), Crohn's disease, diabetes, or liver disease.
78. The antibody for use or the immunoconjugate for use of embodiment 67, wherein the fibrotic disease is idiopathic pulmonary fibrosis (IPF).
79. The antibody for use or the immunoconjugate for use of embodiment 67, wherein the eosinophilic disorder is an eosinophil-associated gastrointestinal disorder (EGID).
80. The antibody for use or the immunoconjugate for use of embodiment 79, wherein the EGID is eosinophilic esophagitis.
81. The antibody for use or the immunoconjugate for use of any one of embodiments 66-80, wherein the subject is human.
82. The antibody for use or the immunoconjugate for use of any one of embodiments 57-81, wherein the presence or level of IL-33 is the presence or level of total IL-33.
83. The antibody for use or the immunoconjugate for use of any one of embodiments 57-81, wherein the presence or level of IL-33 is the presence or level of reduced IL-33.
84. The antibody for use or the immunoconjugate for use of any one of embodiments 57-83, wherein the presence or level of IL-33 is the presence or level of human IL-33.
85. The antibody for use or the immunoconjugate for use of any one of embodiments 57-84, wherein the presence or level of IL-33 is the presence or level of unbound IL-33.
86. The antibody for use or the immunoconjugate for use of any one of embodiments 57-84, wherein the presence or level of IL-33 is the presence or level of IL-33 bound to sST2.
87. The antibody for use or the immunoconjugate for use of embodiment 86, wherein the sST2 is hsST2.
88. A method of detecting the presence or level of IL-33 in a biological sample comprising:
(i) contacting the biological sample with the antibody of any one of embodiments 1-44 and detecting the presence of the bound antibody; or
(ii) contacting the biological sample with the immunoconjugate of embodiment 55 or 56 and detecting the presence of the bound immunoconjugate.
89. The method of embodiment 88, wherein the detecting is by IHC, IF, flow cytometry, ELISA, immunoblotting, or iPCR.
90. The method of embodiment 89, wherein the detecting is by ELISA.
91. The method of embodiment 89, wherein the detecting is by iPCR.
92. The method of embodiment 91, wherein the method comprises contacting the biological sample with the immunoconjugate of embodiment 55 or 56 in the presence of a second anti-IL-33 antibody that specifically binds an epitope on IL-33 that is different from the epitope to which the anti-IL-33 antibody in the immunoconjugate specifically binds, and detecting the presence of the bound immunoconjugate.
93. The method of embodiment 92, wherein the second anti-IL-33 antibody is 1E1v8.
94. The method of embodiment 92 or 93, wherein the biological sample is treated with tris(2-carboxyethyl)phosphine (TCEP) prior to said contacting.
95. The method of any one of embodiments 88-94, wherein the biological sample is a blood sample, an ocular sample, a nasal sample, or a cell culture sample.
96. The method of embodiment 95, wherein the blood sample is a plasma or serum sample.
97. The method of embodiment 95, wherein the ocular sample is a vitreous humor or aqueous humor sample.
98. The method of embodiment 95, wherein the ocular sample is a tissue sample.
99. The method of embodiment 95, wherein the nasal sample is a nasosorption fluid sample.
100. The method of any one of embodiments 88-99, wherein biological sample is from a subject having, or at risk of, an IL-33-mediated disorder.
101. The method of embodiment 100, wherein the IL-33-mediated disorder is an ocular disorder, an inflammatory condition, an immune disorder, a fibrotic disorder, an eosinophilic disorder, an infection, pain, a central nervous system disorder, or a solid tumor.
102. The method of embodiment 101, wherein the ocular disorder is AMD, retinopathy of the eye, PCV, diabetic macular edema, dry eye disease, Behcet's disease, retina detachment, glaucoma, uveitis, retinitis pigmentosa, Leber Congenital Amaurosis, Stargardt's disease, traumatic eye injury, or conjunctivitis.
103. The method of embodiment 102, wherein the AMD is GA, wet AMD, or dry AMD.
104. The method of embodiment 102, wherein the AMD is GA.
105. The method of embodiment 102, wherein the AMD is intermediate AMD or advanced AMD.
106. The method of embodiment 102, wherein the retinopathy of the eye is DR or ROP.
107. The method of embodiment 102, wherein the retinopathy of the eye is high-altitude DR.
108. The method of embodiment 102, wherein the conjunctivitis is infectious conjunctivitis or non-infectious conjunctivitis.
109. The method of embodiment 102, wherein the conjunctivitis is allergic conjunctivitis.
110. The method of embodiment 101, wherein the inflammatory condition is asthma, sepsis, septic shock, atopic dermatitis, allergic rhinitis, rheumatoid arthritis, or COPD.
111. The method of embodiment 101, wherein the immune disorder is asthma, rheumatoid arthritis, allergy, anaphylaxis, anaphylactic shock, allergic rhinitis, psoriasis, IBD, Crohn's disease, diabetes, or liver disease.
112. The method of embodiment 101, wherein the fibrotic disease is IPF.
113. The method of embodiment 101, wherein the eosinophilic disorder is an EGID.
114. The method of embodiment 113, wherein the EGID is eosinophilic esophagitis.
115. The method of any one of embodiments 100-114, wherein the subject is human.
116. The method of any one of embodiments 88-115, wherein the presence or level of IL-33 is the presence or level of total IL-33.
117. The method of any one of embodiments 88-115, wherein the presence or level of IL-33 is the presence or level of reduced IL-33.
118. The method of any one of embodiments 88-117, wherein the presence or level of IL-33 is the presence or level of human IL-33.
119. The method of any one of embodiments 88-118, wherein the presence or level of IL-33 is the presence or level of unbound IL-33.
120. The method of any one of embodiments 88-118, wherein the presence or level of IL-33 is the presence or level of IL-33 bound to sST2.
121. The method of embodiment 120, wherein the sST2 is hsST2.
122. The method of any one of embodiments 88-121, wherein the method further comprises selecting a treatment comprising an IL-33 axis binding antagonist for the subject based on the presence or level of IL-33 in the biological sample.
123. The method of any one of embodiments 88-121, wherein the method further comprises administering a therapeutically effective amount of an IL-33 axis binding antagonist to the subject.
124. The method of embodiment 122 or 123, wherein the IL-33 axis binding antagonist is an IL-33 binding antagonist.
125. The method of embodiment 124, wherein the IL-33 binding antagonist is an anti-IL-33 antibody.
126. An assay for identifying a subject having an IL-33 mediated disorder who is a candidate for treatment comprising an IL-33 axis binding antagonist, wherein the assay comprises determining the presence or level of IL-33 in a biological sample obtained from the subject using the antibody of any one of embodiments 1-44 or the immunoconjugate of embodiment 55 or 56.
127. A kit comprising:
(a) the antibody of any one of embodiments 1-44; and
(b) a package insert comprising instructions for use of the antibody to detect the presence or level of IL-33 in a biological sample.
128. A kit comprising:
(a) the immunoconjugate of embodiment 55 or 56; and
(b) a package insert comprising instructions for use of the immunoconjugate to detect the presence or level of IL-33 in a biological sample.

### IV. EXAMPLES

The following are examples of methods and compositions of the invention. It is understood that various other embodiments may be practiced, given the general description provided above.

### Example 1: Materials and Methods

Measurement of Human sST2 Levels

hsST2 levels were determined using the Human ST2/hIL-33R QUANTIKINE® kit ELISA per the manufacturer's instructions (#DST200, R&D Systems, Minneapolis, MN, USA). The hsST2 standard range is 7.81 pg/mL to 1000 pg/mL with a sensitivity of 15.6 pg/mL in buffer and a lowest detectable concentration of 1563 pg/mL in aqueous humor (minimum required dilution (MRD) is 1:100), 625 pg/mL in vitreous humor (MRD is 1:40), 2500 pg/mL in serum (MRD is 1:160), and 1250 pg/mL in NF (MRD is 1:80).

Measurement of hIL-33 Levels in Vitreous Humor Using Commercial Kits

The following kits were used according to manufacturer's instructions: Human IL-33 QUANTIKINE® kit ELISA (#D3300B, R&D Systems, Minneapolis, MN, USA), LEGEND MAX™ hIL-33 kit ELISA (#435907, Biolegend, San Diego, CA, USA), Invitrogen IL-33 Human ELISA kit (#BMS2048, ThermoFisher Scientific, Carlsbad, CA, USA), U-PLEX® hIL-33 assay (#K151WFK, MSD, Rockville, MD, USA), BIO-PLEX PRO™ Human Th17 Cytokine 15-Plex LUMINEX® assay (#171AA001M, Bio-Rad, Hercules, CA, USA).

In addition, two ELISAs were developed using commercial antibodies (Abs). For the first assay, 2 μg/mL of mouse anti-hIL-33 diluted in 0.05 M sodium carbonate buffer pH 9.6 (#ALX-804-840 PF-C100, Enzo, Farmingdale, NY, USA) was used to coat MAXISORP® plates (384-well, Nunc, Thermo Fisher Scientific, Rochester, NY, USA) overnight at 4° C. After 3 washes in 0.05% TWEEN® 20 in PBS, PH 7.4, plates were blocked using 0.5% bovine serum albumin (BSA), 15 ppm PROCLIN® 300 in PBS, PH 7.4 for 1 hour (h) at room temperature (RT). After 3 washes, titrated hIL-33 was added to the plates and incubated for 2 h at RT. The plates were washed 6 times and biotinylated goat anti-hIL-33 (#BAF3625, R&D Systems, Minneapolis, MN, USA) diluted at 1:1000 was incubated for 1 h at RT. Bound Abs were detected using streptavidin poly-horseradish peroxidase (HRP) 80 (poly-HRP80) (#65R-5119, Fitzgerald, Acton, MA, USA) and its substrate 3,3′,5,5′-tetramethylbenzidine (Moss Inc., Pasadena, MD, USA). The reaction was stopped with 1 M phosphoric acid and absorbance was read at 450 nm.

For the second ELISA, a similar protocol was followed with the exceptions that 2 μg/mL of rabbit anti-hIL-33 (#500-P261, Peprotech, Minneapolis, MN, USA) was used for coating the plate and 50 ng/ml of biotinylated mouse anti-hIL-33 clone 6H617 (#NBP2-273338, Novus, Centennial, CO, USA) was used for detection in combination with streptavidin HRP (#RPN1231, GE Healthcare Life Sciences, Chicago, IL, USA).

### Sensitivity

The lower and upper limits of quantitation (LLOQ and ULOQ) were defined as the lowest and highest hIL-33 concentrations that can be accurately quantitated. These values were determined from standard curves run in quadruplicates in 6 independent assays. The CVs and relative error (RE) were calculated for each point of the standard curves and the ones with a CV and RE below 20% were included in the reportable range.

### Accuracy (Spike Recovery)

Reduced hIL-33 was spiked into serum, plasma, and NF samples from different individuals and into pooled vitreous humor and aqueous humor samples from 2 individuals (3 to 4 replicates) to target high (4.5 pg/mL), mid (1.5 pg/mL), and low (0.5 pg/mL) concentrations. The same high, mid, and low concentrations were spiked into assay buffer as controls (Spiked concentration). Endogenous levels of reduced hIL-33 were determined from unspiked matrices. Spike recovery was calculated using the following formula: % Recovery=[Measured concentration/Expected concentration (endogenous+spike)]×100.

### Dilutional Linearity

Serum, plasma and NF samples from spike recovery experiment were diluted in assay buffer at 1:2, 1:4, 1:10, and 1:20 concentrations, whereas vitreous humor and aqueous humor were diluted at 1:2, 1:4 and 1:8 (for vitreous humor only) due to limited sample volume availability. The percent recovery from measured versus expected corrected concentrations was calculated.

The MRD was defined as the minimum required dilution for which the reduced hIL-33 concentration was between 80-120% of expected sample recovery. The lower limit of quantitation (LLOQ) in matrices was calculated by multiplying the LLOQ obtained in buffer by the MRD.

hsST2 Tolerance Determination

hsST2 tolerance was determined by spiking various concentrations of hsST2 (0.016 ng/mL-40 ng/ml) into hIL-33 standard curves. The reported value is the highest concentration of hsST2 for which an acceptable recovery of hIL-33 concentration was obtained. An acceptable recovery was defined as 100%+20% of the corresponding hIL-33 concentration measured in the absence of hsST2.

hIL-33 Immunodepletion

Immunodepletion was performed by adding buffer, biotinylated anti-hIL-33 conjugated beads, or anti-gp120 conjugated beads (isotype control) to human vitreous humor samples for 1 hour at room temperature (RT). After incubation, the conjugated beads were removed from the matrix. These two steps were repeated and the amount of hIL-33 immunodepleted from the matrix was calculated. The immunodepletion was considered acceptable if the quantity of hIL-33 in the sample was reduced by 100%±20% SD compared to the control (non-immunodepleted) matrix sample value.

Reduced and Oxidized hIL-33 Production and Quality Control (QC)

A recombinant hIL-33 consisting of an N-terminal sequence MHHHHHHGENLYFQG (His-tag and TEV cleavage site; SEQ ID NO: 37) followed by the coding sequence for amino acids 112-270 (UniProtKB/Swiss-Prot accession number 095760) was expressed in Escherichia coli. The protein was purified over a Ni-Excel chromatography followed by a purification on the SUPERDEX® S75 size exclusion column pre-equilibrated with PBS (phosphate buffered saline). Mass spectrometry indicated that purified hIL-33 had the expected molecular weight and amino acid sequence. From an examination of sodium dodecyl sulfate polyacrylamide (SDS-PAGE) gels, the purity of the material was estimated to be greater than 95%. An aliquot of the purified hIL-33 was further diluted into PBS buffer to obtain a final concentration of 0.3 mg/mL. Similarly, another aliquot of the purified IL-33 was diluted into 60% Iscove's Modified Dulbecco's Medium (IMDM) (Thermo Fischer Scientific, Waltham, MA) to obtain the same final protein concentration. The diluted proteins were then incubated at 37° C. for approximately 18 hours. Both diluted proteins were then analyzed on SDS-PAGE gel and using mass spectrometry to determine the extent to disulfide bond formation. The oxidized hIL-33 diluted into the IMDM media was subsequently buffer exchanged into the final PBS buffer to remove the media components. Reduced hIL-33 in PBS was supplemented with 0.1 mM Tris (2-carboxyethyl) phosphine (TCEP) reducing agent.

### Antibody Production

Nine Sprague Dawley rats (Charles River, Hollister, CA, USA) were immunized with hIL-33 (Genentech) at 100 ug/animal for the first dose and 50 μg/animal for the rest of the boosts, each divided among sites: intraperitoneal, s.c. (subcutaneous) at base of tail, s.c. at nape of neck, and s.c. in both hocks. A Toll-like receptor cocktail including monophosphoryl-lipid A (MPL) (Sigma-Aldrich, St. Louis, MO, USA), Poly (I: C), R848, and cytosine phosphodiester bond guanine (CpG) (InvivoGen, San Diego, CA, USA) was used as adjuvant for the first dose. Antigen-specific hybridomas were generated, sorted and screened as described (Goldstein et al., Commun Biol, 2019, 2:304). Briefly, Immunoglobulin G (IgG)⁺ hIL-33⁺ hybridomas were single cell sorted using hIL-33 conjugated to phycoerythrin (PE) (Novus Biological, Littleton, CO, USA) into 96-well plates and cultured for 7 days. Supernatants were screened by high throughput (HTP) ELISA using robotic platforms and hybridomas showing binding to hIL-33 by ELISA were scaled-up in 1 mL cultures. Supernatants were harvested and purified using a protein G affinity chromatography resin (GammaBind Plus, GE Healthcare, Pittsburgh, PA, USA). Anti-hIL-33 Abs were submitted for sequencing and cloned out for recombinant production.

### Characterization of Anti-hIL-33 Abs by Array-Based SPR Imaging System

An array-based SPR imaging system (CFM/IBIS, Carterra, Dublin, CA, USA) was used to analyze binding kinetics and epitope bin a panel of anti-hIL-33 MAbs. Purified hybridoma Abs were diluted at 10 mg/mL in 10 mM sodium acetate buffer pH 4.5. Using amine coupling, Abs were directly immobilized onto a SPR sensor prism CMD 200M chip (XanTec Bioanalytics, Germany) using a Continuous Flow MICROSPOTTER™ (Carterra, Dublin, CA, USA) to create an array of Abs. Kinetics and binning experiments were carried at 25° C. in a running buffer composed of 10 mM HEPES, PH 7.4, 150 mM NaCl, 3 mM EDTA and 0.005% TWEEN® 20 (HBS-TE). The IBIS MX96 SPRi (Carterra, Dublin, CA, USA) was used to evaluate binding of hIL-33 to the immobilized Abs. For kinetics analysis, concentration series of reduced and oxidized hIL-33 starting at 300 nM was injected for 3 minutes (min) and allowed to disassociate for 10 min. The surface was regenerated between cycles with 10 mM Glycine pH 1.7. The binding data was processed using Scrubber (BioLogic Software). For Ab binning, reduced and oxidized hIL-33 was first injected for 4 min at 100 nM and was followed by a second 4 min injection of purified Ab at 10 mg/mL in an HBS-TE running buffer. The surface was regenerated between cycles with 10 mM Glycine pH 1.7. The binding data were processed using Epitope Binning software tool (Carterra, Dublin, CA, USA).

### Characterization of Anti-hIL-33 Abs by Surface Plasmon Resonance (SPR)

A Series S Protein A sensor chip (Cytiva, Marlborough, MA, USA) was used to capture recombinant chimeric human IgG versions of anti-hIL-33 Abs using a BIACORE® 8 k instrument (Cytiva, Marlborough, MA, USA). Antibody binding to human histidine-tagged hIL-33 (Genentech, South San Francisco, CA, USA) was measured using multi-cycle kinetics. Sensorgrams were recorded using an injection time of 2 minutes with a flow rate of 30 μL/min, at 25° C. or 37° C., and with a running buffer of 10 mM N-2-hydroxyethylpiperazine-N′-2-ethanesulfonic acid (HEPES), pH 7.4, 150 mM sodium chloride (NaCl), 3 mM ethylenediaminetetraacetic acid (EDTA), and 0.005% TWEEN® 20. After injection, dissociation of hIL-33 from the antibody was monitored for 10 minutes in running buffer. The surface was regenerated between binding cycles with a 30 μL injection of 10 mM Glycine hydrochloric acid (HCl) pH 1.5. After subtraction of a blank containing running buffer only, sensorgrams were analyzed using a 1:1 Langmuir binding model with software supplied by the manufacturer to calculate the kinetics and binding constants.

Reduced hIL-33 ELISA

MAXISORP® plates (384-well, Nunc, Thermo Fisher Scientific, Rochester, NY, USA) were coated with 1 μg/mL rat IgG2a anti-human hIL-33 15.21C7 Ab (Genentech, South San Francisco, CA, USA) in PBS, pH 7.4 overnight at 4° C. Plates were then washed with 0.05% TWEEN® 20 in PBS, pH 7.4, and blocked with 0.5% bovine serum albumin (BSA), 15 ppm PROCLIN™ 300 in PBS, PH 7.4 for 1 hour (h) at RT. Reduced hIL-33 standard was stored in 1 mM TCEP to maintain the protein in its reduced form and samples were diluted using a two-and-a-half step dilution in ice-cold assay buffer (0.5% BSA, 0.05% TWEEN® 20, 15 ppm P_{RO}C_{LIN}™, 0.25% CHAPS, 5 mM EDTA, and 0.35 N NaCl in PBS, PH 7.4) and incubated in the plate for 2 h at RT. After 6 washes, 200 ng/ml biotinylated rat/human chimeric anti-hIL-33 3F10 MAb (Genentech, South San Francisco, CA, USA) diluted in 0.5% BSA, 0.05% TWEEN® 20, 15 ppm PROCLIN™ in PBS, pH 7.4 was incubated for 1 h at RT. Bound reduced hIL-33 was detected using 1:10,000 dilution of horseradish peroxidase-conjugated streptavidin (GE Healthcare Life Sciences, Pittsburgh, PA, USA) and its substrate 3,3′,5,5′-tetramethylbenzidine (Moss Inc., Pasadena, MD, USA). The reaction was stopped with 1 M phosphoric acid and absorbance was read at 450 nm. The titration curves were fitted using an in-house, 4-parameter regression program.

Total hIL-33 ELISA

Total hIL-33 ELISA was carried out similarly using a 1:1 volume to volume ratio (vol/vol) mixture of 0.5 μg/mL rat IgG2a anti-hIL-33 15.21C7 (Genentech, South San Francisco, CA, USA) and 2 μg/mL goat anti-hIL-33 Abs (R&D Systems, Minneapolis, MN, USA) for coat and 1:1 (vol/vol) mixture of 100 ng/ml biotinylated goat anti-huST2 (R&D Systems, Minneapolis, MN, USA) and 50 ng/ml biotinylated goat anti-hIL-33 (R&D Systems, Minneapolis, MN, USA) Abs for detection. The standards consisted of a 1:1 (vol/vol) mixture of oxidized and reduced hIL-33 prepared in ice-cold assay buffer containing 20 ng/ml of hsST2.

### Antibody-DNA Conjugation

A 55-mer DNA (SEQ ID NO: 38) 5′-NH2-/5AmMC6/TGAAGGTCCTTGGCGATCATTTCGGCGGTGATCGGATGCATTTGGCTACGTCCCT-3′(Integrated DNA Technologies, Coralville, IA) was activated with N-succinimidyl-(N-maleimidomethyl) cyclohexane-1-carboxylate (Pierce, Rockford, IL, USA). Rat IgG2a anti-human IL-33 clone 15.21C7 (Genentech, South San Francisco, CA, USA) was activated with N-succinimidyl-S-acetylthioacetate (Pierce, Rockford, IL, USA) and conjugated to the activated deoxyribonucleic acid (DNA) following manufacturer's instructions. Free (e.g., unbound) DNA was removed using a SUPEROSE® 12 gel filtration column. Free (e.g., unbound) antibody was removed using a VIVAPURE® Q mini column (Sartorius Stedim Biotech GmbH, Goettingen, Germany).

384-Well iPCR Method to Detect Reduced hIL-33

0.2 μg/mL biotinylated rat/human chimeric anti-hIL-33 3F10 capture antibody, standards or samples, and 25 ng/ml DNA-labeled rat IgG2a anti-hIL-33 15.21C7 detection antibody were preincubated in sample buffer (0.5% BSA, 0.05% TWEEN® 20, 15 ppm PROCLIN™, 0.25% 3- ((3-cholamidopropyl) dimethylammonio)-1-propanesulfonate (CHAPS), 5 mM EDTA, 0.35 N NaCl and 250 μg/mL calf thymus DNA in PBS, pH 7.4) for 2 h at room temperature. 250 μg/mL calf thymus DNA (Sigma, St. Louis, MO, USA) was heated to 95° C. for 10 min and then kept on ice for at least 5 min prior to addition to the assay buffer. Reduced hIL-33 (Genentech, South San Francisco, CA, USA) in 1 mM TCEP was used as the standard and prepared on ice to decrease oxidation. Reduced hIL-33 concentrations in human vitreous humor, aqueous humor, serum, and NF samples were prepared on ice using sample buffer. A streptavidin-coated 384-well real-time PCR plate (Roche Applied Science-Custom Biotech, Penzberg, Germany) was blocked for 2 h with SUPERBLOCK™ buffer (ThermoFisher scientific, Waltham, MA, USA) and washed 3 times with iPCR wash buffer (0.5% BSA, 0.05% TWEEN® 20, 15 ppm PROCLIN™ in PBS, pH 7.4) using a 384-well plate washer (BioTek, Winooski, VT, USA). Antibody-antigen mixture was then added to the plate, incubated for 20 min prior to washing 13 times with iPCR wash buffer. A 60× PCR mixture containing primer (5′-TGAAGGTCCTTGGCGATCA (SEQ ID NO: 39) and 5′-AGGGACGTAGCCAAATGCAT (SEQ ID NO: 40)) and probe (5′-TTCGGCGGTGATCGp-3′ (SEQ ID NO: 41), labeled with a fluorescein dye and a quencher) was diluted to 1× in Universal TAQMAN® Master Mix II (Applied Biosystems, Foster City, CA, USA) and PCR grade water before being added to the plate. Real-time PCR was carried out using a VIIA™ 7 Real-Time PCR system (Applied Biosystems, Foster City, CA, USA). The thermal cycling conditions were 95° C. for 10 min followed by 40 cycles of 95° C. for 15 s and 60° C. for 1 min. The standard curve was generated using a 4-parameter regression curve-fitting program (Genentech, South San Francisco, CA, USA).

### Human Biological Samples

Vitreous humor and aqueous humor samples from control and acute AMD postmortem donors were purchased from Lions Eye Institute for Transplant and Research (Tampa, FL, USA) or BiolVT (Westbury, NY, USA). Note that the stage (early, intermediate, advanced) or the type of the disease (wet versus dry AMD) was not disclosed in the sample sheet provided by the vendors. Serum, NF, and plasma samples from control and “self-reported” asthmatic patients were acquired from Genentech Healthy Services (South San Francisco, CA, USA).

### Statistical Analysis

Statistical tests were conducted using either an unpaired two-tailed Student's t-test or a Mann-Whitney test. Error bars depict the standard deviation. A p-value <0.05 was considered statistically significant.

### Example 2: Development of an Ultra-Sensitive Human IL-33 Biomarker Assay for Age-Related Macular Degeneration and Asthma Drug Development

Over the past decade, human Interleukin 33 (hIL-33) has emerged as a key contributor to the pathogenesis of numerous inflammatory diseases. Despite the existence of several commercial hIL-33 assays spanning multiple platform technologies, their ability to provide accurate hIL-33 concentration measurements and to differentiate between active (reduced) and inactive (oxidized) hIL-33 in various matrices remains uncertain. This is especially true for lower sample volumes, matrices with low hIL-33 concentrations, and matrices with elevated levels of soluble Interleukin 1 Receptor-Like 1 (sST2), an inactive form of ST2 that competes with membrane bound ST2 for hIL-33 binding.

The performance of several commercially available hIL-33 detection assays was tested in various human matrices. It was found that most tested commercial assays lacked the sensitivity to accurately detect reduced hIL-33 at biologically relevant levels (sub-to-low pg/mL), especially in the presence of human sST2 (hsST2) (e.g., hIL-33 bound to hsST2), and/or lacked sufficient target specificity. To address this, a sensitive and specific enzyme-linked immunosorbent assay (ELISA) capable of detecting reduced and total hIL-33 levels even in the presence of high concentrations of sST2 (e.g., hIL-33 bound to hsST2) was developed and validated. By incorporating the immuno-polymerase chain reaction (iPCR) platform, the sensitivity of this assay was increased for the reduced form of hIL-33 by ˜52-fold. Using this hIL-33 iPCR assay, hIL-33 was detected in postmortem human vitreous humor samples from donors with age-related macular degeneration (AMD) and hIL-33 levels were found to be significantly increased when compared to control individuals. No statistically significant difference was observed in aqueous humor from AMD donors nor in plasma and nasosorption fluid (NF) from asthma patients compared to control individuals.

Unlike existing commercial hIL-33 assays, the presently described hIL-33 bioassays are highly sensitive and specific and can accurately quantify hIL-33 in various human clinical matrices, including those with high levels of hsST2. The present results demonstrate the utility of these assays in clinical trials targeting the hIL-33/hST2 pathway.

### Results

Commercial Assays Failed to Detect Reduced hIL-33 With High Sensitivity

To identify a sensitive assay capable of detecting single-digit pg/mL concentrations of reduced hIL-33 in biological matrices, various commercial assay reagents and platforms that were used in the literature to report hIL-33 concentrations (Table 3) were compared. A series of anti-hIL-33 antibodies was prescreened and only the ones yielding the highest sensitivity in buffer were selected for testing in the ELISA format. Four selection criteria were established to qualify as a suitable biomarker assay for human matrices: 1) the limit of quantitation of the assay in VH must be <4 pg/mL as hIL-33 is expected to be absent or present at low levels under non to moderate pathology conditions; 2) the assay must tolerate at least 10 ng/mL hsST2 as hsST2 mean concentrations in VH and serum samples are 2.05 and 19.8 ng/mL, respectively (FIG. 1); 3) the assay must be specific, which was characterized by the capacity to immunodeplete 100%±20% of hIL-33 from hIL-33-containing human vitreous humor samples when incubated with an anti-hIL-33 Ab; and 4) the maximum sample volume must be 50 μL, as some human ocular matrices are challenging to obtain and a high dilution would compromise the sensitivity of the assay.

None of the commercial kit/reagent assays tested met all four criteria (Table 3). The Invitrogen Human IL-33 ELISA kit, Rabbit (Rb) xhIL-33 (Pepro)/Biotin goat xhIL-33 (R&D), the U-PLEX Human IL-33 MSD kit and the Bio-Plex Pro Human T helper (Th17) cytokine 15-Plex kit had the lowest limit of detection in vitreous humor (Table 3). However, none of them could tolerate the mean hsST2 concentrations expected in human matrices (FIG. 1). The QUANTERIX SIMOA® platform failed to improve the limit of quantitation of the mu anti-hIL-33 (Enzo)/Biotin-mu anti-hIL-33 (Novus) pair in buffer and the sample volume required was not compatible with assaying ocular samples (Table 3).

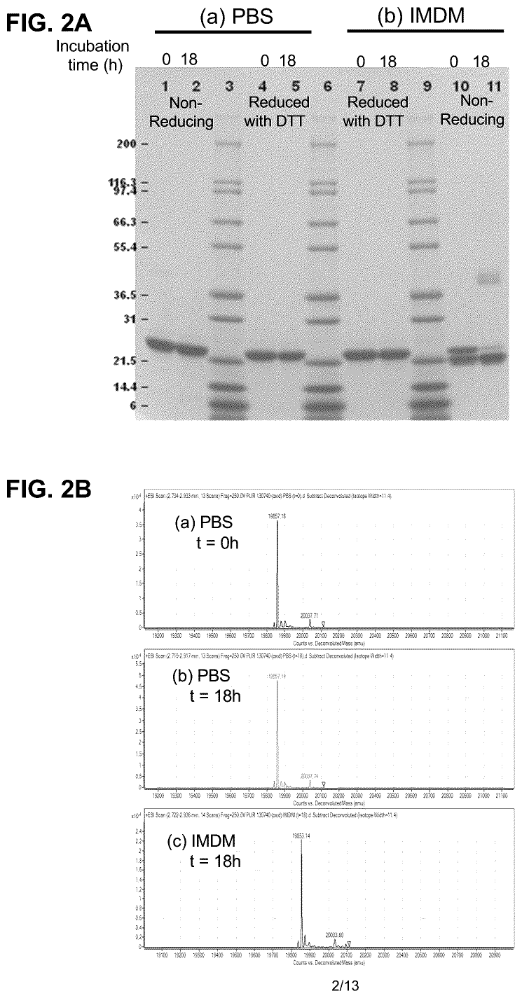

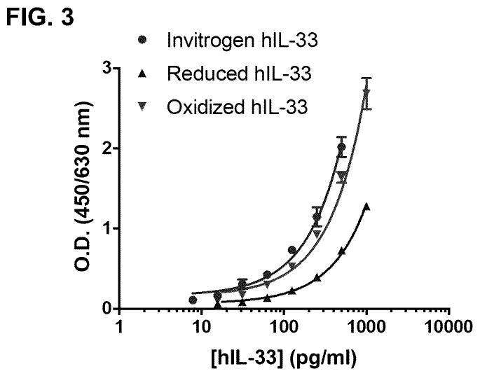

Aside from an unacceptable hsST2 tolerance, the Invitrogen Human IL-33 ELISA kit was sensitive and specific (Table 3) and met three out of four criteria. To investigate which forms of hIL-33 were specifically recognized in this kit, standard curves generated using hIL-33 provided in the kit or oxidized and reduced hIL-33 generated in house (FIG. 2) were compared. These results indicated that this ELISA kit was detecting mostly oxidized hIL-33 and has a low sensitivity towards reduced hIL-33 (FIG. 3). Altogether, this comparative study suggested that none of the tested commercial reagents/kits satisfied the criteria necessary to qualify as a sensitive reduced hIL-33 detection assay.

TABLE 3
Performance of commercial reagents and platforms to detect hIL-33

| | | Assay Criteria | | | | |
|---|---|---|---|---|---|---|
| | | | LLOQ in | | | |
| | | LLOQ in | vitreous | SST2 | | Sample |
| | | buffer | humor | Tolerance | Immunodepletion | Volume |
| Assay | Platform | (pg/mL) | (pg/mL) | (ng/ml) | (%) | (μL) |
| R&D Human IL-33 | ELISA | 6.25 | 62.5 | 1 | n/a | 50 |
| QUANTIKINE ® Kit | | | | | | |
| Biolegend LEGEND | | 15.6 | n/a | 10 | n/a | 50 |
| MAX ™ Human IL-33 Kit | | | | | | |
| Invitrogen | | 1.6 | 3.2 | 1 | 81 | 50 |
| IL-33 Human Kit | | | | | | |
| Rb xhIL-33 (Pepro)/ | | 0.8 | 8 | 0.25 | n/a | 25 |
| Biotin-goat xhIL-33 | | | | | | |
| (R&D) | | | | | | |
| Mu xhIL-33 (Enzo) / | | 20 | n/a | 0.5 | 59 | 25 |
| Biotin-mu xhIL-33 | | | | | | |
| (Novus) | | | | | | |
| U-PLEX ® Human IL-33 | ECL | 0.6 | 2.4 | 0.5 | 60 | 25 |
| MSD Kit | | | | | | |
| BIO-PLEX PRO ™ Human | LUMINEX ® | 1.3 | 2.6 | 0.016 | n/a | 50 |
| Th17 Cytokine 15-Plex | | | | | | |
| Kit | | | | | | |
| Mu xhL-33 (Enzo)/ | Quanterix | 20 | n/a | n/a | n/a | 150 |
| Biotin-mu xhIL-33 | | | | | | |
| (Novus) | | | | | | |

Parameters that did not meet the assay criteria selection are highlighted in bold; n/a represents parameters not tested.
LLOQ = lowest limit of quantitation.

Development of Reduced and Total hIL-33 ELISAs

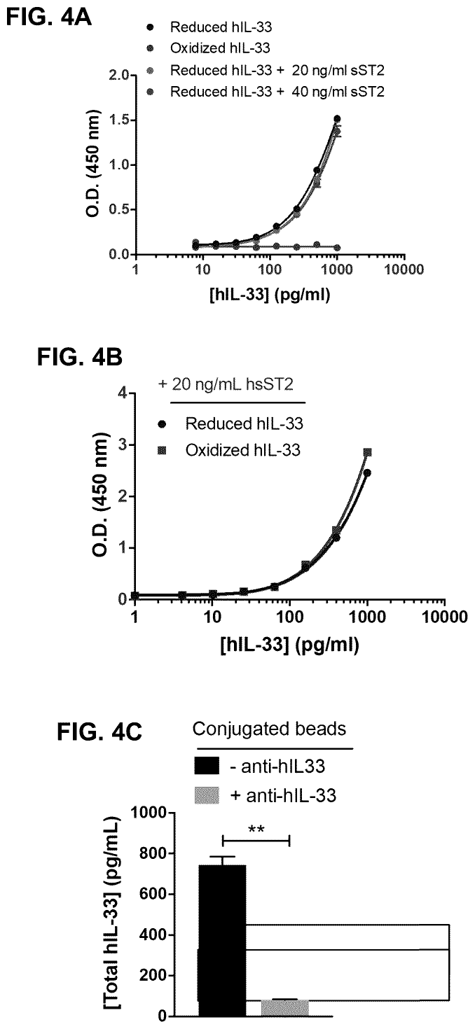

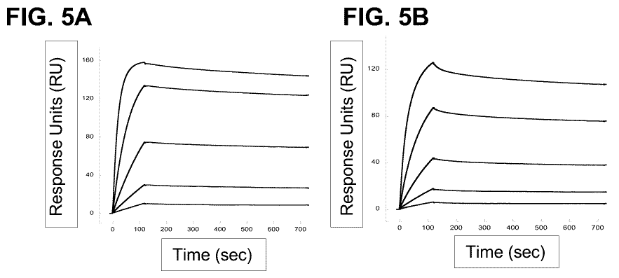

The successful development of two ELISAs capable of quantifying 1) reduced hIL-33 and 2) total hIL-33 (reduced and oxidized) in vitreous humor was accomplished through an extensive antibody generation and screening campaign followed by characterization and validation experiments. The rat immunization campaign resulted in the generation of 3026 antibodies. Array-based Surface Plasmon Resonance (SPR) was used to identify purified hybridoma Abs that specifically bound to reduced hIL-33with high affinity and represented different epitope groups that were non-competing with hsST2 through Ab binning. 88 combinations of the selected in-house monoclonal and commercial polyclonal Abs (MAbs or PAbs) pairs were screened for their ability to bind total, oxidized, or reduced hIL-33 by ELISA. Out of the 88 antibody pairs, only two pairs detected reduced hIL-33 with single-digit pg/mL sensitivity in buffer and only one pair (antibody clones 3F10 and 15.21C7) was hsST2 tolerant (up to 40 ng/ml) (FIG. 4A). Clones 3F10 and 15.21C7 bound reduced hIL-33 with constant dissociation (K_{D}) values of 0.487 and 0.338 nM, respectively (FIG. 5 and Table 4). The lower limit of quantitation (LLOQ) and the upper limit of quantitation (ULOQ) of the reduced hIL-33 ELISA were 4.1 pg/mL and 1000 pg/mL, respectively with inter-assay coefficient of variations (CVs) of 9.1% and 1.6% (Table 4). The assay was performed in 384 well plate format with an assay volume of 25 μL.

TABLE 4
Kinetics constants for anti-hIL-33 Abs 15.21C7
and 3F10 binding to reduced hIL-33 at 25° C.

| | Kₐ (1/Ms) | K_{d} (1/s) | K_{D} (nM) |
|---|---|---|---|
| 15.21C7 | 5.01E+05 | 1.46E−04 | 0.291 |
| 3F10 | 3.12E+05 | 2.43E−04 | 0.777 |

A total hIL-33 ELISA was implemented, in which a combination of reduced (15.21C7) and total hIL-33 specific antibodies was used as a coat. Upon addition of a saturating concentration of hsST2, an antibody cocktail consisting of anti-hIL-33 and anti-ST2 monoclonal antibodies was used to detect the totality of reduced and oxidized hIL-33 present in the mixture. The ratio of each antibody was extensively optimized to ensure equal detection of reduced and oxidized hIL-33 (FIG. 4B). The LLOQ and ULOQ of the total ELISA was 4.1 pg/mL and 1000 pg/mL, respectively, with inter-assay CVs of 10.5% and 0.7% (Table 5).

TABLE 5
Standard curves performance of reduced and total hIL-33 ELISA

| | Nominal | Measured | | |
|---|---|---|---|---|
| | conc | mean conc | % | % |
| | (pg/mL) | (pg/mL) | Recovery | CV |
| Reduced | 1000 | 991.5 | 99.2 | 1.6 |
| hIL-33 | 400 | 408.2 | 102.1 | 2.8 |
| ELISA | 160 | 158.5 | 99.0 | 1.7 |
| | 64 | 63.6 | 99.4 | 1.5 |
| | 25.6 | 26.2 | 102.3 | 7.0 |
| | 10.2 | 11.0 | 107.6 | 5.6 |
| | 4.10 | 3.83 | 93.6 | 9.1 |
| Total | 1000 | 1007.4 | 100.7 | 0.7 |
| hIL-33 | 400 | 407.5 | 101.9 | 2.5 |
| ELISA | 160 | 159.6 | 99.7 | 2.4 |
| | 64 | 64.7 | 101.1 | 8.4 |
| | 25.6 | 27.6 | 107.9 | 8.4 |
| | 10.2 | 9.56 | 93.4 | 9.6 |
| | 4.10 | 4.54 | 110.8 | 10.5 |

n = 6 independent assay runs.
LLOQ = 4.1 pg/mL,
ULOQ = 1000 pg/mL

Immunodepletion experiments confirmed that the assay was specific for total hIL-33 in control vitreous humor samples with a reduction of 89.1%±0.31% signal in the presence of beads conjugated anti-hIL-33 antibodies (FIG. 4C). The minimum required dilution was also established to be 1:2 for serum, plasma, and NF by testing total hIL-33 linearity dilution (Table 6). As the LLOQ of the assay is 4.1 pg/mL, the lowest limit of detection is 8.2 pg/mL for serum, plasma, and NF.

To summarize, a reduced and total hIL-33 ELISA that can detect single-digit pg/mL hIL-33 in the presence of hsST2 (e.g., hIL-33 bound to hsST2) was successfully implemented and developed.

TABLE 6
Dilution linearity of total hIL-33 endogenously
expressed in human matrices

| Dilution Factor | Observed | Corrected | % |
|---|---|---|---|
| in Matrix | pg/mL | pg/mL | Recovery |
| 1:2 serum | 184.66 | 369.33 | 104.9 |
| 1:20 serum | 15.70 | 314.08 | 89.2 |
| 1:100 serum | 3.72 | 372.49 | 105.8 |
| 1:2 plasma | 110.77 | 221.54 | 80.5 |
| 1:20 plasma | 14.31 | 286.17 | 104.0 |
| 1:100 plasma | 3.18 | 317.84 | 115.5 |
| 1:2 NF | 78.33 | 156.66 | 84.3 |
| 1:4 NF | 44.85 | 179.41 | 96.6 |
| 1:10 NF | 19.10 | 191.01 | 102.8 |
| 1:20 NF | 10.81 | 216.17 | 116.3 |

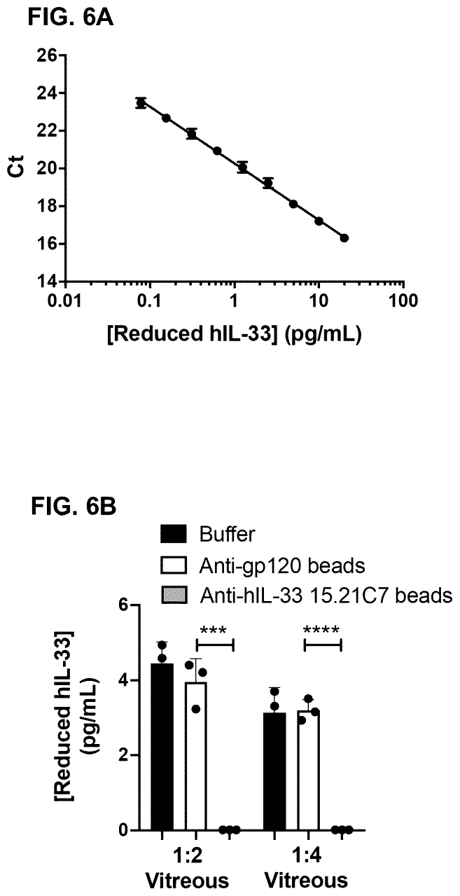

Development and Characterization of Reduced hIL-33 iPCR Assay Despite the increased sensitivity of the hIL-33 ELISA described above, it still failed to measure detectable levels of hIL-33 in a large proportion of human ocular samples and serum samples. Therefore, the iPCR platform that had previously showed great promise in increasing the sensitivity of detection assays was investigated. Similar to the reduced hIL-33 ELISA, the clones 3F10 and 15.21C7 were utilized, but were conjugated the latter to a 55-mer DNA that was amplified during the iPCR reaction, allowing for signal intensification. The iPCR platform dramatically increased sensitivity with a LLOQ of 0.078 pg/mL (±1.1%), representing a 52.6-fold improvement from the ELISA (Table 7) (FIG. 6A). Immunodepletion experiments confirmed that the assay was specific for reduced hIL-33 in control vitreous humor samples with a reduction of 99.6%±0.07% signal in the presence of beads conjugated to clone 15.21C7 Ab (FIG. 6B).

TABLE 7
Standard curve performance of reduced hIL-33 iPCR

| Nominal | Measured | | |
|---|---|---|---|
| conc | mean conc | % | % |
| (pg/mL) | (pg/mL) | Recovery | CV |
| 20 | 19.06 | 95.3 | 1.5 |
| 10 | 9.93 | 99.3 | 1.2 |
| 5 | 5.18 | 103.6 | 1.2 |
| 2.5 | 2.32 | 92.9 | 1.3 |
| 1.25 | 1.24 | 98.9 | 1.5 |
| 0.63 | 0.63 | 101.0 | 0.9 |
| 0.31 | 0.31 | 98.2 | 1.2 |
| 0.16 | 0.15 | 97.3 | 0.9 |
| 0.078 | 0.074 | 94.8 | 1.1 |

n = 6 independent assay runs.
LLOQ = 0.078 pg/mL,
ULOQ = 20 pg/mL

The assay precision was evaluated by measuring 3 concentrations (0.6, 1.8 and 6.5 pg/mL) of reduced hIL-33 diluted in assay buffer in quadruplicates for a total of 12 independent runs. All three controls had intra-and inter-assay CVs lower than 20% (Table 8), showing that the results generated were reproducible and precise.

TABLE 8
Precision of reduced hIL-33 iPCR assay

| | |
|---|---|
| Mean | % CV |

| Control | (pg/mL) | Inter-assay^{a} | Intra-assay^{b} |
|---|---|---|---|
| High Control | 6.52 | 15 | 9.4 |
| Mid Control | 1.82 | 6.9 | 13 |
| Low Control | 0.591 | 13 | 8.7 |

^{a}n = 12 runs;
^{b}n = 4 replicates

The assay linearity was then assessed using a set of pooled, individual control and diseased matrix samples in order to determine the minimum required dilution. In order to implement a biomarker assay that can be used across multiple indications, two ocular matrices, serum, plasma, and NF were tested. Due to low endogenous reduced hIL-33 concentrations (low pg/mL) and limited availability of human matrices, the range of dilutions tested was reduced. These results showed that the recoveries among dilution-adjusted concentrations of ocular, serum, plasma and NF matrices were within the ±20% acceptance criterion for linearity across all dilutions tested except for serum at 1:2 (% recovery=66.3%) (Table 9). Therefore, the minimum required dilution (MRD) for vitreous humor, aqueous humor, plasma and NF was 1:2 and 1:4 for serum. As the LLOQ of the assay is 0.078 pg/mL, the lowest concentrations that can be quantitated were 0.156 pg/mL for vitreous humor, aqueous humor, plasma, and NF, and 0.312 pg/mL for serum.

TABLE 9
Dilution linearity of reduced hIL-33 endogenously
expressed in human matrices

| Dilution Factor | Observed | Corrected | % |
|---|---|---|---|
| in Matrix | (pg/mL) | (pg/mL) | Recovery |
| 1:2 vitreous humor | 3.44 | 6.87 | 110.7 |
| 1:4 vitreous humor | 1.45 | 5.78 | 93.1 |
| 1:8 vitreous humor | 0.75 | 5.98 | 96.2 |
| 1:2 aqueous humor | 3.21 | 6.43 | 98.3 |
| 1:4 aqueous humor | 1.66 | 6.65 | 101.7 |
| 1:2 serum | 3.02 | 6.04 | 66.3 |
| 1:4 serum | 2.24 | 8.94 | 98.2 |
| 1:10 serum | 1.07 | 10.70 | 117.5 |
| 1:20 serum | 0.54 | 10.75 | 118.0 |
| 1:2 plasma | 4.77 | 9.55 | 96.4 |
| 1:4 plasma | 2.79 | 11.14 | 112.5 |
| 1:10 plasma | 1.03 | 10.27 | 103.7 |
| 1:20 plasma | 0.43 | 8.64 | 87.3 |
| 1:2 NF | 7.91 | 15.82 | 109.1 |
| 1:4 NF | 3.94 | 15.77 | 108.7 |
| 1:10 NF | 1.32 | 13.16 | 90.7 |
| 1:20 NF | 0.66 | 13.29 | 91.6 |

Additionally, spike recovery experiments were performed to evaluate accuracy. Low, mid, and high concentrations of reduced hIL-33 were added to these matrices diluted according to their respective MRD and determined the percent recovery as the ratio of measured concentration over expected concentration (endogenous +spike reduced hIL-33). All sample recovery was in the expected 80-120% range, demonstrating that the iPCR assay can accurately measure reduced hIL-33 in ocular, serum, plasma and NF matrices (Table 10).

TABLE 10
Accuracy test as measured by spike recovery
of reduced hIL-33 in matrices

| | vitreous | aqueous | | | |
|---|---|---|---|---|---|
| hIL-33 | humor | humor | Serum | Plasma | NF |
| (pg/mL) | (1:2) | (1:2) | (1:4) | (1:2) | (1:2) |
| Endogenous conc. | <0.078 | 0.59 | 2.24 | 5.73 | 7.91 |
| Spiked conc. (low) | 0.47 | 0.47 | 0.45 | 0.36 | 0.45 |
| Measured conc. | 0.41 | 1.14 | 3.14 | 7.26 | 9.78 |
| Expected conc. | 0.47 | 1.06 | 2.69 | 6.09 | 8.36 |
| (endo + spike) | | | | | |
| % Recovery | 86.5 | 107.1 | 117.0 | 119.2 | 117.0 |
| Endogenous conc. | <0.078 | 0.59 | 2.24 | 4.78 | 7.91 |
| Spiked conc. (mid) | 1.11 | 1.36 | 1.51 | 1.40 | 1.51 |
| Measured conc. | 0.93 | 1.73 | 3.48 | 5.90 | 11.0 |
| Expected conc. | 1.11 | 1.95 | 3.75 | 6.18 | 9.42 |
| (endo + spike) | | | | | |
| % Recovery | 84.1 | 88.7 | 92.8 | 95.5 | 116.8 |
| Endogenous conc. | <0.078 | 3.21 | 2.24 | 5.73 | 7.91 |
| Spiked conc. (high) | 4.25 | 4.25 | 4.48 | 5.22 | 4.48 |
| Measured conc. | 3.49 | 6.9 | 5.44 | 10.48 | 14.7 |
| Expected conc. | 4.25 | 7.47 | 6.72 | 10.95 | 12.39 |
| (endo + spike) | | | | | |
| % Recovery | 82.1 | 92.4 | 81.0 | 95.8 | 118.3 |

Reduced hIL-33 Levels Are Significantly Higher in Vitreous Humor Samples From Human Dry AMD as Compared to Control

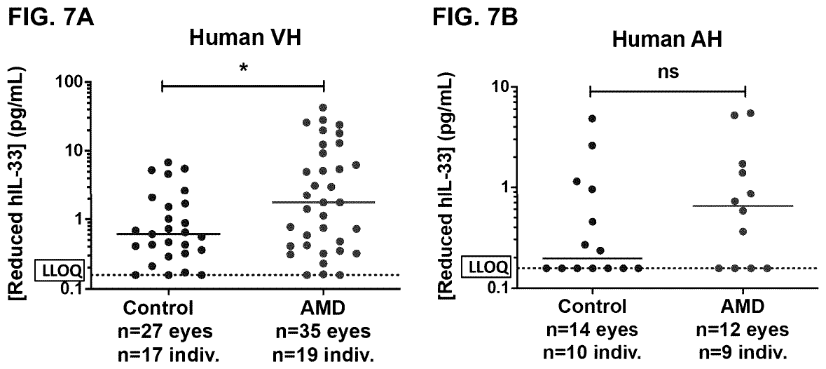

Using the iPCR assay, levels of reduced hIL-33 were measured in human vitreous humor and aqueous humor samples from postmortem normal and AMD donors. It should be noted that information regarding the severity (early versus advanced) or type (wet versus dry) of AMD from these donors was not available. A total of 88.7% of vitreous humor and 57.7% of aqueous humor samples were above LLOQ. A significant increase of reduced hIL-33 was observed in AMD versus control samples, consistent with increased presence of hIL-33 positive cells associated with GA (Xi et al., J Exp Med, 2016, 213:189-207) (FIG. 7A). An average of 3.3-fold higher amounts of reduced hIL-33 in postmortem AMD aqueous humor samples was also detected as compared to control aqueous humor samples, but the difference was not significant (P=0.2773) (FIG. 7B). Altogether, these results suggest that the reduced iPCR assay can be used as a predictive and pharmacodynamic biomarker assay.

Reduced hIL-33 is Not Significantly Upregulated in Plasma and NF From Self-Reported Asthma Patients

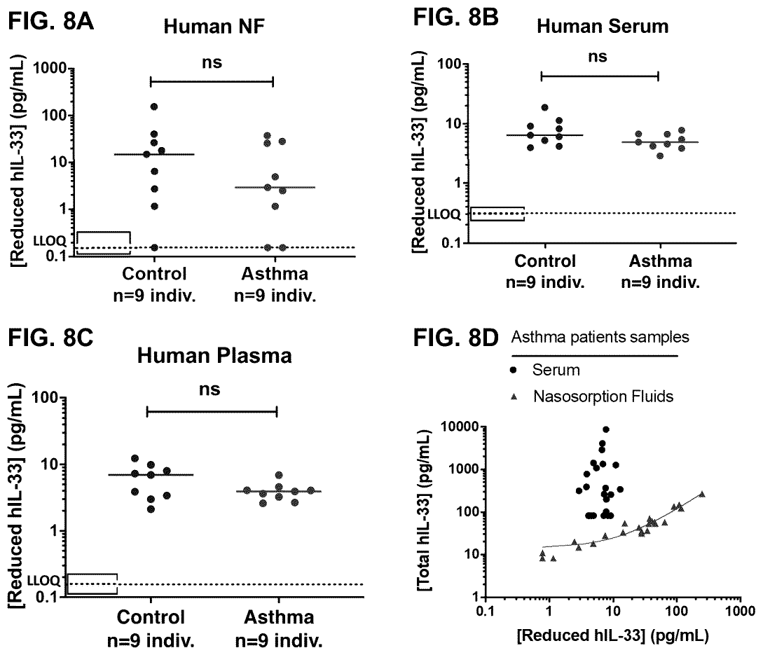

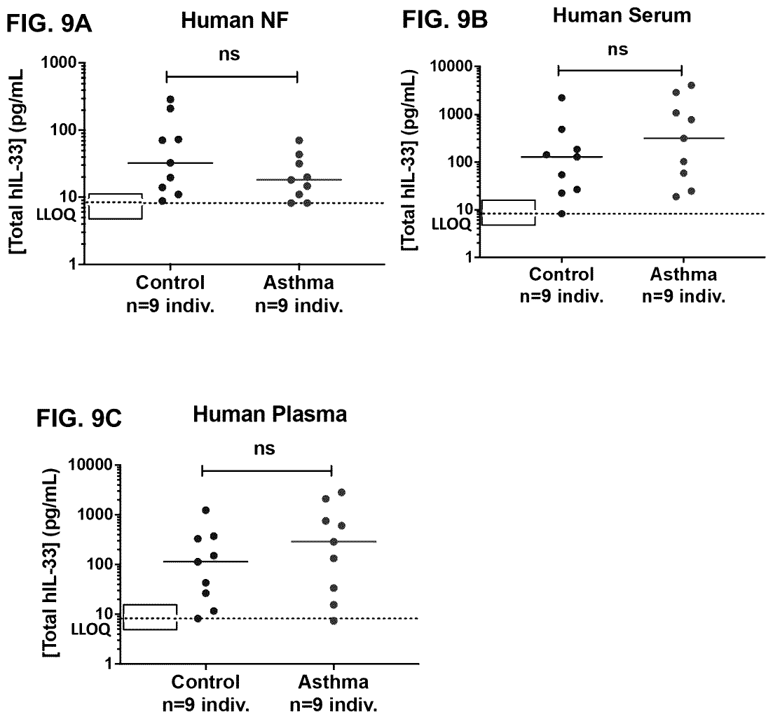

Several reports in the literature indicate that hIL-33 protein level is upregulated in asthma patients (Momen et al., Int J Prev Med, 2017, 8:65; Bahrami Mahneh et al., Int Arch Allergy Immunol, 2015, 168:193-196; Voloshyna et al., Clin Exp Allergy, 2015, 45:1554-1565; Raeiszadeh Jahromi et al., J Asthma, 2014, 51:1004-1013). However, the comprehensive evaluation of the commercial assays available to measure hIL-33 concentrations revealed that most if not all of them are unable to measure active reduced hIL-33 with the sensitivity required to make an informed interpretation. Here, using the reduced hIL-33 iPCR assay, levels of reduced hIL-33 in NF and serum from nine age-and gender-matched control and asthma patients were determined and compared. Note that both matrices were drawn from the same donors. These results indicated that there was no significant difference between reduced hIL-33 concentrations in NF and serum (FIG. 8A and 8B). In addition, the mean level of reduced hIL-33 in serum was 6.4 pg/mL (FIG. 8B). It is possible that this relatively high level could have been the result of the release of intracellular hIL-33 from the platelets during serum preparation. To rule out this hypothesis, similar measurements were performed in the matched plasma samples. The mean reduced hIL-33 concentration was similar to serum (6.9 pg/mL), confirming that the reduced hIL-33 concentration was not an artefact of samples preparation (FIG. 8C). The total hIL-33 concentrations (reduced and oxidized) in the same samples were also tested, and significant differences between normal and asthma samples were not observed (FIG. 9).

The average reduced hIL-33 concentration in human serum from asthma patients (n=23samples) was 6.7 pg/mL versus 1.06 ng/mL total hIL-33, demonstrating that most of the hIL-33 was oxidized and inactive (FIG. 8D). There was a significant correlation between reduced and total hIL-33 in NF samples, demonstrating that the majority of hIL-33 in this matrix is reduced and active (FIG. 8D). Altogether, these results do not support an upregulation of active hIL-33 in asthma patient serum and NF.

### Discussion

In this study, an extensive evaluation of commercially available assays to measure hIL-33 in biological matrices was provided. Similar to the observations made by Cohen et al. and Ketelaar et al., most, if not all, of the commercial assays tested did not meet the criteria for sensitive and specific measurement of active secreted hIL-33 (Cohen et al., Nat Commun, 2015, 6:8327; Ketelaar et al., Clin Exp Allergy, 2016, 46:884-887). This finding suggested that the bioactive hIL-33 levels in extracellular matrices reported in the literature should be interpreted with caution and supported the need to implement an assay to measure the availability of reduced hIL-33 in normal and diseased human patients. Reported here is the development of an ultra-sensitive iPCR assay compatible with a 384-well plate format that accurately measures reduced hIL-33 in ocular matrices, serum, plasma, and NF with a limit of detection of 0.156, 0.312, 0.156 and 0.156 pg/mL, respectively. The iPCR platform was chosen as iPCR has been successfully utilized to enhance detection sensitivity of antigens up to 2 logs as compared to standard ELISA. The 384-well plate format was especially attractive, as human ocular samples are extremely challenging to acquire, and typically less than 100 μL of aqueous humor sample can be obtained per donor. The fully characterized assay has the potential to be utilized as a predictive or pharmacodynamic biomarker assay.

First, levels of hIL-33 in human vitreous humor and aqueous humor samples from normal and AMD patients were measured. Contrary to rodent ocular samples that contain ng/ml of hIL-33 (data not shown), ˜1,000-fold less hIL-33 in human ocular samples was expected based on the work published by Takeuchi et al. (Takeuchi et al., PLoS One, 2015, 10: e0137358). In this study, the authors reported a mean of 17.1 pg/mL of hIL-33 in vitreous humor samples from patients with proliferative diabetic retinopathy with only 25% of the samples above the limit of detection. These results showed that the assay used in the study was suboptimal and could not tolerate ng/ml concentrations of hsST2 (FIG. 3). Using the iPCR assay, it was possible to detect hIL-33 in 88.7% of the vitreous humor samples tested (control and AMD samples). A significant increase in hIL-33 concentrations in postmortem AMD was observed compared to control vitreous humor samples, suggesting that hIL-33 may be upregulated in AMD, consistent with earlier findings. An increased level of hIL-33 in postmortem aqueous humor samples from donors with AMD was also measured compared to control donors, but the difference was not significant (P=0.2773). A higher number of aqueous humor samples may be required to establish whether a statistical significance can be reached. Overall, the hIL-33 concentrations obtained in vitreous humor samples were higher than in aqueous humor samples. This is in agreement with the predominant production of hIL-33 by cells in the posterior compartment of the eye, which is closer to the vitreous compartment than to the aqueous compartment (Xi et al., J Exp Med, 2016, 213:189-207).

Serum, plasma, and NF from normal versus asthma patients was also analyzed. A strong correlation between reduced and total hIL-33 was observed in the NF of asthmatic patients, indicating that most of the total hIL-33 is reduced, which may be explained by the proximity of a tissue expressing high levels of hIL-33.

In contrast, most of the hIL-33 in serum and plasma seems to be oxidized and therefore unable to bind hsST2. This observation confirms the prediction made by Cohen et al., who hypothesized that any free (e.g., unbound) hIL-33 reaching the plasma would likely be oxidized within a few hours (Cohen et al., Nat Commun, 2015, 6:8327). This regulatory mechanism is essential, as persistent elevation of IL-33 in serum has been shown to trigger lethal inflammation in a mouse model in which IL-33 nuclear localization signal was deleted (Bessa et al., J Autoimmun, 2014, 55:33-41).

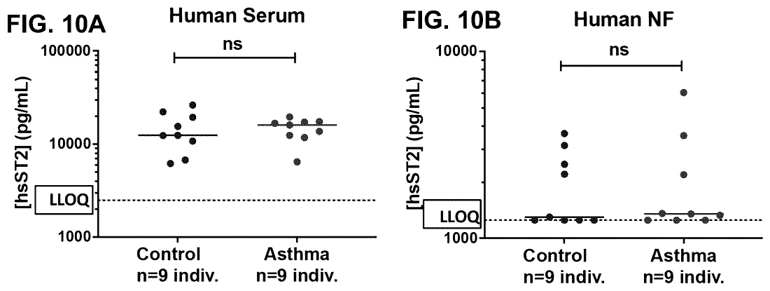

Importantly, the reduced hIL-33 iPCR assay is highly hsST2 tolerant, meaning that free (e.g., unbound) reduced hIL-33 and hsST2-bound hIL-33 cannot be distinguished. This implies that the 6.9 pg/mL (0.38 pM) of hIL-33 measured in plasma may not be fully active, as a fraction may be bound to its decoy receptor hsST2. The mean concentration of hsST2 in both control and asthma patients' samples groups was determined to be 14.7 ng/ml (0.4 nM) (FIG. 10A). Therefore it is likely that the majority or the entire reduced hIL-33 was in complex with hsST2 and unable to signal through its receptor. A similar assumption could not be made for the NF samples as hsST2 levels were below the LLOQ in about 60% of the samples (FIG. 10B).

This is the first study accurately reporting reduced and total hIL-33 in human serum and plasma. Other groups reported levels of serum hIL-33 across asthma patients with variable disease severity (Momen et al., Int J Prev Med, 2017, 8:65; Bahrami Mahneh et al., Int Arch Allergy Immunol, 2015, 168:193-196; Voloshyna et al., Clin Exp Allergy, 2015, 45:1554-1565; Raeiszadeh Jahromi et al., J Asthma, 2014, 51:1004-1013). All used commercial kits to generate hIL-33 levels that, according to the thorough characterization, were not suitable to accurately detect reduced hIL-33 for the following reasons: 1) these assays could not tolerate the hsST2 level present in serum (˜20 ng/ml), 2) their limit of detection was not low enough, and/or 3) they specifically detected the oxidized form of hIL-33 only. Therefore, caution should be taken when comparing the data generated with these assays.

These results do not show a significant difference between reduced hIL-33 levels from normal and asthmatic patients. One important point to consider is that the samples analyzed were collected from “self-reported” asthmatics, which is likely representative of a heterogeneous population across a range of severities. There is no information on whether these donors were undergoing asthma exacerbation at the time of sample collection, so it cannot be ruled out that reduced hIL-33 would indeed be significantly higher in these instances. It would also be interesting to test sputum samples from moderate-to-severe asthmatics, similarly to Cohen et al, as the human lung tissue contained high levels of hIL-33 (Cohen et al., Nat Commun, 2015, 6:8327).

### Conclusions

In conclusion, commercially available methods to measure reduced hIL-33 concentrations in multiple human matrices did not meet acceptable criteria for sensitivity and specificity. To address this, improved antibodies and a sensitive iPCR assay that can specifically and accurately detect reduced hIL-33 across a wide range of matrices were developed. This novel biomarker assay may have broad implications for ongoing and future clinical trials targeting the hIL-33/hST2 signaling pathway for multiple indications. In a clinical context, it may help identify potential responders based on basal hIL-33 concentrations and determine target engagement for patients receiving anti-hIL-33 antibody treatment.

### Example 3: Reduced IL-33 iPCR Assay

An iPCR assay using DNA-conjugated anti-IL-33 antibody was optimized to quantify reduced hIL-33 in aqueous humor samples from patients with geographic atrophy (GA) secondary to age-related macular degeneration (AMD) (dry AMD).

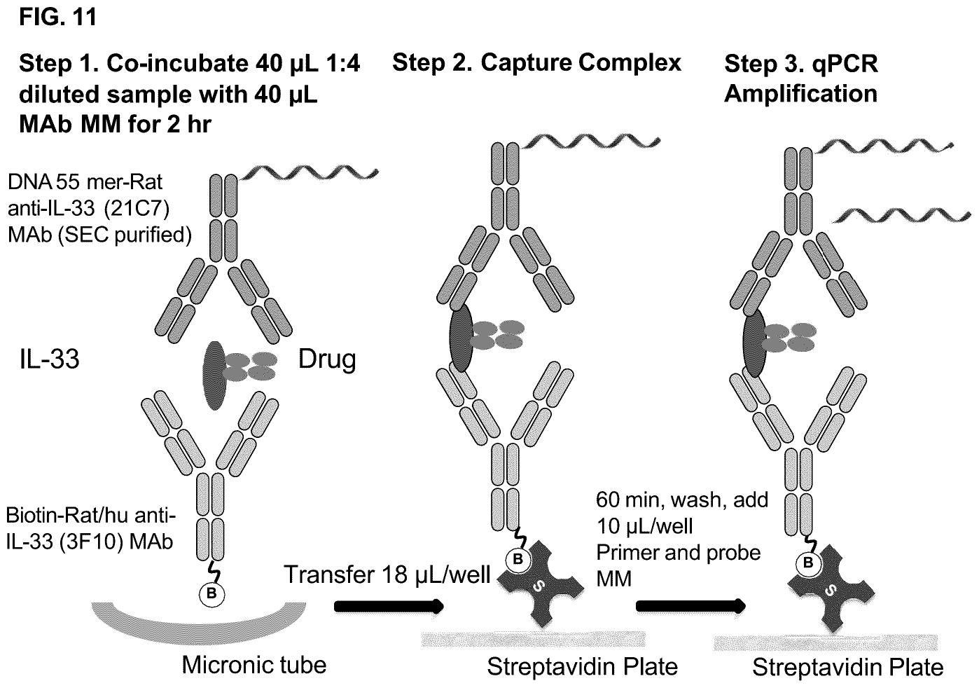

FIG. 11 illustrates the steps of an exemplary IL-33 iPCR assay. Briefly, streptavidin coated 384-well PCR plate (MicroCoat, Cat. 840003) was blocked by adding blocking buffer (Thermo Scientific SUPERBLOCK™ Blocking Buffer Cat. 37515) and incubated for 2-3 hours at room temperature on a shaker. The blocked PCR plate was washed with 25 μL/well of wash buffer (1× phosphate buffered saline (PBS), 0.5% bovine serum albumin (BSA), 15 ppm PROCLIN™). An antibody mixture was prepared by adding 50 ng/ml biotinylated anti-hIL-33 (3F10) rat/human chimeric antibody and 50 ng/mL 55 mer-DNA conjugated rat anti-hIL-33 antibody (21C7). Each of the standard, sample, and QC controls (40 μL) diluted in 1:4 in Sample Diluent (1x PBS, 0.5% BSA, 0.05% polysorbate 20, 15 ppm PROCLIN™ 300, pH 7.4+0.1, 100 μg/mL anti-IL-33 antibody (1E1v8, see WO 2021/183849 and US 2021/0284725 A1, which are incorporated herein by reference in their entirety) and 250 ug/mL heated calf thymus DNA) was mixed with 40 μl of antibody mixture in quadruplicate in 96-well mini tubes and incubated for 2 hours at room temperature on a shaker set at 400 rpm. Addition of the anti-IL-33 antibody 1E1v8 reduces oxidation of IL-33 in the sample. The hIL-33-antibody mixture was added into the well of the blocked PCR plate and incubated at room temperature on a shaker set at 400 rpm. After the incubation the plate was washed and 10 L/well of PCR master mix (1×ABI Universal TAQMAN™ Master Mix II, 1× custom TAQMAN™ gene expression assay mix (Life Technologies, assay ID AIT96PT) containing forward primer (5′-TGAAGGTCCTTGGCGATCA, SEQ ID NO: 39), reverse primer (5′-AGGGACGTAGCCAAATGCAT, SEQ ID NO: 40), and the carboxyfluorescein (FAM)/minor groove binder (MGB)-nonfluorescent quencher (NFQ)-tagged probe (5′-TTCGGCGGTGATCG, SEQ ID NO: 41)) were added. The real-time quantitative PCR (qPCR) amplification was done on a VIIA™ 7 instrument using the following cycle conditions: 95° C. for 10 min, 40 repeats of (95° C. for 15 sec and 60° C. for 1 min). The data were analyzed using SoftMax Pro, version 6.5.1.

### Standard Curve and Control Accuracy, Precision, LLOQ, and ULOQ at Minimum Dilution in Sample Diluent

The standard curve reporting range was determined and the accuracy and precision of the assay within the established reporting range were assessed.

Accuracy is the ability of the analytical method to obtain test results that are close to the true value of the hIL-33. Precision is the degree of agreement among individual test results when the analytical method is repeated for multiple analyses of a sample. Accuracy and precision are also applied to determine the assay's limits of quantification. The lower limit of quantification (LLOQ) is the lowest hIL-33 concentration that can be quantified with acceptable precision and accuracy under the stated experimental conditions. The upper limit of quantification (ULOQ) is the highest hIL-33 concentration that can be quantified for a sample with acceptable precision and accuracy. The reporting range of the assay is the range of concentrations, from the LLOQ to the ULOQ, for which interpolated results have an acceptable level of accuracy and precision.

To determine accuracy, precision, LLOQ, and ULOQ of the assay, samples were first prepared by adding exogenous analyte to Sample Diluent at various concentrations. The samples used to determine the LLOQ were prepared at in-well concentrations of 61, 244, and 977 fg/mL. The samples used to determine the ULOQ were prepared at in-well concentrations of 15,625, 62,500, and 250,000 fg/mL. In addition, low, mid, and high controls spanning the range of the standard curve were prepared at concentrations of 1,000 fg/mL, 8,000 fg/mL, and 800,000 fg/mL, respectively. Endogenous controls were obtained by pooling serum samples with detectable levels of the hIL-33 and were quantitated at 4,000 fg/mL. All samples except LLOQ and ULOQ were then assayed at the minimum dilution in a series of 7 assays over 6 days.

The results of accuracy, precision, LLOQ, and ULOQ at minimum dilution in Sample Diluent are presented in Table 1.

TABLE 11
Standard Curve, Accuracy and Precision

| | Reduced IL-33 | | | Reduced | | | | |
|---|---|---|---|---|---|---|---|---|
| | In-Well | | | IL-33 Mean | | | | Total |
| Standard | Concentration | Mean | CT | Concentration | | | | error |
| Curve | (fg/mL) | CT | Difference | (fg/mL) | % CV | % Recovery | % RE | (%) |
| STD1 | 250000 | 12.7 | 9.3 | 257714 | 22 | 103 | 3 | 25 |
| STD2 | 62500 | 14.8 | 7.3 | 61229 | 10 | 98 | −2 | 12 |
| STD3 | 15625 | 16.9 | 5.2 | 16157 | 13 | 103 | 3 | 16 |
| STD4 | 3906 | 18.8 | 3.3 | 3943 | 12 | 101 | 1 | 13 |
| STD5 | 977 | 20.3 | 1.8 | 1031 | 19 | 105 | 5 | 24 |
| STD6 | 244 | 21.5 | 0.6 | 214 | 15 | 88 | −12 | 27 |
| STD7 | 61 | 22.1 | 0.0 | 69 | 19 | 113 | 13 | 32 |
| Blank | 0 | 22.1 | NA | NA | NA | NA | NA | NA |

CT = cycle threshold; CT Difference = Mean CT of Blank − Mean CT of each standard (STD); CV = coefficient of variation; NA = not applicable; RE = percent relative error; Total error = sum of absolute % RE and % CV.
Note:
Data were summarized from 7 acceptable runs.

The results of the presence of endogenous hIL-33 present in pooled samples are presented in Table 12.

TABLE 12
Endogenous Level of Reduced hIL-33

| | | Reduced hIL-33 |
|---|---|---|
| Sample | N | Concentration (pg/mL) |
| AMD serum | 5 | 1.3-6.1 |
| Asthma serum | 30 | 0.9-10.5 |
| RA serum | 30 | 0.7-15.0 |
| Normal serum pooled | 9 | 3.0-7.2 |
| Normal plasma pooled | 3 | 3.9-4.2 |

AMD = Age-related macular degeneration; RA = Rheumatoid arthritis.

The results of accuracy, precision of high, mid, low, and endogenous controls are presented in Table 13.

TABLE 13
Accuracy and Precision of Controls

| | Reduced IL-33 | | Reduced IL-33 Mean | | | | Total |
|---|---|---|---|---|---|---|---|
| | Concentration | Mean | Concentration | | | | error |
| Control | (fg/mL) | CT | (fg/mL) | % CV | % Recovery | % RE | (%) |
| High | 800000 | 13.2 | 770429 | 12 | 96 | −4 | 16 |
| Mid | 8000 | 19.6 | 8317 | 18 | 104 | 4 | 22 |
| Low | 1000 | 21.4 | 1055 | 19 | 106 | 6 | 25 |
| EC | 4000 | 20.7 | 3547 | 13 | 89 | −11 | 24 |

CT = cycle threshold; CV = coefficient of variation; EC = endogenous control; RE = percent relative error; Total error = sum of absolute % RE and % CV.
Note:
Data were summarized from 7 acceptable runs.

On the basis of the results, accuracy, inter-assay precision, and total error were acceptable for the endogenous, low, mid, and high controls, as well as the selected LLOQ and ULOQ. The LLOQ and ULOQ were selected to be 244 and 250,000 fg/mL, (dilution corrected concentration of 977 and 1,000,000 fg/mL) respectively. The minimum dilution (MRD) for this assay was ¼, which was confirmed during accuracy testing by assaying individual serum samples at the minimum dilution with Sample Diluent.

The minimum quantifiable concentration is equal to the LLOQ multiplied by the minimum dilution required for accurate quantification of the hIL-33 in the sample matrix. Therefore, the minimum quantifiable concentration for this assay is 977 fg/mL.

The maximum quantifiable concentration is equal to the ULOQ multiplied by the maximum sample dilution that can be accurately measured. The maximum quantifiable concentration for this assay was 1,000,000 fg/mL.

### Parallelism (Endogenous Dilution Linearity)

Parallelism is a key experiment that demonstrates if the dose-response of the endogenous hIL-33 in the matrix is sufficiently similar to the dose response of the recombinant standard in the surrogate matrix. It validates the use of the recombinant material as well as the surrogate matrix, and the suitability of the chosen MRD.

Ten individual patient serum samples (1 with AMD, 4 with asthma, and 5 with rheumatoid arthritis) with measurable amounts of hIL-33 were tested at the MRD (¼), followed by one serial ½ dilution in Sample Diluent. The parallelism was acceptable based on 9/10 (90%) samples passing the % CV≤30% criteria and 9/10 (90%) samples passing the % RE for each dilution relative to MRD<30% acceptance criteria. The results are presented in Table 14.

TABLE 14
Parallelism (Endogenous Dilution Linearity) of Reduced hIL-33

| | | | | Reduced | | | | Mean |
|---|---|---|---|---|---|---|---|---|
| | Description | | | hIL-33 | Mean | % Recovery | % Change | % Change |
| Sample | Sample | Lot | Dilution | (fg/mL) | % CV | from MRD | from MRD | from MRD |
| 1 | AMD HSI | BRH1375890 | 4 | 5480 | 9 | 100 | 0 | −12 |
| | | | 8 | 4820 | | 88 | −12 | |
| 2 | Asthma HSI | BRH527265 | 4 | 3670 | 4 | 100 | 0 | 5 |
| | | | 8 | 3860 | | 105 | 5 | |
| 3 | Asthma HSI | BRH527278 | 4 | 4290 | 4 | 100 | 0 | 5 |
| | | | 8 | 4520 | | 105 | 5 | |
| 4 | Asthma HSI | BRH527288 | 4 | 9000 | 2 | 100 | 0 | −3 |
| | | | 8 | 8730 | | 97 | −3 | |
| 5 | Asthma HSI | BRH527291 | 4 | 5120 | 14 | 100 | 0 | 22 |
| | | | 8 | 6260 | | 122 | 22 | |
| 6 | RA HSI | BRH417592 | 4 | 4420 | 6 | 100 | 0 | 9 |
| | | | 8 | 4810 | | 109 | 9 | |
| 7 | RA HSI | BRH417603 | 4 | 4420 | 2 | 100 | 0 | 2 |
| | | | 8 | 4810 | | 102 | 2 | |
| 8 | RA HSI | BRH417605 | 4 | 6670 | 12 | 100 | 0 | 19 |
| | | | 8 | 7940 | | 119 | 19 | |
| 9 | RA HSI | BRH417612 | 4 | 3470 | 34 | 100 | 0 | 63 |
| | | | 8 | 5660 | | 163 | 63 | |
| 10 | RA HSI | BRH417615 | 4 | 9360 | 6 | 100 | 0 | 9 |
| | | | 8 | 10200 | | 109 | 9 | |

AMD = Age - related macular degeneration; CV = coefficient of variation; HSI = human serum individual; MRD = minimum required dilution; RA = rheumatoid arthritis.

### Detectability of Endogenous Level in Human Serum and Plasma

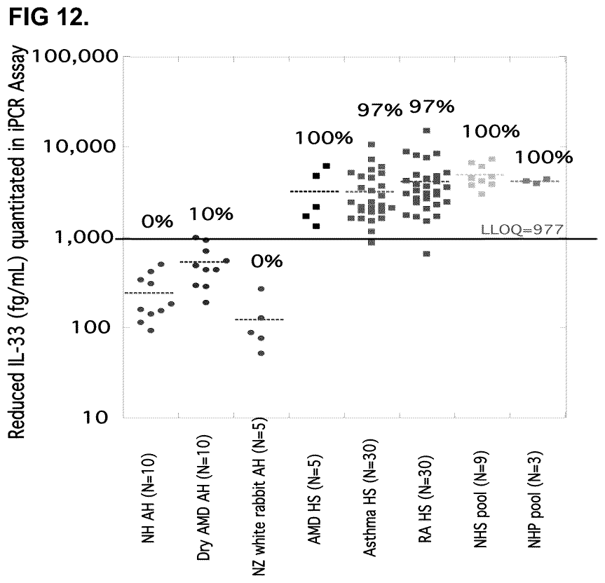

Various aqueous humor, serum, and plasma samples with normal and disease state were diluted to the minimum dilution of ¼ in Sample Diluent and tested for endogenous levels. The detectability of the hIL-33 was acceptable with 97%-100% of the human serum and plasma samples concentration above the LLOQ; however, only 1/20 (5%) human aqueous humor sample was above the LLOQ. The results of detectability are presented in FIG. 12.

### Specificity

Specificity is the ability of the capture antibody to bind selectively to the targeted hIL-33.

Disease state individual samples were incubated with 10 μg/mL of capture antibody for 1 hour at room temperature with shaking. Specificity of the capture antibody was acceptable with 100% inhibition of analyte concentration in all 10 serum samples. The results are presented in Table.

TABLE 15
Specificity

| | | Reduced hIL-33 Conc. (fg/mL) | | |
|---|---|---|---|---|
| | | | Sample with | |
| | | | 10 μg/mL | % |
| Individual | | Sample | Capture | Inhi- |
| Serum Sample | Disease | Alone | MAb | bition |
| 1 | AMD HSI | 4650 | LTR | 100 |
| 2 | AMD HSI | 2680 | LTR | 100 |
| 3 | AMD HSI | 13500 | LTR | 100 |
| 4 | Asthma HSI | 4710 | LTR | 100 |
| 5 | Asthma HSI | 1390 | LTR | 100 |
| 6 | Asthma HSI | 3070 | LTR | 100 |
| 7 | Asthma HSI | 12800 | LTR | 100 |
| 8 | Asthma HSI | 5500 | LTR | 100 |
| 9 | RA HSI | 2720 | LTR | 100 |
| 10 | RA HSI | 2680 | LTR | 100 |
| 11 | RA HSI | 4680 | LTR | 100 |
| 12 | RA HSI | 3410 | LTR | 100 |
| 13 | RA HSI | 11800 | LTR | 100 |

AMD = Age-related macular degeneration; Conc. = concentration; LTR = less than range; HSI = Human serum individual; HSP = Human serum pool; MAb = monoclonal antibody (clone 3F10); RA = rheumatoid arthritis.

### Reproducibility

Reproducibility is the degree of agreement among individual test results when the analytical method is repeated for multiple analyses of a sample measured in an identical fashion over time.

Twenty serum and plasma with detectable levels of hIL-33 were evaluated in 2 runs over a minimum of 2 days. In this experiment, 8/20 (40%) serum and plasma samples showed acceptable reproducibility. Poor reproducibility was probably due to expected oxidation of IL-33 in serum and plasma samples.

The results are presented in Table 15.

TABLE 15
Reproducibility

| | | | | % |
|---|---|---|---|---|
| Individual | | Reduced hIL-33 Conc. (fg/mL) | | Differ- |
| Serum Sample | Disease | Day 1 | Day 2 | ence |
| 1 | HSP | 4200 | 3810 | 9 |
| 2 | HSP | 6630 | 6570 | 1 |
| 3 | HSP | 6050 | 5880 | 3 |
| 4 | HSP | 7290 | 4920 | 33 |
| 5 | AMD HSI | 2170 | 4650 | −114 |
| 6 | AMD HSI | 1330 | 2680 | −102 |
| 7 | AMD HSI | 6090 | 13500 | −122 |
| 8 | Asthma HSI | 7190 | 4710 | 34 |
| 9 | Asthma HSI | LTR | 1390 | 100 |
| 10 | Asthma HSI | 1620 | 3070 | −90 |
| 11 | Asthma HSI | 10500 | 12800 | −22 |
| 12 | Asthma HSI | 5960 | 5500 | 8 |
| 13 | RA HSI | LTR | LTR | 0 |
| 14 | RA HSI | 1750 | 2720 | −55 |
| 15 | RA HSI | 8010 | 2680 | 67 |
| 16 | RA HSI | 8340 | 4680 | 44 |
| 17 | RA HSI | 7520 | 3410 | 55 |
| 18 | RA HSI | 8760 | 9110 | −4 |
| 19 | RA HSI | 3060 | LTR | 100 |
| 20 | RA HSI | 15000 | 11800 | 21 |

AMD = Age-related macular degeneration; Conc. = concentration; LTR = less than range; HSI = Human serum individual; HSP = Human serum pool, RA = rheumatoid arthritis.

### Interference/Cross-Reactivity

Interference demonstrates the ability of an assay to accurately quantitate the analyte in the presence of compounds known to be in systemic circulation at the time of sample collection. Interference is observed if the analyte recovery is <70% or >130% of the nominal analyte concentration in the presence of known concentrations of the potentially interfering molecule.

Cross-reactivity demonstrates the specificity of the assay to only quantitate the analyte and not unrelated compounds that may be in systemic circulation at the time of sample collection.

High, Mid, Low, endogenous, and negative (sample diluent) controls were each assayed with or without 0, 10, 20, 40, and 80 ng/ml of rhST2 (rhST2/IL-33 R Fc Chimera) (R&D Systems Cat. #523-ST-100). No interference on endogenous control was observed with up to 80 ng/ml of rhST2; however, interference on High, Mid, and Low controls was mixed biased over recovery for Mid control at 10 ng/ml rhST2, for High, Mid, and Low control at 20 ng/ml of rhST2, for High and Mid control at 40 ng/ml of rhST2, and for Mid control at 80 ng/ml of rhST2; no interference was observed for High and Low control at 10 ng/ml of rhST2, Low control at 40 ng/ml of rhST2, and High control at 80 ng/mL rhST2. The interference observed in some High, Mid, and Low controls was probably caused by assay variability. No cross-reactivity was observed with up to 80 ng/ml of rhST2. The results are presented in Table 17.

TABLE 17
Interference/Cross-Reactivity

| | | Reduced hIL-33 Conc. (fg/mL) | | | | | | | | | |
|---|---|---|---|---|---|---|---|---|---|---|---|
| | Expected | 0 | | 10 | | 20 | | 40 | | 80 | |
| | Conc. | ng/ml | | ng/ml | | ng/ml | | ng/ml | | ng/ml | |
| Controls | (fg/mL) | rhST2 | % Rec | rhST2 | % Rec | rhST2 | % Rec | rhST2 | % Rec | rhST2 | % Rec |
| High | 800000 | 728000 | 91 | 711000 | 89 | GTR | NA | GTR | NA | 778000 | 97 |
| Mid | 8000 | 6110 | 76 | 10500 | 131 | 15400 | 193 | 29300 | 366 | 13800 | 173 |
| Low | 1000 | 1200 | 120 | 1130 | 113 | 1540 | 154 | 1140 | 114 | LTR | NA |
| Endogenous | 4000 | 2790 | 70 | 5160 | 129 | 2780 | 70 | 5000 | 125 | 4600 | 115 |
| Negative | 0 | LTR | NA | LTR | NA | LTR | NA | LTR | NA | LTR | NA |

rhST2 = rhST2/IL-33 R Fc Chimera; ST2 = suppression of tumorigenicity 2 (receptor for IL-33); Conc. = concentration; GTR = greater than range; LTR = less than range; NA = not applicable; % Rec = % recovery.

### Summary

Table 18 below provides a summary of the reduced IL-33 iPCR assay.

TABLE 18
Summary of the Reduced IL-33 iPCR Assay

| | |
|---|---|
| Analyte | Reduced human IL-33 |
| Matrix | Human aqueous humor, serum, and plasma |
| Reference Standard | Reduced IL-33 his, 1.06 mg/mL in 50 mM Tris, 300 mM NaCl, 10% glycerol, |
| | 1 mM TCEP, pH 8.0, 20 kDa, expressed in E. coli |
| Minimum Dilution | 1/4 |
| LLOQ | 977 fg/mL |
| ULOQ | 1000,000 fg/mL |
| Accuracy (% Bias) | LLOQ: −12%, Low: 6%, Mid: 4%, High: −4%, |
| | ULOQ: 3%, Endogenous: −11% |
| Inter-assay | LLOQ: 15%, Low: 19%, Mid: 18%, High: 12%, |
| precision (% CV) | ULOQ: 22%, Endogenous: 13% |
| Total Error | LLOQ: 27%, Low: 25%, Mid: 22%, High: 16%, |
| of Control | ULOQ: 25%, Endogenous: 24% |
| Parallelism | 9/10 (90%) serum samples had mean % CV ≤ 30 (acceptability is 70%). |
| | 9/10 (90%) serum samples had mean % change from minimum required |
| | dilution (MRD) ≤ ±30 (acceptability is 70%). |
| Detectability | Human aqueous humor (1/20, 5%), serum (72/74, 97%), plasma (3/3, 100%) |
| | (acceptability is > 80%). |
| Specificity | 100% inhibition for 13 of 13 serum samples (acceptability is ≥ 80%). |
| Reproducibility | 8/20 (40%) serum and plasma samples (acceptability is ≥ 80%). |
| Interference/cross- | No interference was observed on endogenous control at up to 80 ng/mL of |
| reactivity | rhST2; no cross-reactivity was observed with up to 80 ng/mL of rhST2 |

### Example 4: Total IL-33 iPCR Assay

An iPCR assay using DNA-conjugated anti-IL-33 antibody was optimized to quantify total hIL-33 in aqueous humor samples from patients with geographic atrophy (GA) secondary to age-related macular degeneration (AMD) (dry AMD). The total IL-33 iPCR assay had the same steps and conditions as the reduced IL-33 iPCR assay described in Example 3, except that prior to mixing the sample with antibody mixture, the sample was treated with 1 mM TCEP for 2 to 4 days at 4° C. Positive control used in the total IL-33 iPCR assay was oxidized IL-33 prepared by diluting IL-33 in 50 mM Tris, 300 mM NaCl, pH 8.0 in Iscove's Modified Dulbecco's Medium (IMDM) and incubating for 1 day at 37° C.

Standard curve and control performance of the total IL-33 iPCR assay are shown in Table 19 below. Minimum dilution was 1:4, LLOQ was 977 fg/mL, and ULOQ was 250,000 fg/mL.

TABLE 19
Standard Curve and Control Performance (N = 7)

| | In-well Conc. | Mean | CT | Results | % | % |
|---|---|---|---|---|---|---|
| Standards | (fg/mL) | CT | diff | (fg/mL) | CV | Recovery |
| STD1 | 250000 | 13.0 | 9.6 | 255000 | 10 | 102 |
| STD2 | 62500 | 15.1 | 7.5 | 59814 | 13 | 96 |
| STD3 | 15625 | 17.1 | 5.5 | 16457 | 11 | 105 |
| STD4 | 3906 | 19.0 | 3.5 | 4137 | 11 | 106 |
| STD5 | 977 | 20.8 | 1.7 | 902 | 17 | 92 |
| STD6 | 244 | 22.0 | 0.6 | 228 | 21 | 93 |
| STD7 | 61 | 22.8 | −0.2 | 71 | 18 | 117 |
| Blank | 0 | 22.5 | | | | |

| | Neat Conc. | Mean | CT | Results | % | % |
|---|---|---|---|---|---|---|
| Controls | (fg/mL) | CT | diff | (fg/mL) | CV | Recovery |
| High | 800000 | 13.2 | 8.9 | 770429 | 12 | 96 |
| Mid | 8000 | 19.6 | 2.5 | 8317 | 18 | 104 |
| Low | 1000 | 21.4 | 0.6 | 1055 | 19 | 106 |
| Endogenous | 4000 | 20.7 | 1.3 | 3547 | 13 | 89 |
| OXC (oxidized | 4000 | 22.9 | −0.1 | LTR | 31 | NA |
| IL-33) | | | | | | |
| Reduced OXC | 4000 | 21.6 | 1.1 | 2196 | 12 | 55 |

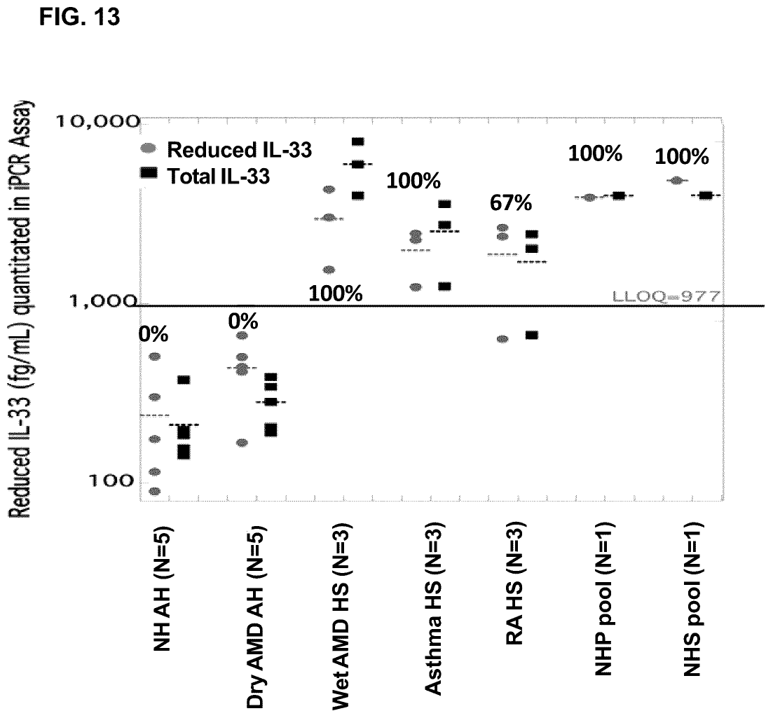

FIG. 13 shows detectability of reduced IL-33 in human humor, serum, and plasma samples with normal and disease state using the reduced or total IL-33 iPCR assays. Reduced IL-33 was only detectable in serums samples of AMD, asthma, and RA patients, as well as normal human serum and normal human plasma pools.

Table 20 below shows reproducibility of the total IL-33 iPCR assay. 8 of 11 (73%) human serum and plasma samples tested showed acceptable reproducibility (i.e., within 30% difference).

TABLE 20
Reproducibility

| | | Day 1 | Day 2 | Day 1 vs 2 |
|---|---|---|---|---|
| Sample # | Sample Description | Total IL-33 Conc. (fg/mL) | | % Difference |
| 1 | Wet AMD human serum (F) | 5950 | 3990 | 33 |
| 2 | Wet AMD human serum (F) | 4000 | 3790 | 5 |
| 3 | Wet AMD human serum (F) | 8000 | 8390 | −5 |
| 4 | Asthma human serum | 2740 | 3160 | −15 |
| 5 | Asthma human serum | 3580 | 5410 | −51 |
| 6 | Asthma human serum | 1250 | 1300 | −4 |
| 7 | Rheumatoid arthritis human serum (F) | LTR | LTR | 0 |
| 8 | Rheumatoid arthritis human serum (F) | 2440 | 3510 | −44 |
| 9 | Rheumatoid arthritis human serum (F) | 2020 | 2610 | −29 |
| 10 | Human plasma pool (sodium citrate) | 3990 | 5040 | −26 |
| 11 | Human serum pool | 4020 | 4050 | −1 |

LLOQ = 977 fg/mL, MRD = 1:4

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### SEQUENCES

| SEQ | | | |
|---|---|---|---|
| ID | Molecule | | |
| NO: | Type | Organism | Sequence |
| 1 | Amino Acid | Synthetic | SQNVH |
| 2 | Amino Acid | Synthetic | RMRFNGDTSYNSTLKS |
| 3 | Amino Acid | Synthetic | QRDNYGSYYFDD |
| 4 | Amino Acid | Synthetic | RASESVLTLLN |
| 5 | Amino Acid | Synthetic | LASHLES |
| 6 | Amino Acid | Synthetic | QQSWIDPWT |
| 7 | Amino Acid | Synthetic | QVQLEESGPGLVQPSQTLSLTCTVSGFSLTSQNVHWVRQPPGK |
| | | | GLEWMGRMRFNGDTSYNSTLKSRLSISRDTSKNQVFLRLNSLQT |
| | | | DDTGTYYCARQRDNYGSYYFDDWGQGIMVTVSS |
| 8 | Amino Acid | Synthetic | DTVLTQSPALAVSPGERVTISCRASESVLTLLNWYQQKPGQHPK |
| | | | LLIYLASHLESGVPARFSGRGSGTDFTLTIDPVEADDTASYYCQQ |
| | | | SWIDPWTFGGGTTLELK |
| 9 | Amino Acid | Synthetic | QVQLEESGPGLVQPSQTLSLTCTVSGFSLTSQNVHWVRQPPGK |
| | | | GLEWMGRMRFNGDTSYNSTLKSRLSISRDTSKNQVFLRLNSLQT |
| | | | DDTGTYYCARQRDNYGSYYFDDWGQGIMVTVSSASTKGPSVFP |
| | | | LAPSSKSTSGGTAALGCLVKDYFPEPVTVSWNSGALTSGVHTFP |
| | | | AVLQSSGLYSLSSVVTVPSSSLGTQTYICNVNHKPSNTKVDKKVE |
| | | | PKSCDKTHTCPPCPAPELLGGPSVFLFPPKPKDTLMISRTPEVTC |
| | | | WVDVSHEDPEVKFNWYVDGVEVHNAKTKPREEQYNSTYRVVS |
| | | | VLTVLHQDWLNGKEYKCKVSNKALPAPIEKTISKAKGQPREPQVY |
| | | | TLPPSREEMTKNQVSLTCLVKGFYPSDIAVEWESNGQPENNYKT |
| | | | TPPVLDSDGSFFLYSKLTVDKSRWQQGNVFSCSVMHEALHNHY |
| | | | TQKSLSLSPGK |
| 10 | Amino Acid | Synthetic | DTVLTQSPALAVSPGERVTISCRASESVLTLLNWYQQKPGQHPK |
| | | | LLIYLASHLESGVPARFSGRGSGTDFTLTIDPVEADDTASYYCQQ |
| | | | SWIDPWTFGGGTTLELKRTVAAPSVFIFPPSDEQLKSGTASVVCL |
| | | | LNNFYPREAKVQWKVDNALQSGNSQESVTEQDSKDSTYSLSSTL |
| | | | TLSKADYEKHKVYACEVTHQGLSSPVTKSFNRGEC |
| 11 | Amino Acid | Synthetic | QVQLEESGPGLVQPSQTLSLTCTVSGFSLT |
| 12 | Amino Acid | Synthetic | WVRQPPGKGLEWMG |
| 13 | Amino Acid | Synthetic | RLSISRDTSKNQVFLRLNSLQTDDTGTYYCAR |
| 14 | Amino Acid | Synthetic | WGQGIMVTVSS |
| 15 | Amino Acid | Synthetic | DTVLTQSPALAVSPGERVTISC |
| 16 | Amino Acid | Synthetic | WYQQKPGQHPKLLIY |
| 17 | Amino Acid | Synthetic | GVPARFSGRGSGTDFTLTIDPVEADDTASYYC |
| 18 | Amino Acid | Synthetic | FGGGTTLELK |
| 19 | Amino Acid | Synthetic | DFWMS |
| 20 | Amino Acid | Synthetic | DIKNDGSHTKYAPSLQN |
| 21 | Amino Acid | Synthetic | VGGTYTAYY |
| 22 | Amino Acid | Synthetic | RTSQGINTYLN |
| 23 | Amino Acid | Synthetic | YTSNLES |
| 24 | Amino Acid | Synthetic | QQYASSPWT |
| 25 | Amino Acid | Synthetic | EVQLVESGGGLVQPGTSLKLSCVASGFTFSDFWMSWVRQTPGK |
| | | | TMEWIADIKNDGSHTKYAPSLQNRFTISRDNAKNTLYLEVTDVRS |
| | | | EDTATYYCTRVGGTYTAYYWGHGVMVTVSS |
| 26 | Amino Acid | Synthetic | DIQMTQTPSSMPASLGERVTLSCRTSQGINTYLNWYQQKPDGTI |
| | | | TPLIHYTSNLESGVPSRFSGSGFGTDYFLTISSLEPEDFAMYFCQ |
| | | | QYASSPWTFGGGTKLELK |
| 27 | Amino Acid | Synthetic | EVQLVESGGGLVQPGTSLKLSCVASGFTFSDFWMSWVRQTPGK |
| | | | TMEWIADIKNDGSHTKYAPSLQNRFTISRDNAKNTLYLEVTDVRS |
| | | | EDTATYYCTRVGGTYTAYYWGHGVMVTVSSAETTAPSVYPLAP |
| | | | GTALKSNSMVTLGCLVKGYFPEPVTVTWNSGALSSGVHTFPAVL |
| | | | QSGLYTLTSSVTVPSSTWSSQAVTCNVAHPASSTKVDKKIVPRE |
| | | | CNPCGCTGSEVSSVFIFPPKTKDVLTITLTPKVTCVVVDISQNDPE |
| | | | VRFSWFIDDVEVHTAQTHAPEKQSNSTLRSVSELPIVHRDWLNG |
| | | | KTFKCKVNSGAFPAPIEKSISKPEGTPRGPQVYTMAPPKEEMTQS |
| | | | QVSITCMVKGFYPPDIYTEWKMNGQPQENYKNTPPTMDTDGSY |
| | | | FLYSKLNVKKETWQQGNTFTCSVLHEGLHNHHTEKSLSHSPGK |
| 28 | Amino Acid | Synthetic | DIQMTQTPSSMPASLGERVTLSCRTSQGINTYLNWYQQKPDGTI |
| | | | TPLIHYTSNLESGVPSRFSGSGFGTDYFLTISSLEPEDFAMYFCQ |
| | | | QYASSPWTFGGGTKLELKKRADAAPTVSIFPPSMEQLTSGGATV |
| | | | VCFVNNFYPRDISVKWKIDGSEQRDGVLDSVTDQDSKDSTYSMS |
| | | | STLSLTKVEYERHNLYTCEVVHKTSSSPVVKSFNRNEC |
| 29 | Amino Acid | Synthetic | EVQLVESGGGLVQPGTSLKLSCVASGFTFS |
| 30 | Amino Acid | Synthetic | WVRQTPGKTMEWI |
| 31 | Amino Acid | Synthetic | RFTISRDNAKNTLYLEVTDVRSEDTATYYCTR |
| 32 | Amino Acid | Synthetic | WGHGVMVTVSS |
| 33 | Amino Acid | Synthetic | DIQMTQTPSSMPASLGERVTLSC |
| 34 | Amino Acid | Synthetic | WYQQKPDGTITPLIH |
| 35 | Amino Acid | Synthetic | GVPSRFSGSGFGTDYFLTISSLEPEDFAMYFC |
| 36 | Amino Acid | Synthetic | FGGGTKLELK |
| 37 | Amino Acid | Synthetic | MHHHHHHGENLYFQG |
| 38 | DNA | Synthetic | TGAAGGTCCTTGGCGATCATTTCGGCGGTGATCGGATGCATTT |
| | | | GGCTACGTCCCT |
| 39 | DNA | Synthetic | TGAAGGTCCTTGGCGATCA |
| 40 | DNA | Synthetic | AGGGACGTAGCCAAATGCAT |
| 41 | DNA | Synthetic | TTCGGCGGTGATCG |

1. An isolated antibody that specifically binds IL-33, or an antigen-binding fragment thereof, wherein the antibody comprises a binding domain comprising a heavy chain variable (VH) domain and a light chain variable (VL) domain, the binding domain comprising the following six complementarity-determining regions (CDRs):
(a) a CDR-H1 comprising the amino acid sequence of SEQ ID NO: 1;
(b) a CDR-H2 comprising the amino acid sequence of SEQ ID NO: 2;
(c) a CDR-H3 comprising the amino acid sequence of SEQ ID NO: 3;
(d) a CDR-L1 comprising the amino acid sequence of SEQ ID NO: 4;
(e) a CDR-L2 comprising the amino acid sequence of SEQ ID NO: 5; and
(f) a CDR-L3 comprising the amino acid sequence of SEQ ID NO: 6.

2-5. (canceled)

6. An isolated antibody that specifically binds IL-33, or an antigen-binding fragment thereof, wherein the antibody comprises a binding domain comprising the following six CDRs:
(a) a CDR-H1 comprising the amino acid sequence of SEQ ID NO: 19;
(b) a CDR-H2 comprising the amino acid sequence of SEQ ID NO: 20;
(c) a CDR-H3 comprising the amino acid sequence of SEQ ID NO: 21;
(d) a CDR-L1 comprising the amino acid sequence of SEQ ID NO: 22;
(e) a CDR-L2 comprising the amino acid sequence of SEQ ID NO: 23; and
(f) a CDR-L3 comprising the amino acid sequence of SEQ ID NO: 24.

7. The antibody of claim 6, comprising (a) a VH domain having at least 95% sequence identity to the amino acid sequence of SEQ ID NO: 25; (b) a VL domain having at least 95% sequence identity to the amino acid sequence of SEQ ID NO: 26; or (c) a VH domain as in (a) and a VL domain as in (b).

8. The antibody of claims 6, wherein the VH domain comprises the amino acid sequence of SEQ ID NO: 25, and the VL domain comprises the amino acid sequence of SEQ ID NO: 26.

9. The antibody of claim 6, wherein the antibody comprises an HC having at least 95% sequence identity to the amino acid sequence of SEQ ID NO: 27; (b) an LC having at least 95% sequence identity to the amino acid sequence of SEQ ID NO: 28; or (c) an HC as in (a) and an LC as in (b).

10. The antibody of claim 6, wherein the HC comprises the amino acid sequence of SEQ ID NO: 27, and the LC comprises the amino acid sequence of SEQ ID NO: 28.

11. The antibody of claim 6, wherein the antibody binds to oxidized or reduced IL-33, and/or the antibody binds to IL-33 bound to soluble interleukin 1 receptor like 1 (sST2) protein and to IL-33 not bound to sST2 with within two-fold K_{D}.

12-14. (canceled)

15. An isolated nucleic acid encoding the antibody of claim 6 or a set of isolated nucleic acids together encoding the antibody.

16. A vector or a set of vectors comprising the isolated nucleic acid or set of isolated nucleic acids of claim 15.

17. A host cell comprising the vector or the set of vectors of claim 16.

18. A method of producing an antibody that specifically binds to IL-33, the method comprising culturing the host cell claim 17 in a culture medium.

19. An immunoconjugate comprising the antibody of claim 6, wherein the antibody is linked to a label.

20. A method of detecting the presence or level of IL-33 in a biological sample comprising:
contacting the biological sample with the immunoconjugate of claim 19 and detecting the presence of the bound immunoconjugate.

21. The method of claim 20, wherein the detecting is by IHC, IF, flow cytometry, ELISA, immunoblotting, or iPCR.

22. The method of claim 20, wherein the detecting is by iPCR, and wherein the method comprises contacting the biological sample with the immunoconjugate in the presence of a second anti-IL-33 antibody that specifically binds an epitope on IL-33 that is different from the epitope to which the anti-IL-33 antibody in the immunoconjugate specifically binds, and detecting the presence of the bound immunoconjugate.

23. The method of claim 20, wherein the biological sample is a blood sample, an ocular sample, a nasal sample, or a cell culture sample.

24. The method of claim 20, wherein biological sample is from a subject having, or at risk of, an IL-33-mediated disorder.

25. (canceled)

26. The method of claim 20, wherein the presence or level of IL-33 is the presence or level of total IL-33, reduced IL-33, or IL-33 bound to sST2.

27-28. (canceled)

29. An assay for identifying a subject having an IL-33 mediated disorder who is a candidate for treatment comprising an IL-33 axis binding antagonist, wherein the assay comprises determining the presence or level of IL-33 in a biological sample obtained from the subject using the immunoconjugate of claim 19.

30. A kit comprising:
(a) the antibody of the immunoconjugate of claim 19; and
(b) a package insert comprising instructions for use of the antibody to detect the presence or level of IL-33 in a biological sample


SEQUENCE LISTING (41 sequences)

SEQ ID NO: 1 - protein, 5 aa, synthetic construct
SQNVH

SEQ ID NO: 2 - protein, 16 aa, synthetic construct
RMRFNGDTSYNSTLKS

SEQ ID NO: 3 - protein, 12 aa, synthetic construct
QRDNYGSYYFDD

SEQ ID NO: 4 - protein, 11 aa, synthetic construct
RASESVLTLLN

SEQ ID NO: 5 - protein, 7 aa, synthetic construct
LASHLES

SEQ ID NO: 6 - protein, 9 aa, synthetic construct
QQSWIDPWT

SEQ ID NO: 7 - protein, 120 aa, synthetic construct
QVQLEESGPGLVQPSQTLSLTCTVSGFSLTSQNVHWVRQPPGKGLEWMGRMRFNGDTSYNSTLKSRLSISRDTSKNQVFLRLNSLQTDDTGTYYCARQRDNYGSYYFDDWGQGIMVTVSS

SEQ ID NO: 8 - protein, 106 aa, synthetic construct
DTVLTQSPALAVSPGERVTISCRASESVLTLLNWYQQKPGQHPKLLIYLASHLESGVPARFSGRGSGTDFTLTIDPVEADDTASYYCQQSWIDPWTFGGGTTLELK

SEQ ID NO: 9 - protein, 450 aa, synthetic construct
QVQLEESGPGLVQPSQTLSLTCTVSGFSLTSQNVHWVRQPPGKGLEWMGRMRFNGDTSYNSTLKSRLSISRDTSKNQVFLRLNSLQTDDTGTYYCARQRDNYGSYYFDDWGQGIMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, synthetic construct
DTVLTQSPALAVSPGERVTISCRASESVLTLLNWYQQKPGQHPKLLIYLASHLESGVPARFSGRGSGTDFTLTIDPVEADDTASYYCQQSWIDPWTFGGGTTLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 30 aa, synthetic construct
QVQLEESGPGLVQPSQTLSLTCTVSGFSLT

SEQ ID NO: 12 - protein, 14 aa, synthetic construct
WVRQPPGKGLEWMG

SEQ ID NO: 13 - protein, 32 aa, synthetic construct
RLSISRDTSKNQVFLRLNSLQTDDTGTYYCAR

SEQ ID NO: 14 - protein, 11 aa, synthetic construct
WGQGIMVTVSS

SEQ ID NO: 15 - protein, 22 aa, synthetic construct
DTVLTQSPALAVSPGERVTISC

SEQ ID NO: 16 - protein, 15 aa, synthetic construct
WYQQKPGQHPKLLIY

SEQ ID NO: 17 - protein, 32 aa, synthetic construct
GVPARFSGRGSGTDFTLTIDPVEADDTASYYC

SEQ ID NO: 18 - protein, 10 aa, synthetic construct
FGGGTTLELK

SEQ ID NO: 19 - protein, 5 aa, synthetic construct
DFWMS

SEQ ID NO: 20 - protein, 17 aa, synthetic construct
DIKNDGSHTKYAPSLQN

SEQ ID NO: 21 - protein, 9 aa, synthetic construct
VGGTYTAYY

SEQ ID NO: 22 - protein, 11 aa, synthetic construct
RTSQGINTYLN

SEQ ID NO: 23 - protein, 7 aa, synthetic construct
YTSNLES

SEQ ID NO: 24 - protein, 9 aa, synthetic construct
QQYASSPWT

SEQ ID NO: 25 - protein, 118 aa, synthetic construct
EVQLVESGGGLVQPGTSLKLSCVASGFTFSDFWMSWVRQTPGKTMEWIADIKNDGSHTKYAPSLQNRFTISRDNAKNTLYLEVTDVRSEDTATYYCTRVGGTYTAYYWGHGVMVTVSS

SEQ ID NO: 26 - protein, 107 aa, synthetic construct
DIQMTQTPSSMPASLGERVTLSCRTSQGINTYLNWYQQKPDGTITPLIHYTSNLESGVPSRFSGSGFGTDYFLTISSLEPEDFAMYFCQQYASSPWTFGGGTKLELK

SEQ ID NO: 27 - protein, 440 aa, synthetic construct
EVQLVESGGGLVQPGTSLKLSCVASGFTFSDFWMSWVRQTPGKTMEWIADIKNDGSHTKYAPSLQNRFTISRDNAKNTLYLEVTDVRSEDTATYYCTRVGGTYTAYYWGHGVMVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 28 - protein, 215 aa, synthetic construct
DIQMTQTPSSMPASLGERVTLSCRTSQGINTYLNWYQQKPDGTITPLIHYTSNLESGVPSRFSGSGFGTDYFLTISSLEPEDFAMYFCQQYASSPWTFGGGTKLELKKRADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 29 - protein, 30 aa, synthetic construct
EVQLVESGGGLVQPGTSLKLSCVASGFTFS

SEQ ID NO: 30 - protein, 13 aa, synthetic construct
WVRQTPGKTMEWI

SEQ ID NO: 31 - protein, 32 aa, synthetic construct
RFTISRDNAKNTLYLEVTDVRSEDTATYYCTR

SEQ ID NO: 32 - protein, 11 aa, synthetic construct
WGHGVMVTVSS

SEQ ID NO: 33 - protein, 23 aa, synthetic construct
DIQMTQTPSSMPASLGERVTLSC

SEQ ID NO: 34 - protein, 15 aa, synthetic construct
WYQQKPDGTITPLIH

SEQ ID NO: 35 - protein, 32 aa, synthetic construct
GVPSRFSGSGFGTDYFLTISSLEPEDFAMYFC

SEQ ID NO: 36 - protein, 10 aa, synthetic construct
FGGGTKLELK

SEQ ID NO: 37 - protein, 15 aa, synthetic construct
MHHHHHHGENLYFQG

SEQ ID NO: 38 - DNA, 55 bp, synthetic construct
  source 1..55: mol_type = other DNA
tgaaggtccttggcgatcatttcggcggtgatcggatgcatttggctacgtccct

SEQ ID NO: 39 - DNA, 19 bp, synthetic construct
  source 1..19: mol_type = other DNA
tgaaggtccttggcgatca

SEQ ID NO: 40 - DNA, 20 bp, synthetic construct
  source 1..20: mol_type = other DNA
agggacgtagccaaatgcat

SEQ ID NO: 41 - DNA, 14 bp, synthetic construct
  source 1..14: mol_type = other DNA
ttcggcggtgatcg